(19) United States
(12) Patent Application Publication
(10) Pub. No.: US 2023/0174633 A1
(43) Pub. Date: Jun. 8, 2023

(54) METHODS AND COMPOSITIONS FOR MODULATING LIPID STORAGE IN ADIPOSE TISSUE

(71) Applicant: MEMORIAL SLOAN KETTERING CANCER CENTER, New York, NY (US)

(72) Inventors: Frederic GEISSMANN, New York, NY (US); Nehemiah COX, New York, NY (US)

(21) Appl. No.: 17/915,990

(22) PCT Filed: Mar. 29, 2021
(86) PCT No.: PCT/US2021/024656

§ 371 Date: Sep. 29, 2022

Related U.S. Application Data
(60) Provisional application No. 63/002,044, filed on Mar. 30, 2020.

(51) Int. Cl. C07K 16/22; C07K 16/28; A61K 38/18; A61K 45/06; A61P 35/00; A61P 3/04

(52) U.S. Cl. CPC C07K 16/22; A61K 38/1858; A61K 38/1866; A61K 45/06; A61P 3/04; A61P 35/00; C07K 16/2866; A61K 2039/505

(57) ABSTRACT

The present technology provides compositions and methods for the treatment of diseases associated with abnormal lipid accumulation. In some embodiments, the present technology also provides methods of treating lipodystrophy, and disorders characterized by abnormal lipid accumulation and/or lipid storage in adipose tissue.

### CROSS-REFERENCE TO RELATED APPLICATIONS

This application claims the benefit of and priority to U.S. Provisional Application No. 63/002,044, filed Mar. 30, 2020, the contents of which are incorporated by reference in their entirety for any and all purposes.

### U.S. GOVERNMENT LICENSE RIGHTS

This invention was made with government support under CA008748, AI130345, HL138090 and CA225036 awarded by the National Institutes of Health. The government has certain rights in the invention.

### TECHNICAL FIELD

The present technology relates generally to the treatment of diseases associated with abnormal lipid accumulation. In particular, the present technology relates to methods of treating lipodystrophy, cachexia, and disorders characterized by abnormal lipid accumulation and/or lipid storage in adipose tissue.

### BACKGROUND

Variation in caloric intake results in cycles of energy storage and expenditure. Chordates have evolved dedicated adipose tissues where specialized fat-storing cells accumulate surplus energy from diet, as triacylglycerols within lipid vacuoles, and release fatty acid via lipolysis when expenditure exceeds intake without the common lipotoxicity associated with fat accumulation in other tissues.

Obesity is associated with a metabolic syndrome characterized by inflammation, insulin resistance, and type 2 diabetes. This syndrome is attributable in part to lipid deposits outside the adipose tissue.

### SUMMARY

In one aspect, the disclosure of the present technology provides a method for treating or preventing lipid accumulation in adipose tissue in a subject in need thereof, comprising administering to the subject an effective amount of a PDGFcc antagonist, an active fragment, or a homolog thereof. In some embodiments, the PDGFcc antagonist is an anti-PDGFcc antibody, or a fragment thereof. In some embodiments, the method comprises selecting for treatment a subject with at least one disease or condition selected from the group consisting of obesity, metabolic syndrome, and hyperlipidemia.

In one aspect, the disclosure of the present technology provides a method for treating or preventing obesity in a subject in need thereof, comprising administering to the subject an effective amount of a PDGFcc antagonist, an active fragment, or a homolog thereof. In some embodiments, the PDGFcc antagonist is an anti-PDGFcc antibody, or a fragment thereof. In some embodiments, the method comprises administering to the subject an effective amount of a combination therapy of: i) a PDGFcc antagonist, an active fragment, or a homolog thereof; and ii) at least one of a CCR2 antagonist or an anti-CCR2 antibody. In some embodiments, the method comprises administering to the subject an effective amount of a combination therapy of: i) an anti-PDGFcc antibody, an active fragment, or a homolog thereof; and ii) at least one of a CCR2 antagonist or an anti-CCR2 antibody. In some embodiments, the obesity is diet-induced or genetic. In some embodiments, the obesity is characterized by adipocyte hypertrophy.

In one aspect, the disclosure of the present technology provides, a method for treating or preventing lipodystrophy in a subject in need thereof, comprising administering to the subject an effective amount of PDGFcc or a PDGFcc agonist, an active fragment, or a homolog thereof. In some embodiments, the method comprises administering to the subject an effective amount of a combination therapy of: i) PDGFcc or a PDGFcc agonist, an active fragment, or a homolog thereof; and ii) at least one of a CCR2 antagonist. In some embodiments, the method comprises administering to the subject an effective of amount of: i) PDGFcc or PDGFcc agonist, an active fragment, or a homolog thereof; and ii) an anti-CCR2 antibody. In some embodiments, the method further comprises administering an effective amount of VEGFb, an active fragment, or a homolog thereof. In some embodiments, the lipodystrophy is hereditary. In some embodiments, the lipodystrophy is associated with HIV infection. In some embodiments, the lipodystrophy is associated with an HIV medication. In some embodiments, the HIV medication is thymidine analogue nucleoside reverse transcriptase inhibitor. In some embodiments, the HIV medication is zidovudine (AZT) or stavudine (d4T). In some embodiments, the HIV medication is an HIV-1 protease inhibitor or nucleoside reverse transcriptase inhibitors (NRTI).

In one aspect, the disclosure of the present technology provides a method for treating or preventing cachexia in a subject in need thereof, comprising administering to the subject an effective amount of PDGFcc or a PDGFcc agonist, an active fragment, or a homolog thereof. In some embodiments, the method comprises administering an effective amount of a combination therapy of: i) PDGFcc or PDGFcc agonist, an active fragment, or a homolog thereof; and ii) at least one of a CCR2 antagonist. In some embodiments, the method comprises administering an effective of amount of: i) PDGFcc or PDGFcc agonist, an active fragment, or a homolog thereof; and ii) an anti-CCR2 antibody. In some embodiments, the method further comprises administering an effective amount of VEGFb, an active fragment, or a homolog thereof. In some embodiments, the method further comprises separately, sequentially, or simultaneously administering an additional treatment to the subject. In some embodiments, the additional treatment comprises administration of a therapeutic agent. In some embodiments, the therapeutic agent is selected from the group consisting of: corticosteroids, such as prednisolone, methylprednisolone, and dexamethasone; progestational agents, such as megestrol acetate and medroxyprogesterone; cannabinoids (dronabinol); serotonin antagonists, such as cyproheptadine; prokinetic agents, such as metoclopramide and cisapride; anabolic steroids, such as nandrolone decanoate and fluoxymesterone; testosterone and its derivatives, such as oxandrolone and enobosarm; ghrelin (anamorelin); inhibitors of phosphoenolpyruvate carboxykinase, such as hydrazine sulfate; methylxanthine analogs, such as pentoxifylline and lisofylline; thalidomide; cytokines and anticytokines, such as anti-IL-6 antibody and IL-12; branched-chain amino acids; eicosapentaenoic acid; inhibitors of prostaglandin synthesis, such as indomethacin and ibuprofen; celecoxib; psychiatric drugs, such as mirtazapine and olanzapine; hormones, such as melatonin; and β₂-adrenocreceptor agonists, such as clenbuterol. In some embodiments, the combination of PDGFcc or a PDGFcc agonist and an additional therapeutic agent has a synergistic effect in the treatment or prevention of cachexia. In some embodiments, the cachexia is associated with cancers of the pancreas, oesophagus, stomach, lung, liver and bowel.

In one aspect, the disclosure of the present technology provides a method to debulk a liposarcoma tumor and facilitate surgical removal of the liposarcoma tumor comprising administering an effective amount of a PDGFcc antagonist, an anti-PDGFcc antibody, or a fragment thereof, to the liposarcoma tumor prior to surgical removal. In some embodiments, the method comprises administering to the subject an effective amount of a combination therapy of: i) a PDGFcc antagonist, an anti-PDGFcc antibody, or an active fragment or a homolog thereof, and ii) at least one of a CCR2 antagonist or an anti-CCR2 antibody.

In one aspect, the disclosure of the present technology provides a method for treating or preventing lipid accumulation in adipose tissue in a subject in need thereof, comprising administering to the subject an effective amount of a PDGFcc antagonist. In some embodiments, the PDGFcc antagonist is an anti-PDGFcc antibody, or a fragment thereof.

In some embodiments, the method comprises selecting for treatment a subject with at least one disease or condition selected from the group consisting of obesity, metabolic syndrome, and hyperlipidemia.

In another aspect, the disclosure of the present technology provides a method for treating or preventing obesity in a subject in need thereof, comprising administering to the subject an effective amount of a PDGFcc antagonist. In some embodiments, the PDGFcc antagonist is an anti-PDGFcc antibody, or a fragment thereof.

In some embodiments, treatment comprises a combination therapy of i) a PDGFcc antagonist, an active fragment, or a homolog thereof and ii) at least one of a CCR2 antagonist or an anti-CCR2 antibody.

In some embodiments, treatment comprises a combination therapy of i) an anti-PDGFcc antibody, an active fragment, or a homolog thereof and ii) at least one of a CCR2 antagonist or an anti-CCR2 antibody

In some embodiments, the obesity is diet-induced or genetic. In some embodiments, the obesity is characterized by adipocyte hypertrophy.

In another aspect, the disclosure of the present technology provides a method for treating lipodystrophy in a subject in need thereof, comprising administering to the subject an effective amount of PDGFcc or a PDGFcc agonist, an active fragment, or a homolog thereof.

In some embodiments, treatment comprises a combination therapy of i) PDGFcc or PDGFcc agonist, an active fragment, or a homolog thereof, and ii) at least one of a CCR2 antagonist or an anti-CCR2 antibody.

In some embodiments, treatment comprises administering an effective of amount of i) PDGFcc or PDGFcc agonist, an active fragment, or a homolog thereof, and ii) an anti-CCR2 antibody. In some embodiments, the method further comprises administration of a VEGFb, an active fragment, or a homolog thereof.

In some embodiments, the lipodystrophy is hereditary. In some embodiments, the lipodystrophy is associated with HIV infection. In some embodiments, the lipodystrophy is associated with an HIV medication. In some embodiments, the HIV medication is thymidine analogue nucleoside reverse transcriptase inhibitor. In some embodiments, the HIV medication is zidovudine (AZT) or stavudine (d4T). In some embodiments, the HIV medication is an HIV-1 protease inhibitor or nucleoside reverse transcriptase inhibitors (NRTI).

In another aspect, the disclosure of the present technology provides a method for treating or preventing cancer-associated cachexia in a subject in need thereof, comprising administering to the subject an effective amount of PDGFcc or a PDGFcc agonist, an active fragment, or a homolog thereof.

In some embodiments, the treatment or prevention comprises a combination therapy of i) PDGFcc or PDGFcc agonist, an active fragment, or a homolog thereof, and ii) at least one of a CCR2 antagonist. In some embodiments, the treatment or prevention comprises administering an effective of amount of i) PDGFcc or PDGFcc agonist, an active fragment, or a homolog thereof, and ii) an anti-CCR2 antibody.

In some embodiments, the method further comprises administrating a VEGFb, an active fragment, or a homolog thereof to the subject.

In some embodiments, the method further comprises separately, sequentially, or simultaneously administering an additional treatment to the subject. In some embodiments, the additional treatment comprises administration of a therapeutic agent. In some embodiments, the therapeutic agent is selected from the group consisting of: corticosteroids, such as prednisolone, methylprednisolone, and dexamethasone; progestational agents, such as megestrol acetate and medroxyprogesterone; cannabinoids (dronabinol); serotonin antagonists, such as cyproheptadine; prokinetic agents, such as metoclopramide and cisapride; anabolic steroids, such as nandrolone decanoate and fluoxymesterone; testosterone and its derivatives, such as oxandrolone and enobosarm; ghrelin (anamorelin); inhibitors of phosphoenolpyruvate carboxykinase, such as hydrazine sulfate; methylxanthine analogs, such as pentoxifylline and lisofylline; thalidomide; cytokines and anticytokines, such as anti-IL-6 antibody and IL-12; branched-chain amino acids; eicosapentaenoic acid; inhibitors of prostaglandin synthesis, such as indomethacin and ibuprofen; celecoxib; psychiatric drugs, such as mirtazapine and olanzapine; hormones, such as melatonin; and β₂-adrenocreceptor agonists, such as clenbuterol. In some embodiments, the combination of PDGFcc or a PDGFcc agonist and an additional therapeutic agent has a synergistic effect in the treatment or prevention of cachexia. In some embodiments, the cachexia is associated with cancer. In some embodiments the cancer is selected from cancers of the pancreas, oesophagus, stomach, lung, liver and bowel.

In another aspect, the disclosure of the present technology provides a method to debulk a liposarcoma tumor and facilitate surgical removal of the liposarcoma tumor comprising administering an effective amount of a PDGFcc antagonist, an anti-PDGFcc antibody, or a fragment thereof, to the liposarcoma tumor prior to surgical removal. In some embodiments, treatment comprises a combination therapy of i) a PDGFcc antagonist, an anti-PDGFcc antibody, or an active fragment or a homolog thereof, and ii) at least one of a CCR2 antagonist or an anti-CCR2 antibody.

### BRIEF DESCRIPTION OF THE DRAWINGS

FIGS. 1A-1B show the tSNE plot and tSNE color coded flow plot representation of flow cytometry analysis of F4/80+ cells among the stromal vascular fraction from the iWAT of P26 Csf1r^{f/f} mice (a, controls, n=15), Csf1r^{Cre}; Csf1r^{f/f} (b, n=4, from 2 litters), Flt3^{Cre}; Csflr^{f/f} (b, n=5, from 3 litters), CCR2^{-/-} (b, n=4, from 2 litters) mice, and Tnfrsf11a^{Cre};Csf1r^{f/f} (b, n=9, from 3 litters). Bottom panel represent May-Grunwald Giemsa staining of FACS-sorted macrophages populations from C57B1/6J mice.

FIG. 1C shows the flow cytometry quantification of adipose tissue F4/80+ macrophages gated as in FIG. 7A in P26 iWAT of Csflr^{Cre}; Csf1r^{f/f} (n=4, from 2 litters), Flt3^{Cre}; Csflr^{f/f} (n=5, from 3 litters), CCR2^{-/-} (n=4, from 2 litters), and their littermate controls (n=4-5, from 2-3 litters), Tnfrsf11a^{Cre}; Csf1r^{ff}(n=6 from 3 litters) mice and littermate controls (n=9 from 3 litters). p values for total F4/80+ cell count was obtained by t-test.

FIG. 1D shows the gross morphology of iWAT and weights of iWAT, eWAT, and iBAT from P26 Csflr^{Cre}; Csflr^{f/f} and control littermates (n=4-5 from 2 litters for Csflr^{Cre}; Csf1r^{f/f} mice; n=6-8, from 3 litters for Csf1r^{f/f} mice).

FIGS. 1E-1F show the gross morphology of iWAT and weights of iWAT, eWAT, and iBAT from P26 Flt3^{Cre}; Csf1r^{f/f} CCR2^{-/-}, and control littermates (n=7-9, from 5 litters for Flt3^{Cre}; Csflr^{f/f} mice, n=3-7, from 3-4 litters for Csflr^{f/f} mice; n=6, from 2 litters for CCR2^{-/-} mice, n=3, from 2 litters for CCR2^{+/-} mice).

FIGS. 1G-1H show the gross morphology of P26 iWAT from Tnfrsf11a^{Cre}; Csflr^{f/f} (n=9, from 4 litters), Tnfrsf11a^{Cre}; PU.1^{f/f} (n=5, from 3 litters), and control littermates (n=5-10, from 3-5 litters). iWAT, eWAT, and iBAT weights from P26 Tnfrsf11a^{Cre}; Csflr^{f/f} mice and littermate controls.

FIG. 1I shows the liver triglycerides (TG), as µg per mg of tissue at P26 (n=4, from 2 litters).

FIG. 1J shows the representative flow cytometry plot of iWAT from 1 month old Csf1r^{MeriCreMer};Rosa^{LSL-YFP} mice pulse labeled with 4-hydroxy tamoxifen at E8.5. (Flow plot representative of n=4, from 2 litters). Each dot represents one mouse, p values obtained by one-way ANOVA with Sidak’s correction unless otherwise indicated.

FIG. 1K depicts a parsimony diagram for the control of adiposity by macrophages. Resident macrophages (purple) produce PDGFcc in response to diet (large purple arrow), which regulates lipid storage in adipocytes. PDGFcc blockade results in reduced storage by adipocytes and increased thermogenesis in brown adipose tissue (black arrow). In contrast, bone-marrow-derived macrophages (red) recruited to hypertrophic adipocytes (large red arrow) produce TNF and IL1 1b that mediate hepatosteatosis and insulin resistance (red arrows)..

FIG. 2A shows the weight of eWAT at 7, 14, and 26 days after birth (n=4-13, from 3-7 litters). p values obtained by two-way ANOVA.

FIG. 2B shows the perilipin staining on paraffin-embedded tissue sections from iWAT of 7 and 26 days old Tnfrsf11a^{Cre}; Csf1r^{f/f} (n=9, from 5 litters) and littermate Csflr^{f/f} mice (n=10, from 5 litters).

FIG. 2C shows the surface area distribution of iWAT adipocytes from 7 and 26 days old Tnfrsf11a^{Cre}; Csf1r^{f/f} and littermate Csf1r^{f/f} mice as determined from perilipin stained paraffin-embedded tissue sections (n=3, 3 litters, 100-200 adipocytes per replicate). Each point represents average size calculated from paraffin sections.

FIG. 2D shows the qPCR analysis of the indicated genes in total iWAT from P7 or P26 Tnfrsf11a^{Cre}; Csf1r^{f/f} and littermate control mice (n=6-8, from 4 litters).

FIG. 2E shows the fluorescent whole mount images of P26 iWAT from Tnfrsf11a^{Cre}; Csflr^{f/f} and Csf1r^{f/f}littermate control mice (n=4, from 4 litters) stained for DAPI, CD31, PDGFRα, Perilipin, Bodipy, and F4/80.

FIG. 2F shows the volume distribution of iWAT adipocytes from P26 Tnfrsf11a^{Cre}; Csflr^{f/f} and littermate Csf1r^{f/f} mice as determined from whole mount imaging as represented in FIG. 2E. Each point represents average size calculated from fluorescent whole mount images (n=3, from 3 litters, 100-200 adipocytes per replicate).

FIG. 2G shows the quantification of PDGFRα⁺ cells from iWAT paraffin embedded tissue sections of P26 mice. Each dot represents an independent value obtained from an average count of 3 fields from a single paraffin embedded iWAT section (n=3 from 3 litters for Csflr^{f/f} and Tnfrsf11a^{Cre}; Csf1r^{f/+}, n=6 from 3 litters for Tnfrsf11a^{Cre}; Csf1r^{f/f}.

FIG. 2H shows the qPCR analysis of the indicated growth factors in total iWAT from P7 Tnfrsf11a^{Cre}; Csflr^{f/f} and littermate control mice (n=4-6, from 2-3 litters).

FIG. 2I shows the qPCR analysis of the indicated growth factors in FACS-sorted iWAT macrophages from 4 week old C57Bl/6J mice. 2^{-ΔCt} calculated relative to Gapdh and ActinB. Each dot represents one mouse, p values obtained by one-way ANOVA unless otherwise stated (n=10-30).

FIG. 2J shows the fluorescent whole mount staining of P26 iWAT from Tnfrsf11a^{Cre}; Csflr^{f/f} and Csflr^{f/f}littermate control mice (n=5, from 4 litters) with F4/80 and PDGFcc antibodies and Bodipy, and quantification of expression of PDGFcc⁺by F4/80⁺ cells.

FIG. 3A shows the fluorescent images of L3 larvae depicting the expression pattern of hemocyte-specific drivers Hemolectin (Hml) and Serpent (Srp) as indicated by GFP and mCherry, respectively (n=3). Whole mount image of Srp^{Hemo}-mCherry hemocytes in contact with GFP⁺ fat body cells in Srp^{Hemo}-mCherry; c564-gal4>uas-GFP L3 larvae (n=3).

FIG. 3B shows the triglyceride level (TG) from L3 larvae of Srp^{Hemo}-gal4>uas-reaper, Hml-gal4>uas-reaper, and control larvae (n=9-11, from 4 crosses, 10 larvae/replicate). TG values were normalized to the total protein level in each analyzed sample, p values obtained by one-way ANOVA with Sidak’s correction.

FIG. 3C shows the size distribution of Bodipy⁺ fat body cells in wandering L3 Srp^{Hemo}-gal4>uas-reaper (n=6, from 3 crosses), Hml-gal4>uas-reaper (n=9, from 4 crosses), and control larvae (n=6, from 3 crosses). 100 fat body cells were examined for each replicate. p values obtained by t-test comparing mean cell size. Each dot represents mean fat body cell size.

FIG. 3D shows the quantitative analysis of buoyancy in wandering L3 larvae from Srp^{Hemo}-gal4>uas-reaper and control lines (n=6, from 3 crosses). Each replicate includes 20 larvae.

FIG. 3E shows the normalized TG level in wandering L3 larvae from flies with hemocyte-specific RNAi of the indicated growth factor (n=8-12, from 4 crosses, 10 larvae/replicate). p values obtained by one-way ANOVA with Sidak’s correction.

FIG. 3F shows the quantification of Bodipy⁺ fat body cell size in L3 larvae (n=3-6, from 3-6 crosses, 25-50 cells/replicate). Each dot represent a single fat body cells. p values obtained by one-way ANOVA with Dunnett’s correction.

FIG. 3G shows the size distribution of Bodipy⁺ fat body cells in L3 Srp^{Hemo}-gal4>uas-pvf3-IR (n=3, from 3 crosses) and control larvae (n=10, from 3 crosses). Each dot represents mean fat body size. 100 fat body cells were examined for each replicate, p values obtained by t-test comparing mean cell size.

FIG. 3H shows the normalized TG level in wandering L3 larvae from flies with fat body-specific RNAi of the Pvf receptor, Pvr (n=8-1 1, from 4 crosses, 10 larvae/replicate). p values obtained by t-test.

FIG. 3I shows the quantitative analysis of wandering L3 larvae buoyancy from Pvf3^{EY09531} and control y¹ w1¹¹¹⁸ L3 larvae. Buoyancy assay from Pvf3^{EY09531} mutant and control L3 larvae (n=6, from 3 crosses). Each replicate includes 20 larvae.

FIG. 3J shows the size distribution of Bodipy⁺ fat body cells, p values obtained by t-test comparing mean fat body cell size (n=2-5, from 3 crosses). Each dot represents mean fat body cell size. 100 fat body cells were examined for each replicate.

FIG. 3K shows the TG level in L3 larvae (n=7-12, from 4 crosses, 10 larvae/replicate). p values obtained by t-test.

FIG. 4A shows a schematic depicting the isolation and ex vivo culture of eWAT anlage from 4 days old Tnfrsf11a^{Cre}; Csflr^{f/f} or littermate Csf1r^{f/f} mice. GF: growth factor.

FIG. 4B shows the fluorescent whole mount images of eWAT from 4-day-old Csflr^{f/f} mice (n=3).

FIGS. 4C-4D show the representative whole mount images of eWAT explants cultured for 10 days ex vivo from Tnfrsf11a^{Cre}_{;} Csf1r^{f/f} (n=10) or Csf1r^{f/f} (n=15 ) littermate control mice.

FIG. 4E shows the total area and size distribution of Bodipy⁺ area from Csf1r^{f/f} or Tnfrsf11a^{Cre}; Csflr^{f/f} eWAT explants with the indicated treatment (n=5-15). p values obtained by one-way ANOVA.

FIG. 4F shows the fluorescent whole mount images of eWAT explants from Csf1r^{f/f} mice supplemented with Goat IgG or anti-PDGFcc neutralizing antibodies (n=5).

FIG. 4G shows the fluorescent whole mount images of eWAT explants from Tnfrsf11a^{Cre}; Csf1r^{f/f} mice supplemented with vehicle control (PBS), PDGFcc, or IGF (n=3-5).

FIG. 4H shows the qPCR analysis of the indicated genes in total eWAT explants (n=8-9). 2^{-ΔCt} calculated relative to Gapdh, n.s. obtained by t-test.

FIG. 5A shows the mouse body weight over time fed the indicated diet (n=5). Mean ± SD, p values at 12 weeks of age obtained by t-test.

FIG. 5B shows the weight of adipose tissues from 12 weeks old C57B1/6J mice under the indicated diet and treatment (n=9 for 45%, 45% treated with anti-PDGFcc antibody, and 10% diet in control mice, n=4 for 10% fed mice treated with anti-PDGFcc antibody, from 2 independent experiment).

FIG. 5C shows the size distribution of iWAT adipocytes from anti-PDGFcc antibody treated mice and controls with the indicated diet (n=4). Each dot represents mean adipocyte size. 100-200 adipocytes were examined for each replicate.

FIG. 5D shows the fluorescent whole mount images of iWAT adipocytes from anti-PDGFcc antibody treated mice and controls (n=4).

FIG. 5E shows that C57B1/6J mice were injected with anti-PDGFcc antibodies or goat IgG and their cumulative food intake was measured gravimetrically for 5 days over the first week of treatment (n=8).

FIG. 5F shows that feces were collected from C57B1/6J mice injected with anti-PDGFcc antibodies or goat IgG on day 6 of treatment and then subjected to bomb calorimetry to measure fecal caloric density (n=8).

FIG. 5G shows that C57B1/6J mice injected with anti-PDGFcc antibodies or goat IgG were single housed in a Promethion Metabolic Screening System to measure O₂ and CO₂ levels over 5 days during the first week of treatment (n=8). Analysis of covariance corrected cumulative energy expenditure of C57B1/6J mice fed a 45% fat diet and injected with anti-PDGFcc antibodies or goat IgG over a period of 5 days during the first week of treatment (n=8).

FIG. 5H shows the representative infrared images of C57B1/6J mice injected with anti-PDGFcc antibodies or goat IgG on day 6 of treatment. Top 10% warmest pixels were used for the quantification of iWAT and iBAT surface area temperatures. Dorsal surface area temperature was calculated using top 20% warmest pixels (n=8).

FIG. 5I shows the qPCR analysis of Ucp1 and Dio2 transcripts in iBAT of 12 week old C57B1/6J mice injected with anti-PDGFcc antibodies or goat IgG for 6 weeks (n=8). 2^{-ΔCt} calculated relative to Gapdh, p values obtained by t-test.

FIG. 5J shows the representative H&E staining of livers from mice on the indicated diet and treatment (n=4 from 2 independent experiments).

FIG. 5K shows the liver Triglycerides per mg of tissue in mice on the indicated diet and treatment (n=8 for 45%, 45% treated with anti-PDGFcc antibody, and 10% diet in control mice, n=4 for 10% fed mice treated with anti-PDGFcc antibody).

FIG. 5L shows the qPCR analysis of Tnf and Il1b transcripts in the liver of mice fed 10% or 45% fat diet with the indicated treatment, normalized to Gapdh (2^{-ΔCt}). Each dot represents one mouse (n=8 for 45%, 45% treated with anti-PDGFcc antibody, and 10% diet in control mice, n=4 for 10% fed mice treated with anti-PDGFcc antibody). p values obtained by one-way ANOVA with Sidak’s correction unless otherwise stated.

FIG. 6A shows the fluorescent whole mount image of Tim4+ macrophages surrounding adipocytes in the iWAT of lean 14 week old C57B1/6J mice.

FIG. 6B shows the representative flow plots depicting expression of Tim4, CD11c, MHCII, and forward and side size scatter (SSC and FSC) by the indicated color-coded clusters in eWAT of 14 week-old mice as determined by flow cytometry analysis (n=5).

FIG. 6C shows the flow cytometry quantification of eWAT adipose tissue macrophages from C57Bl/6J, Ccr2^{-/-} mice, and Ccr2^{+/-} littermate controls (n=4 fed a 10% lipid diet, n=5 fed a 45% lipid diet).

FIG. 6D shows the flow cytometry analysis of blood monocytes and adipose tissue macrophages from iWAT of parabiotic pairs between CD45.1 and CD45.2 females fed 10% or 45% lipid diet. 2 pairs were analyzed per group, each dot represents one mouse.

FIG. 6E shows the expression of indicated genes, relative to Gapdh and Actinb (2⁻^{ΔCt}), as determined by qPCR in FACS-sorted iWAT macrophage subsets and blood monocytes in 14-week-old C57B1/6J fed a 10% or 45% lipid diet for 8 weeks (n=7 from 2 independent experiments).

FIG. 6F shows the qPCR analysis of Pdgfc transcripts in total iWAT, normalized to Gapdh (2^{-ΔCt}). Ccr2^{+/-} (n=6, from 2 litters), and littermate controls (n=7, from 2 litters) were fed 10% or 45% diets for 8 weeks. C57B1/6J mice were treated with PLX5622 (n=8), aPDGFcc neutralizing antibodies (n=8), a-CSF1R blocking antibodies (n=5), or the appropriate controls (n=10 for 10% and n=15 for 45%) for 6 weeks on the indicated diet.

FIG. 6G shows the weight of adipose tissues from mice with the indicated genotypes and treatments fed 10% or 45% fat diet. Ccr2^{+/-} (n=9, from 3 litters), and littermate controls (n=8, from 3 litters) were fed 10% or 45% diets for 8 weeks. C57B1/6J mice were treated with PLX5622 (n=11 for 45% and n=8 for 10%, from 2 experiments), a-CSF1R blocking antibodies (n=5), or the appropriate controls (n=17 for 10% and n=21 for 45%, from 4 experiments) for 6 weeks on the indicated diet.

FIG. 6H shows the fluorescent whole mount images of iWAT adipocytes from mice on the indicated diet and treatment as in FIG. 6G.

FIG. 6I shows the size distribution of iWAT adipocytes from Ccr2^{+/-} and littermate controls fed 10% or 45% diet. Each dot represents mean adipocyte size, 100-200 adipocytes per replicate, n=3. p values obtained by t-test comparing mean adipocyte size from mice fed a 45% lipid diet to 10% lipid diet.

FIG. 6J shows the size distribution of iWAT adipocytes from the indicated treatments, p values obtained by t-test. Each dot represents mean adipocyte size, 100-200 adipocytes per replicate, n=3. p values obtained by t-test comparing mean adipocyte size from mice fed a 45% lipid diet to 10% lipid diet and 45% lipid diet to 45% + PLX5622 or 45% + a-CSF1R antibody.

FIG. 6K shows the cumulative food intake was measured gravimetrically in 7 and 12-week-old C57B1/6J mice fed the indicated diet (n=8).

FIG. 6L shows that feces were collected from 12-week-old C57B1/6J mice fed the indicated diet and then subjected to bomb calorimetry to measure fecal caloric density (n=8).

FIG. 6M shows the weight of adipose tissues from 12-week-old Lepr ^{-/-} mice fed the indicated diet for 8 weeks (n=4).

FIG. 6N shows the fluorescent whole mount images of iWAT adipocytes from PLX5622-treated and control Lepr ^{-/-} mice (n=4). Size distribution of iWAT adipocytes from PLX5622-treated and control Lepr ^{-/-} mice. Each dot represents mean adipocyte size, 100-200 adipocytes per replicate, n=4. p values obtained by t-test.

FIG. 6O shows the cumulative food intake was measured gravimetrically in 7-week-old Lepr ^{-/-} mice fed the indicated diet (n=4).

FIG. 6P shows that feces were collected from 7-week-old Lepr ^{-/-} mice fed the indicated diet and then subjected to bomb calorimetry to measure fecal caloric density (n=4). Each dot represents one mouse, p values are obtained by one-way ANOVA with Sidak’s correction unless otherwise stated.

FIG. 7A shows the gating strategy used for flow analysis and flow sorting of different macrophage populations in the adipose tissues studied, the eWAT is represented here (representative of n=15). The same gating was used in iWAT and mWAT. Lin is CD3, CD19, NKp46, and SiglecF.

FIG. 7B shows the photographs of whole mice, eWAT, and iWAT from Csf1r^{-/-} mice (n=3, from 3 litters) and control littermates (n=3, from 3 litters) at P21.

FIG. 7C shows the photographs of whole mice and eWAT from Csf1r^{Cre};Csf1r^{f/f} mice (n=4, from 2 litters) and control littermates (n=4, from 2 litters) at P26.

FIG. 7D shows the absolute weight (top graph) and relative weight normalized to body weight (bottom graph) of brain, liver, lungs, kidney, WAT (eWAT, iWAT and mWAT) and iBAT from Csf1r^{Cre};Csf1r^{f/f} mice (n=4, from 3 litters) and control littermates (n=6-8, from 4 litters) at P26.

FIG. 7E shows the H&E staining of livers from Csf1r^{Cre};Csf1r^{f/f} mice (n=4, from 2 litters) and control littermates (n=4, from 2 litters) at P26.

FIGS. 7F-7G show the representative images of eWAT from Flt3^{Cre};Csf1r^{f/f} (n=5, from 3 litters) , Ccr2 ^{-/-} (n=4, from 2 litters), and their littermate controls at P26. Each dot represents one mouse, p values obtained by one-way ANOVA with Sidak’s correction unless otherwise indicated.

FIG. 8A shows the photographs of whole mice and eWAT, from Tnfrsf11a^{Cre} ; Csf1r^{f/f} mice (n=9, from 5 litters) and control littermates (n=13, from 5 litters) at P26.

FIG. 8B shows the perilipin staining of paraffin embedded eWAT and skin sections from Tnfrsf11a^{Cre}; Csflr^{f/f} (n=9, from 4 litters) and littermate control (n=10, from 4 litters) mice at P26.

FIG. 8C shows the absolute weight (top graph) and relative weight normalized to body weight (bottom graph) of liver, lungs, kidney, WAT (eWAT, iWAT and mWAT) and iBAT from Tnfrsf11a^{Cre}; Csflr^{f/f} mice (n=4, from 3 litters) and control littermates (n=4, from 3 litters) at P26.

FIG. 8D shows the photographs of whole mice and eWAT, from Tnfrsf11a^{Cre} ; Pu. 1 ^{f/f}mice (n=5, from 3 litters) and control littermates (n=5, from 3 litters) at P26.

FIG. 8E shows the absolute weight (top graph) and relative weight normalized to body weight (bottom graph) of liver, iWAT and iBAT from Tnfrsf11a^{Cre}; Pu.1^{f/f} mice (n=5, from 3 litters) and control littermates (n=5, from 3 litters) at P26.

FIG. 8F shows the photography and H&E staining of liver from Tnfrsf11a^{Cre}; Csflr^{f/f} mice and littermate controls, at P26 (n=8-10, from 4 litters).

FIG. 8G shows the perilipin and UCP1 staining of paraffin embedded iBAT, and qPCR analysis of Ucp1 and Dio2 transcripts normalized to Gapdh from iBAT from Tnfrsf11a^{Cre}; Csf1r^{f/f}(n=9, from 4 litters) and littermate control mice (n=8, from 4 litters) at P26.

FIG. 8H shows the weight of iWAT and mWAT inTnfrsf11a^{Cre};Csf1r^{f/f} and littermates at P7, P14 and P26 (n=4-13, from 3-8 litters). Scale bars represent indicated indicated value. Each dot represents one mouse. p values obtained by one-way ANOVA with Sidak’s correction unless otherwise indicated.

FIG. 9A shows the whole mount images of 1 month old iWAT from Csflr^{Cre}; mTmG and Tnfrsf11a^{Cre}; mTmG mice (n=3, from 2 litters). Arrow in FIG. 9A points to a F4/80⁺ Tim4⁻ macrophage. Scale bars represent 20 mm.

FIG. 9B shows the quantification of Bodipy⁺ area from the indicated eWAT explants (n=4-7). p values obtained by one-way ANOVA with Sidak’s correction.

FIG. 10A shows the confocal image of control (left, HmlΔ-gal4>uas-GFP) (n=10) and hemocyte-less L3 larvae (right, HmlΔ-gal4>uas-GFP,uas-reaper) (n=6).

FIG. 10B shows the qPCR analysis of the indicated growth factors in FACS-sorted hemocytes from HmlΔ-gal4>uas-GFP larvae. 2^{-ΔCt} is calculated relative to Gapdh, (n=4-9, 20 L3 larvae per replicate).

FIG. 10C shows the qPCR analysis of the indicated growth factors in whole larvae in control Srp^{Hemo}-gal4 L3 larvae, hemocyte specific RNAi of pvf1 (left) and of pvf3 (right), n=3-6 replicates, with 10 larvae per replicate.

FIG. 10D shows the size distribution of Bodipy+ fat body cells in L3 larvae (n=3, from 3 crosses, 100-150 cells per replicate). Each dot represents mean fat body cell size. p values obtained by t-test comparing mean cell size.

FIG. 10E shows the developmental analysis of the larvae with the indicated genotype following egg deposition (n=3, from 30 larvae per replicate). The dashed lines indicate 95% confidence interval, p values obtained by t-test.

FIG. 10F shows the size of L3 larvae from the indicated genotypes (n=10-20, from 5 larvae per replicate). Each dot represent mean values.

FIG. 11A shows the flow cytometry quantification of iWAT adipose tissue macrophages from C57B1/6J mice fed the indicated diet and injected with anti-PDGFcc antibodies or goat IgG (n=5 for 10%, n=9 for 45%, and n=9 for 45% + anti-PDGFcc antibody). Each dot represents one mouse, p values obtained by one-way ANOVA with Sidak’s correction.

FIG. 11B shows the flow cytometry quantification of liver Kuppfer cells from C57B1/6J mice fed the indicated diet and injected with anti-PDGFcc antibodies or goat IgG (n=4).

FIG. 11C shows the cumulative ambulatory activity was acquired using a laser matrix over a period of 5 days during the first week of anti-PDGFcc blocking antibodies or goat IgG treatment (n=8). Each dot represents the mean ± SD.

FIG. 11D shows the glucose Insulin and tolerance test on 12-week-old mice with the indicated diet and treatment (n=4). Each dot represents the mean ± SD. Each dot represents one mouse unless stated otherwise. Blood glucose concentration from the glucose tolerance test was normalized to the initial blood glucose level (time =0) and then the area under the curve was measured (n=4). Each dot represents one area under the curve from one mouse.

FIG. 11E shows the top 10% warmest pixels were used for the quantification of iWAT and iBAT surface area temperatures. Dorsal surface area temperature was calculated using top 20% warmest pixels. n.s. obtained by t-test (n=8).

FIG. 11F shows the qPCR analysis of ucp1 and Dio2 transcripts in iBAT of 12 week old C57B1/6J mice injected with anti-PDGFcc antibodies or goat IgG for 6 weeks (n=4). 2^{-ΔCt} calculated relative to Gapdh, p values obtained by t-test.

FIG. 12A shows the tSNE analysis of flow cytometry data for stromal vascular fraction from the iWAT of 14 weeks old C57Bl/6J mice fed the indicated diet. The t-SNE algorithm was calculated on the singlets using the parameters: CD45, CD3, CD19, NKp46, SiglecF, Ly6G, F4/80, CD11b, Tim4, CD11c and MHCII, as well as FSC-A and SSC-A (representative of n=15).

FIG. 12B shows the flow cytometry characterization of Tim4⁺ and Tim4⁻ macrophages color-coded in the tSNE plot in lean 8 weeks old mice. For F4/80, CD11b, CD11c, Tim4 and MHCII FMO were used. Anti-CD64, MerTK and Ly6C were compared to isotypes controls. The FMO and isotype controls were prepared from a mix of cells from the different adipose tissues and used to compare the expression of markers in all tissues. Expression of Cx3cr1 is measured in the Cx₃cr1^{GFP/+} mice at 2 months of age and compared to a littermate controls (n=3).

FIG. 13A shows the gating strategy used for flow analysis and flow sorting of different macrophage populations in the adipose tissues studied, the eWAT is represented here (representative of n=15). The same gating was used in iWAT, mWAT and iBAT as well. Lin-1 is CD3 CD19 NKp46, Lin-2 is SiglecF Ly6G.

FIG. 13B shows the gating strategy for analysis of the blood Ly6C^{hi} monocytes.

FIG. 14A shows the quantification of Tim4⁺ and Tim4⁻ macrophage populations in iWAT, and mWAT by flow cytometry in Ccr2^{-/-} mice (n=4, from 2 litters) and littermate controls (n=5, from 2 litters) fed 10% or 45% lipid diet. Flow cytometry quantification of the blood Ly6C^{hi} monocytes (n=4-5, from 2 litters).

FIG. 14B shows the flow cytometry analysis of liver Kupffer cells and adipose tissue macrophages from mWAT of parabiotic pairs between CD45.1 and CD45.2 females fed 10% or 45% lipid diet. 2 pairs were analyzed per group.

FIG. 14C shows the flow cytometry analysis of eWAT from 1 month old Csf1r^{MeriCreMer};Rosa^{LSL-tomato} mice pulse labelled with 4-hydroxy tamoxifen at E8.5. (Flow plot representative of n=4, from 2 litters).

FIG. 14D shows the fluorescent whole mount image of adipose tissue macrophages in the iWAT of 14 week old C57B1/6J mice fed a 45% lipid diet for 8 weeks (representative of n=15), CLS denotes crown-like structures.

FIG. 14E shows the quantification of Tim4⁺ and Tim4⁻ macrophage populations in eWAT, iWAT and mWAT by flow cytometry in C57B1/6J mice fed 10% (n=10), 45% (n=5), or 45%>10% (n=5) lipid diet.

FIG. 14F shows the mouse body weight (Each dot represents the mean ± SD, n=10 for 10% and 45%, n=5 for 45%->10%) of C57BL/6J mice fed the indicated diet.

FIG. 14G shows the weight of adipose tissues from mice fed the indicated diet as in FIG. 14E (n=10 for 10%, n=5 for 45%, and n=5 for 45%>10%).

FIG. 14H shows the flow cytometry quantification of the blood Ly6C^{hi} monocytes (n=10 for 10%, n=5 for 45%, and n=5 for 45%>10%).

FIG. 14I shows the insulin and glucose tolerance test in C57BL/6J mice (Each dot represents the mean ± SD, n=5) fed the indicated diet for 8 weeks and then switched to 10% fat diet. Blood glucose concentration from the glucose tolerance test was normalized to the initial blood glucose level (time =0) and then the area under the curve was measured (n=4). Each dot represents one area under the curve from one mouse. p values obtained by one-way ANOVA unless otherwise stated.

FIG. 15A shows a multidimensional scaling analysis of RNA sequencing performed on adipose tissue macrophages and blood Ly6C^{hi} monocytes sorted from C57B1/6J males fed 10%, 45%, or 45%→10% lipid diet (n=2).

FIG. 15B shows a heatmap of differentially expressed genes in the color-coded macrophage subsets and blood monocytes from C57B1/6J mice fed the indicated diet. Heatmap depicts z-scores.

FIG. 15C shows a heatmap depicting clusters of differentially expressed genes. Heatmap depicts z-scores.

FIG. 15D shows the counts per million (CPM) for each sample in the different clusters. Number above depicts the cluster number.

FIG. 15E shows tables depicting significantly expressed gene ontology categories for cluster 3, 4, 6, 7 and 8. Only the gene ontology categories with a -log(q-value) superior to 1 are represented.

FIG. 15F shows a violin plot representing the part of the variance explained by the difference between the populations, differences between the diets, different sorting dates, or quality of the samples.

FIG. 16A shows the expression of indicated genes, relative to Gapdh and Actinb (2⁻^{ΔCt}), as determined by qPCR in FACS-sorted WAT macrophage subsets, blood monocytes, and liver Kupffer cells in C57B1/6J fed a 10% or 45% lipid diet for 8 weeks (n=7 for adipose macrophages, n=9 for monocytes).

FIG. 16B shows the qPCR analysis of Bmp, Igf1, and Vegfb transcripts from the iWAT of C57BL/6J mice (n=9-17 for 10%, n=10-13 for 45%), Ccr2^{-/-} (n=5-6 for 10%, n=5-6 for 45%), PLX5622-treated mice (n=6-8 10% + PLX5622, n=7-8 45% + PLX5622) and and controls fed 10% or 45% lipid diet for 8 weeks, normalized to Gapdh. Each dot represents one mouse, p values obtained by one-way ANOVA with Tukey’s correction.

FIGS. 16C-16D show heatmap representations of expression in the different populations in 10% or 45% lipid diets (n=2) for genes associated with (FIG. 16C) M1 or (FIG. 16D) M2 polarization of macrophages (Shaul et al, Diabetes 2010). Each column is color-coded. Heatmap depicts z-scores.

FIG. 17A shows the quantification of CLS from the histological section of iWAT taken from Ccr2 ^{-/-} and littermate controls (n=3). Photograph of a Crown-like structure (CLS), denoted by asterisk, from perilipin stained paraffin embedded sections of iWAT from mice fed a 45% lipid diet.

FIG. 17B shows the glucose and insulin tolerance test on Ccr2 ^{-/-} and littermate control mice fed 10% (n=4) lipid diet or 45% (n=5) lipid diet for 8 weeks. Each dot represents the mean ± SD. p values obtained by t-test comparing the area under the curves for the indicated groups. Blood glucose concentration from the glucose tolerance test was normalized to the initial blood glucose level (time =0) and then the area under the curve was measured. Each dot represents one area under the curve from one mouse.

FIG. 17C shows the mouse body weight (Each dot represents the mean ± SD, n=5) of Ccr2 ^{-/-} mice and littermate controls fed 10% lipid diet or 45% lipid diet for 8 weeks.

FIG. 17D shows the perilipin staining of paraffin embedded iWAT section from Ccr2 ^{-/-} mice and littermate controls fed 10% lipid diet or 45% lipid diet for 8 weeks (n=4 for 10%, n=5 for 45% from 2 litters).

FIG. 17E shows the flow cytometry quantification of iWAT adipose tissue macrophages from 14-week-old C57B1/6J mice (n=9 for 10%, n=13 for 45% from 3 experiments) fed the indicated diet for 6-8 weeks and treated with PLX5622 (n=4 for 10% and 45%), a-CSF 1R blocking antibodies (n=5 for 45%), or vehicle/IgG control.

FIG. 17F shows the flow cytometry quantification of macrophages in the brain, liver, and kidney of C57B1/6J mice fed PLX5622 (n=5), i.p injected with anti-CSF1R (AFS98) antibodies (n=4-5), or vehicle control for one week (n=6-10).

FIG. 17G shows the qPCR analysis of Tnf and Il1b transcripts in the liver of mice fed 10% or 45% fat diet with the indicated treatment(n=4-6), normalized to Gapdh (2-ΔCt).

FIG. 17H shows the liver Triglycerides per mg of tissue in mice on the indicated diet (n=4-5). Representative H&E staining of livers from C57BL/6J mice on the indicated diets.

FIG. 17I shows the liver weight of C57B1/6J mice fed the indicated diet with the denoted treatment for 8 weeks(n=4-7).

FIG. 17J shows the total Liver Triglycerides in mice on the indicated diet (n=4-9).

FIG. 17K shows the glucose and insulin tolerance test on mice fed 10% lipid diet or 45% lipid diet with/without PLX5622 for 8 weeks (n=5). Blood glucose concentration from the glucose tolerance test was normalized to the initial blood glucose level (time =0) and then the area under the curve was measured (n=5). Each dot represents one area under the curve from one mouse.

FIG. 17L shows the mouse body weight over time fed the indicated diet (10% lipid diet group n=5, 45% lipid diet group n=7, 10% lipid diet group + PLX5622 n=5, 45% lipid diet group + PLX5622 n=10). Mean ± SD, p values at 14 weeks of age obtained by t-test.

FIG. 17M shows the cumulative food intake was measured gravimetrically for 5 days over the first week of treatment (n=8).

FIG. 17N shows the cumulative ambulatory activity was acquired using a laser matrix, each dot represents mean ± SD of n=8.

FIG. 17O shows the qPCR analysis of the indicated genes from the iBAT of mice fed 10% or 45% lipid diet for 8 weeks (n=4) , normalized to Gapdh. Each dot represents one mouse, p values are obtained by one-way ANOVA with Sidak’s correction unless otherwise stated.

FIG. 18A shows the mouse body weight (Each dot represents the mean ± SD, n=4) of Lepr^{-/-} mice fed 10% lipid diet + PLX5622 or 10% lipid diet for 8 weeks. p values at 12 weeks of age obtained by t-test.

FIG. 18B shows the qPCR analysis of Pdgfc transcripts in total iWAT (n=4), normalized to Gapdh (2^{-ΔCt}).

FIG. 18C shows the qPCR analysis of Tnf and Il1b transcripts in the liver of mice fed 10% fat diet with the indicated treatment (n=4), normalized to Gapdh (2^{-ΔCt}).

FIG. 18D shows the liver weight of Lepr^{-/-} mice fed the indicated diet for 8 weeks (n=4).

FIG. 18E shows the total Liver Triglycerides in mice on the indicated diet (n=4).

FIG. 18F shows the representative H&E staining of livers from mice on the indicated diets. Liver Triglycerides per mg of tissue in mice on the indicated diet (n=4).

FIG. 18G shows the qPCR analysis of Ucp1 and Dio2 in the iBAT mice treated with/without PLX5622 (n=5). Data is normalized to Gapdh for calculation of the 2^{-ΔC}.

FIG. 18H shows the cumulative ambulatory activity was acquired using a laser matrix, each dot represents mean ± SD of n=4. Each dot represents one mouse. p values obtained by t-test unless otherwise stated.

### DETAILED DESCRIPTION

It is to be appreciated that certain aspects, modes, embodiments, variations and features of the present technology are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. Eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. Eds (1996) Weir’s Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

### I. Definitions

The definitions of certain terms as used in this specification are provided below. Unless defined otherwise, all technical and scientific terms used herein generally have the same meaning as commonly understood by one of ordinary skill in the art to which this present technology belongs.

As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent, drug, or peptide to a subject includes any route of introducing or delivering to a subject a compound to perform its intended function. Administration can be carried out by any suitable route, including orally, intranasally, parenterally (intravenously, intramuscularly, intraperitoneally, or subcutaneously), intratumorally (e.g., in the case of administration to a tumor, such as a liposarcoma), or topically. In some embodiments, the therapeutic agents (including anti-PDGFcc, anti-Csf1r antibody, VEGFb peptide, PDGFcc peptide, etc.) of the present technology are administered by an intracoronary route or an intra-arterial route. Administration includes self-administration and the administration by another.

As used herein, the term “amino acid” is used to refer to any organic molecule that contains at least one amino group and at least one carboxyl group. Typically, at least one amino group is at the α position relative to a carboxyl group. The term “amino acid” includes naturally-occurring amino acids and synthetic amino acids, as well as amino acid analogs and amino acid mimetics that function in a manner similar to the naturally-occurring amino acids. Naturally-occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally-occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally-occurring amino acid. Amino acid mimetics refer to chemical compounds that have a structure that is different from the general chemical structure of an amino acid, but that functions in a manner similar to a naturally-occurring amino acid. Amino acids can be referred to herein by either their commonly known three letter symbols or by the one-letter symbols recommended by the IUPAC-IUB Biochemical Nomenclature Commission.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. As used herein, “antibodies” (includes intact immunoglobulins) and “antigen binding fragments” specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴ M⁻¹ greater or at least 10⁵ M⁻¹ greater than a binding constant for other molecules in a biological sample). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997.

As used herein, the term “conjugated” refers to the association of two molecules by any method known to those in the art. Suitable types of associations include chemical bonds and physical bonds. Chemical bonds include, for example, covalent bonds and coordinate bonds. Physical bonds include, for instance, hydrogen bonds, dipolar interactions, van der Waal forces, electrostatic interactions, hydrophobic interactions and aromatic stacking.

As used herein, an “antigen” refers to a molecule to which an antibody (or antigen binding fragment thereof) can selectively bind. The target antigen may be a protein, carbohydrate, nucleic acid, lipid, hapten, or other naturally occurring or synthetic compound. In some embodiments, the target antigen may be a polypeptide (e.g., including anti-PDGFcc, anti-Csf1r antibody, VEGFb peptide, PDGFcc peptide, etc.). An antigen may also be administered to an animal to generate an immune response in the animal.

The term “antigen binding fragment” refers to a fragment of the whole immunoglobulin structure which possesses a part of a polypeptide responsible for binding to antigen. Examples of the antigen binding fragment useful in the present technology include scFv, (scFv)₂, scFv-Fc, Fab, Fab′ and F(ab′)₂, but are not limited thereto.

As used herein, the term “biological sample” means sample material derived from living cells. Biological samples may include tissues, cells, protein or membrane extracts of cells, and biological fluids (e.g., ascites fluid or cerebrospinal fluid (CSF)) isolated from a subject, as well as tissues, cells and fluids present within a subject. Biological samples of the present technology include, but are not limited to, samples taken from breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, thymus, blood, hair, buccal, skin, serum, plasma, CSF, semen, prostate fluid, seminal fluid, urine, feces, sweat, saliva, sputum, mucus, bone marrow, lymph, and tears. Biological samples can also be obtained from biopsies of internal organs. Biological samples can be obtained from subjects for diagnosis or research or can be obtained from non-diseased individuals, as controls or for basic research. Samples may be obtained by standard methods including, e.g., venous puncture and surgical biopsy. In certain embodiments, the biological sample is an adipose tissue.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

An “isolated” or “purified” polypeptide or peptide is substantially free of cellular material or other contaminating polypeptides from the cell or tissue source from which the agent is derived, or substantially free from chemical precursors or other chemicals when chemically synthesized. For example, isolated including anti-PDGFcc, anti-Csf1r antibody, VEGFb peptide, PDGFcc peptide, etc., of the present technology would be free of materials that would interfere with diagnostic or therapeutic uses of the agent. Such interfering materials may include enzymes, hormones and other proteinaceous and nonproteinaceous solutes.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the term “gene” means a segment of DNA that contains all the information for the regulated biosynthesis of an RNA product, including promoters, exons, introns, and other untranslated regions that control expression.

As used herein, the terms “homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. A polynucleotide or polynucleotide region (or a polypeptide or polypeptide region) has a certain percentage (for example, at least 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art. In some embodiments, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Details of these programs can be found at the National Center for Biotechnology Information. Biologically equivalent polynucleotides are those having the specified percent homology and encoding a polypeptide having the same or similar biological activity. Two sequences are deemed “unrelated” or “non-homologous” if they share less than 40% identity, or less than 25% identity, with each other.

As used herein, “humanized” forms of non-human (e.g., murine) antibodies are chimeric antibodies which contain minimal sequence derived from non-human immunoglobulin. For the most part, humanized antibodies are human immunoglobulins in which hypervariable region residues of the recipient are replaced by hypervariable region residues from a non-human species (donor antibody) such as mouse, rat, rabbit or nonhuman primate having the desired specificity, affinity, and capacity. In some embodiments, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance such as binding affinity. Generally, the humanized antibody will comprise substantially all of at least one, and typically two, variable domains (e.g., Fab, Fab′, F(ab′)₂, or Fv), in which all or substantially all of the hypervariable loops correspond to those of a non-human immunoglobulin and all or substantially all of the FR regions are those of a human immunoglobulin consensus FR sequence although the FR regions may include one or more amino acid substitutions that improve binding affinity. The number of these amino acid substitutions in the FR are typically no more than 6 in the H chain, and in the L chain, no more than 3. The humanized antibody optionally may also comprise at least a portion of an immunoglobulin constant region (Fc), typically that of a human immunoglobulin. For further details, see Jones et al., Nature 321:522-525 (1986); Riechmann et al., Nature 332: 323-327 (1988); and Presta, Curr. Op. Struct. Biol. 2:593-596 (1992). See e.g., Ahmed & Cheung, FEBS Letters 588(2):288-297 (2014); Saxena & Wu, Frontiers in immunology 7: 580 (2016).

As used herein, the terms “identical” or percent “identity”, when used in the context of two or more nucleic acids or polypeptide sequences, refer to two or more sequences or subsequences that are the same or have a specified percentage of amino acid residues or nucleotides that are the same (i.e., about 60%, 65%, 70%, 75%, 80%, 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein)), when compared and aligned for maximum correspondence over a comparison window or designated region as measured using a BLAST or BLAST 2.0 sequence comparison algorithms with default parameters described below, or by manual alignment and visual inspection (e.g., NCBI web site). Such sequences are then said to be “substantially identical.” This term also refers to, or can be applied to, the complement of a test sequence. The term also includes sequences that have deletions and/or additions, as well as those that have substitutions. In some embodiments, identity exists over a region that is at least about 25 amino acids or nucleotides in length, or 50-100 amino acids or nucleotides in length.

As used herein, the term “intact antibody” or “intact immunoglobulin” means an antibody that has at least two heavy (H) chain polypeptides and two light (L) chain polypeptides interconnected by disulfide bonds. Each heavy chain is comprised of a heavy chain variable region (abbreviated herein as HCVR or V_{H}) and a heavy chain constant region. The heavy chain constant region is comprised of three domains, CHi, CH₂ and CH₃. Each light chain is comprised of a light chain variable region (abbreviated herein as LCVR or V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FRi, CDR₁, FR₂, CDR₂, FR₃, CDR₃, FR₄. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system.

As used herein, the terms “individual”, “patient”, or “subject” can be an individual organism, a vertebrate, a mammal, or a human. In some embodiments, the individual, patient or subject is a human.

The term “monoclonal antibody” as used herein refers to an antibody obtained from a population of substantially homogeneous antibodies, i.e., the individual antibodies comprising the population are identical except for possible naturally occurring mutations that may be present in minor amounts. For example, a monoclonal antibody can be an antibody that is derived from a single clone, including any eukaryotic, prokaryotic, or phage clone, and not the method by which it is produced. A monoclonal antibody composition displays a single binding specificity and affinity for a particular epitope. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. Monoclonal antibodies can be prepared using a wide variety of techniques known in the art including, e.g., but not limited to, hybridoma, recombinant, and phage display technologies. For example, the monoclonal antibodies to be used in accordance with the present methods may be made by the hybridoma method first described by Kohler et al., Nature 256:495 (1975), or may be made by recombinant DNA methods (See, e.g., U.S. Patent No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration. Pharmaceutically-acceptable carriers and their formulations are known to one skilled in the art and are described, for example, in Remington’s Pharmaceutical Sciences (20^{th} edition, ed. A. Gennaro, 2000, Lippincott, Williams & Wilkins, Philadelphia, PA.).

As used herein, “prevention” or “preventing” of a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

As used herein, the terms “polypeptide,” “peptide,” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, “specifically binds” refers to a molecule (e.g., an antibody or antigen binding fragment thereof) which recognizes and binds another molecule (e.g., an antigen), but that does not substantially recognize and bind other molecules. The terms “specific binding,” “specifically binds to,” or is “specific for” a particular molecule (e.g., a polypeptide, or an epitope on a polypeptide), as used herein, can be exhibited, for example, by a molecule having a K_{D} for the molecule to which it binds to of about 10⁻⁴ M, 10⁻⁵ M, 10⁻⁶ M, 10⁻⁷ M, 10⁻⁸M, 10⁻⁹ M, 10⁻¹⁰ M, 10⁻¹¹ M, or 10⁻¹² M. The term “specifically binds” may also refer to binding where a molecule (e.g., an antibody or antigen binding fragment thereof) binds to a particular polypeptide (e.g., a PDGFcc peptide or a Csf1r peptide), or an epitope on a particular polypeptide, without substantially binding to any other polypeptide, or polypeptide epitope.

As used herein, the terms “subject,” “individual,” or “patient” can be an individual organism, a vertebrate, a mammal, or a human.

“Treating”, “treat”, or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. For example, a subject is successfully “treated” for obesity, lipodystrophy, or cachexia, if, after receiving a therapeutic amount of the anti-PDGFcc antibody, anti-Csf1r antibody, VEGFb peptide, PDGFcc peptide, etc., of the present technology according to the methods described herein, the subject shows observable and/or measurable increase in lipid storage by adipocytes, when treating lipodystrophy and cachexia, or the subject shows observable and/or measurable decrease in lipid storage by adipocytes, when treating or preventing lipid accumulation in adipose tissue or treating obesity.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

### II. Introduction

Landmark studies from the 1980’s onwards have established that the activation and recruitment of monocyte/macrophages in obese adipose tissue and peripheral tissues such as the liver cause inflammation, insulin resistance, and type 2 diabetes via the production of inflammatory cytokines such as TNF. However, macrophages are also present both in lean and adipose tissue. The present technology is based, in part, on the discovery that the adipose tissue resident macrophages, but not HSC-derived monocyte/macrophages, are required for lipid storage in visceral and subcutaneous white adipose tissue via local production of PDGF/VEGF-family growth factors. The same mechanism controls lipid storage in the Drosophila fat body and adipocyte hypertrophy in obese adult mice.

The present technology relates to the discovery of targeting PDGFcc as a means by which to ameliorate abnormal lipid storage in adipose tissue. In some embodiments, the disclosure of the present technology relates to the use of anti-PDGFcc antibodies to target adipocyte hypertrophy in a subject in need thereof. While other drugs aimed at treating inflammation associated with abnormal lipid accumulation, such as obesity, can have systemic effects, the use of anti-PDFGcc antibodies as described herein avoids these off-target effects by targeting adipose tissue fat storage. The disclosure of the present technology demonstrates that macrophages from different developmental origins exert distinct functions within the same tissue. The discovery of a macrophage-adipocyte functional unit that controls energy storage in the specialized fat tissues of metazoans is described herein. The disclosure of the present technology also relates to the characterization of signaling components of the macrophage-adipocyte functional unit that are relevant to the treatment of metabolic diseases associated with abnormal lipid accumulation, including lipodystrophy and cachexia, as well as the disorders featuring increased lipid accumulation and/or lipid storage in adipose tissue, such as hyperlipidemia, and obesity.

### III. PDGFcc

Platelet-derived growth factors (PDGFs), which include PDGFA, PDGFB, PDGFC, and PDGFD, play crucial roles in the regulation of a wide range of biological processes, including cell proliferation, survival, migration, angiogenesis, tissue remodeling, and organogenesis (e.g., the development of axial skeleton, palate, teeth, and the cardiovascular system). PDGFs exert their biological functions by binding to and activating two receptor TKs (PDGF receptor alpha [PDGFRA or PDGFRα] and PDGFRB).

PDGF-CC is a member of the PDGF family, which is biosynthesized as a precursor protein, which includes a growth factor domain (GFD) and an N-terminal CUB (homology to complement components Clr/Cls, Uegf, BMP1) domain. The CUB domain sterically blocks the receptor binding surface in the GFD and has to be proteolytically removed in order to release the GFD dimer and to produce a mature PDGF-CC protein (hereinafter “PDGFcc”), which can bind to its receptor, PDGFRα (PDGFRA/A homodimers). Fredriksson et al., The EMBO journal 23(19): 3793-802 (2004). It has been reported that PDGFcc binds to PDGFRA/A homodimers with high affinity but fails to interact with the PDGFRB/B homodimers. Gilbertson et al., J Biol Chem. 276(29): 27406-14 (2001).

An exemplary human PDGFC precursor (NCBI Reference Sequence: NP_057289.1, SEQ ID NO: 1) has the following sequence:

MSLFGLLLLTSALAGQRQGTQAESNLSSKFQFSSNKEQNGVQDPQHERIITVSTNGSIHSPRFPHTYPRNTVLVWRLVAVEENVWIQLTFDERFGLEDPEDDICKYDFVEVEEPSDGTILGRWCGSGTVPGKQISKGNQIRIRFVSDEYFPSEPGFCIHYNIVMPQFTEAVSPSVLPPSALPLDLLNNAITAFSTLEDLIRYLEPERWQLDLEDLYRPTWQLLGKAFVFGRKSRVVDLNLLTEEVRLYSCTPRNFSVSIREELKRTDTIFWPGCLLVKRCGGNCACCLHNCNECQCVPSKVTKKYHEVLQLRPKTGVRGLHKSLTDVALEHHEECDCVCRGSTGG

This protein undergoes a proteolytic removal of the N-terminal CUB domain, releasing the core domain that is necessary for unmasking the receptor-binding epitopes of the core domain. Cleavage after basic residues in the hinge region (region connecting the CUB and growth factor domains), by plasminogen activator tissue (PLAT) and plasminogen (PLG), gives rise to the receptor-binding form. See UniProtKB - Q9NRA1 (PDGFC_HUMAN). The cleavage sites are indicated by boldface-underlined font.

An exemplary PDGFcc protein has the following sequence (SEQ ID NO: 2):

VVDLNLLTEEVRLYSCTPRNFSVSIREELKRTDTIFWPGCLLVKRCGGNCACCLHNCNECQCVPSKVTKKYHEVLQLRPKTGVRGLHKSLTDVALEHHEECDCVCRGSTGG

Recombinant PDGFcc can be produced using conventional techniques in molecular biology, protein biochemistry, cell biology, immunology, microbiology and recombinant DNA. See, e.g., Sambrook and Russell eds. (2001) Molecular Cloning: A Laboratory Manual, 3rd edition; the series Ausubel et al. Eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. Eds (1996) Weir’s Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

Recombinant PDGFcc is also commercially available from vendors, including R&D Systems, Inc. (Catalog No. 1687-CC-025) and Novus Biologicals (Cat. No. NBP2-36517).

### IV. PDGFcc Antagonists

Any PDGFcc antagonists that inhibit PDGFcc signaling may be used in the methods disclosed herein. In some embodiments, the PDGFcc antagonists inhibit PDGFcc itself. In alternate embodiments, the PDGFcc antagonists inhibit signaling by PDGFRα (PDGFRA/A homodimers). Non-limiting examples of PDGFcc antagonists include anti-PDGFcc antibodies. Anti-PDGFcc antibodies include monoclonal antibodies, polyclonal antibodies, humanized antibodies, chimeric antibodies, recombinant antibodies, etc., as well as antibody fragments. Non-limiting examples of anti-PDGFcc antibodies suitable for the methods disclosed herein include 6B3, 9A5, 11F5, 19A1, 19B1, 19C7 and 19D1 (Li et al., PLoS ONE 13(7): e0201089 (2018); Zeitelhofer et al., PLoS ONE 13(7): e0200649 (2018)). Non-limiting examples of anti-PDGFcc polyclonal antibodies suitable for the methods disclosed herein include human PDGF-C Antibody (R&D Systems, Cat. No. AF1560), affinity-purified rabbit IgG against human core PDGFCC (Su et al., Nat Med. 14(7): 731-737 (2008)). Examples of PDGFcc antagonists and/or anti-PDGFcc antibodies are disclosed in US Pat. Publication Nos. 2006/0104978 and 2005/0282233. Small molecules, including tyrosine kinase inhibitors (TKIs), can be used to prevent the intracellular domain phosphorylation of PDGFR-α after ligand binding. Tyrosine kinase inhibitors with high specificity for PDGFR-α/β inhibition include imatinib, sunitinib, sorafenib, pazopanib, nilotinib, dasatinib, and masitinib.

### V. Csflr

Colony stimulating factor 1 receptor (CSF1R), also known as macrophage colony-stimulating factor receptor (M-CSFR), and CD115 (Cluster of Differentiation 115), is a cell-surface protein encoded, in humans, by the CSF1R gene (known also as c-FMS). CSF1R is the receptor for a cytokine called colony stimulating factor 1.

### VI. CCr2

C-C chemokine receptor type 2 (CCR2 or CD192) is a protein encoded by the CCR2 gene. CCR2 is a CC chemokine receptor. This gene encodes two isoforms of a receptor for monocyte chemoattractant protein-1 (CCL2), a chemokine that specifically mediates monocyte chemotaxis. Monocyte chemoattractant protein-1 is involved in monocyte infiltration in inflammatory diseases such as rheumatoid arthritis as well as in the inflammatory response against tumors. Any CCR2 antagonists or anti-CCR2 antibodies that inhibit CCR2 signaling may be used in the methods disclosed herein. Exemplary, non-limiting examples include, 15a, PF-04136309 (PF-6309), CCX872, or those disclosed in Struthers & Pasternak Curr. Top. Med. Chem. 10(13):1278-1298 (2010).

### VII. Metabolic Diseases Associated With Abnormal Lipid Accumulation

### A. Lipodystrophy

Lipodystrophy is a heterogeneous group of rare acquired and inherited disorders characterized by inability to produce and maintain healthy fat tissue. Lipodystrophy can be caused by metabolic abnormalities due to genetic issues. It is often characterized by insulin resistance and associated with metabolic syndrome.

HIV-associated lipodystrophy is a condition characterized by loss of subcutaneous fat associated with infection with HIV, with fat loss in face, buttocks, arms, and legs. There is evidence indicating both that it can be caused by anti-retroviral medications and that it can be caused by HIV infection in the absence of anti-retroviral medication. Giralt et al., Antivir Ther. 11(6): 729-40 (2006).

Lipodystrophy can also be a possible side effect of antiretroviral drugs, most commonly seen in patients treated with thymidine analogue nucleoside reverse transcriptase inhibitors like zidovudine (AZT) and stavudine (d4T). Lipodystrophy is also associated with HIV-1 protease inhibitors. Interference with lipid metabolism is postulated as pathophysiology. Also, the development of lipodystrophy is associated with specific nucleoside reverse transcriptase inhibitors (NRTI). Mitochondrial toxicity is postulated to be involved in the pathogenesis associated with NRTI.

### B. Hyperlipidemia

Hyperlipidemia is abnormally elevated levels of lipids or lipoproteins in the blood. Hyperlipidemias are divided into primary and secondary subtypes. Primary hyperlipidemia is usually due to genetic causes (such as a mutation in a receptor protein), while secondary hyperlipidemia arises due to other underlying causes such as diabetes. Lipid and lipoprotein abnormalities are common in the general population and are regarded as modifiable risk factors for cardiovascular disease due to their influence on atherosclerosis. In addition, some forms of hyperlipidemia may predispose the subject to acute pancreatitis.

### C. Cachexia

Cancer-associated cachexia is a disorder characterized by specific losses of muscle and adipose tissue. Cachexia is driven by metabolic changes, including elevated energy expenditure, excess catabolism, and inflammation. Adipose tissue cachexia is associated with cancers of the pancreas, oesophagus, stomach, lung, liver and bowel.

Management and treatment of cachexia includes nutritional interventions, including, but not limited to, supplementation with glutamine, L-carnitine, and amino acid supplementation (e.g., leucine metabolite, L-glutamine, and L-arginine). Liquid nutritional supplementation is also recommended for patients with cachexia.

Various agents have been administered in attempts to retard or halt progressive cachexia in cancer patients. In addition, a number of agents are currently being studied in animals. These agents include, but are not limited to: corticosteroids, such as prednisolone, methylprednisolone, and dexamethasone; progestational agents, such as megestrol acetate and medroxyprogesterone; cannabinoids (dronabinol); serotonin antagonists, such as cyproheptadine; prokinetic agents, such as metoclopramide and cisapride; anabolic steroids, such as nandrolone decanoate and fluoxymesterone; testosterone and its derivatives, such as oxandrolone and enobosarm; ghrelin (anamorelin); inhibitors of phosphoenolpyruvate carboxykinase, such as hydrazine sulfate; methylxanthine analogs, such as pentoxifylline and lisofylline; thalidomide; cytokines and anticytokines, such as anti-IL-6 antibody and IL-12; branched-chain amino acids; eicosapentaenoic acid; inhibitors of prostaglandin synthesis, such as indomethacin and ibuprofen; celecoxib; psychiatric drugs, such as mirtazapine and olanzapine; hormones, such as melatonin; and β₂-adrenocreceptor agonists, such as clenbuterol.In some embodiments, the present technology relates to a method for treating cachexia in a subject in need thereof, comprising administering to the subject an effective amount of PDGFcc or a PDGFcc agonist, an active fragment, or homolog thereof. In some embodiments, the methods of the present technology further comprise administering PDGFcc or PDGFcc agonist, an active fragment or homolog thereof separately, sequentially, or simultaneously with at least one: CCR2 antagonist, anti-CCR2 antibody, VEGFb, an active fragment, or a homolog thereof; or one or more additional therapeutic agents selected from the group consisting of: corticosteroids, such as prednisolone, methylprednisolone, and dexamethasone; progestational agents, such as megestrol acetate and medroxyprogesterone; cannabinoids (dronabinol); serotonin antagonists, such as cyproheptadine; prokinetic agents, such as metoclopramide and cisapride; anabolic steroids, such as nandrolone decanoate and fluoxymesterone; testosterone and its derivatives, such as oxandrolone and enobosarm; ghrelin (anamorelin); inhibitors of phosphoenolpyruvate carboxykinase, such as hydrazine sulfate; methylxanthine analogs, such as pentoxifylline and lisofylline; thalidomide; cytokines and anticytokines, such as anti-IL-6 antibody and IL-12; branched-chain amino acids; eicosapentaenoic acid; inhibitors of prostaglandin synthesis, such as indomethacin and ibuprofen; celecoxib; psychiatric drugs, such as mirtazapine and olanzapine; hormones, such as melatonin; and β₂-adrenocreceptor agonists, such as clenbuterol.

### VIII. Liposarcoma Debulking

Debulking is the reduction of as much of the bulk (e.g., volume) of a tumor as possible. It is typically achieved by surgical removal (surgical debulking). Liposarcomas (malignant adipocytic tumors that store lipids) involve the peritoneal cavity in approximately 30% of the cases. The treatment for such liposarcomas is surgery. Abdominal liposarcomas are associated with a high local recurrence rate. Re-operation is the only effective treatment for recurrent abdominal liposarcoma (RAL). For those who are not amenable to complete radical resection, debulking resection may relieve symptoms, reduce complications, and increase the life span.

In some embodiments, the present technology relates to a method for debulking liposarcoma as a pre-operative treatment with a PDGFcc antagonist, an anti-PDGFcc antibody, or a fragment thereof, to decrease the tumor size and facilitate its surgical removal.

### IX. Modes of Administration and Effective Dosages

Any method known to those in the art for contacting a cell, organ or tissue with an immunoglobulin-related composition may be employed. Suitable methods include in vitro, ex vivo, or in vivo methods. In vivo methods typically include the administration of PDGFcc, an anti-PDGFcc antagonist, an anti-PDGFcc antibody or a fragment thereof, such as those described above, to a mammal, suitably a human. When used in vivo for therapy, the PDGFcc, the anti-PDGFcc antagonist, the anti-PDGFcc antibody or a fragment thereof are administered to the subject in effective amounts (i.e., amounts that have desired therapeutic effect). The dose and dosage regimen will depend upon the degree of the disease symptoms in the subject, the characteristics of the particular the PDGFcc, the anti-PDGFcc antagonist, the anti-PDGFcc antibody or a fragment thereof used, e.g., its therapeutic index, the subject, and the subject’s history.

The effective amount may be determined during pre-clinical trials and clinical trials by methods familiar to physicians and clinicians. An effective amount of an immunoglobulin-related composition useful in the methods may be administered to a mammal in need thereof by any of a number of well-known methods for administering pharmaceutical compounds. The immunoglobulin-related composition may be administered systemically or locally.

The PDGFcc, the anti-PDGFcc antagonist, the anti-PDGFcc antibody or a fragment thereof described herein can be incorporated into pharmaceutical compositions for administration, singly or in combination, to a subject for the treatment or prevention of a disorder described herein. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose via ls made of glass or plastic. For convenience of the patient or treating physician, the dosing formulation can be provided in a kit containing all necessary equipment (e.g., via ls of drug, vials of diluent, syringes and needles) for a treatment course (e.g., 7 days of treatment).

In some embodiments, the PDGFcc, the anti-PDGFcc antagonist, the anti-PDGFcc antibody or a fragment thereof of the present technology is administered by a parenteral route. In some embodiments, the antibody or antigen binding fragment thereof is administered by a topical route.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

The immunoglobulin-related compositions described herein can include a carrier, which can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, isotonic agents are included, for example, sugars, polyalcohols such as mannitol, sorbitol, or sodium chloride in the composition. Prolonged absorption of the injectable compositions can be brought about by including in the composition an agent which delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the immunoglobulin-related compositions of the present technology can be delivered in the form of an aerosol spray from a pressurized container or dispenser which contains a suitable propellant, e.g., a gas such as carbon dioxide, or a nebulizer. Such methods include those described in U.S. Pat. No. 6,468,798.

Systemic administration of an immunoglobulin-related composition of the present technology as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

An immunoglobulin-related composition of the present technology can be formulated in a carrier system. The carrier can be a colloidal system. The colloidal system can be a liposome, a phospholipid bilayer vehicle. In one embodiment, the therapeutic immunoglobulin-related composition is encapsulated in a liposome while maintaining structural integrity. As one skilled in the art would appreciate, there are a variety of methods to prepare liposomes. (See Lichtenberg et al., Methods Biochem. Anal., 33:337-462 (1988); Anselem et al., Liposome Technology, CRC Press (1993)). Liposomal formulations can delay clearance and increase cellular uptake (See Reddy, Ann. Pharmacother., 34(7-8):915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the PDGFcc, the anti-PDGFcc antagonist, the anti-PDGFcc antibody or a fragment thereof can be embedded in the polymer matrix, while maintaining protein integrity. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy et al.), U.S. Pat. Nos. 5,674,534 and 5,716,644 (both to Zale et al.), PCT publication WO 96/40073 (Zale et al.), and PCT publication WO 00/38651 (Shah et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the PDGFcc, the anti-PDGFcc antagonist, the anti-PDGFcc antibody or a fragment thereof are prepared with carriers that will protect the PDGFcc, the anti-PDGFcc antagonist, the anti-PDGFcc antibody or a fragment thereof against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The PDGFcc, the anti-PDGFcc antagonist, the anti-PDGFcc antibody or a fragment thereof can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4(3):201-9 (1994); and Gregoriadis, “Engineering Liposomes for Drug Delivery: Progress and Problems,” Trends Biotechnol., 13(12):527-37 (1995). Mizguchi et al., Cancer Lett., 100:63-69 (1996), describes the use of fusogenic liposomes to deliver a protein to cells both in vivo and in vitro.

Dosage, toxicity and therapeutic efficacy of the PDGFcc, the anti-PDGFcc antagonist, the anti-PDGFcc antibody or a fragment thereof can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. In some embodiments, the PDGFcc, the anti-PDGFcc antagonist, the anti-PDGFcc antibody or a fragment thereof exhibit high therapeutic indices. While the PDGFcc, the anti-PDGFcc antagonist, the anti-PDGFcc antibody or a fragment thereof that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies within a range of circulating concentrations that include the ED50 with little or no toxicity. The dosage may vary within this range depending upon the dosage form employed and the route of administration utilized. For any the PDGFcc, the anti-PDGFcc antagonist, the anti-PDGFcc antibody or a fragment thereof used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Typically, an effective amount of the PDGFcc, the anti-PDGFcc antagonist, the anti-PDGFcc antibody or a fragment thereof, sufficient for achieving a therapeutic or prophylactic effect, range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example, dosages can be 1 mg/kg body weight or 10 mg/kg body weight every day, every two days or every three days or within the range of 1-10 mg/kg every week, every two weeks or every three weeks. In one embodiment, a single dosage of an immunoglobulin-related composition ranges from 0.001-10,000 micrograms per kg body weight. In one embodiment, the PDGFcc, the anti-PDGFcc antagonist, the anti-PDGFcc antibody or a fragment thereof concentrations in a carrier range from 0.2 to 2000 micrograms per delivered milliliter. An exemplary treatment regime entails administration once per day or once a week. In therapeutic applications, a relatively high dosage at relatively short intervals is sometimes required until progression of the disease is reduced or terminated, and until the subject shows partial or complete amelioration of symptoms of disease. Thereafter, the patient can be administered a prophylactic regime.

In some embodiments, a therapeutically effective amount of an immunoglobulin-related composition of the present technology may be defined as a concentration of an immunoglobulin-related composition at the target tissue of 10⁻¹² to 10⁻⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue. In some embodiments, the doses are administered by single daily or weekly administration, but may also include continuous administration (e.g., parenteral infusion or transdermal application). In some embodiments, the dosage of the immunoglobulin-related compositions of the present technology is provided at a “low,” “mid,” or “high” dose level. In one embodiment, the low dose is provided from about 0.0001 to about 0.5 mg/kg/h, suitably from about 0.001 to about 0.1 mg/kg/h. In one embodiment, the mid-dose is provided from about 0.01 to about 1.0 mg/kg/h, suitably from about 0.01 to about 0.5 mg/kg/h. In one embodiment, the high dose is provided from about 0.5 to about 10 mg/kg/h, suitably from about 0.5 to about 2 mg/kg/h.

For example, a therapeutically effective amount of the anti-PDGFcc antagonist or the anti-PDGFcc antibody may partially or completely alleviate one or more symptoms of obesity, including lipid storage in adipose tissue, including adipocyte hypertrophy, and increased body weight, without modifying food intake, blood glucose levels, insulin levels, glucose tolerance, etc., and without causing ectopic storage of lipids (hepatosteatosis), which could be further controlled by a combination of PDGFcc antagonist or the anti-PDGFcc antibody with a CCR2 antagonist or anti-CCR2 antibody which limits hepatosteatosis, insulin resistance and glucose tolerance.

For example also, in the case of lipodystrophy and cachexia, a therapeutically effective amount of the PDGFcc or a PDGFcc agonist, may prevent loss of subcutaneous fat or increase fat storage in said fat, without modifying food intake, blood glucose levels, insulin levels, glucose tolerance, etc., which could be further controlled by a combination with a CCR2 antagonist or anti-CCR2 antibody to further prevent ectopic storage of lipids (hepatosteatosis), insulin resistance and glucose tolerance.

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance present methods can be any mammal, including, for example, farm animals, such as sheep, pigs, cows, and horses; pet animals, such as dogs and cats; laboratory animals, such as rats, mice and rabbits. In some embodiments, the mammal is a human.

### EXAMPLES

The present technology is further illustrated by the following Examples, which should not be construed as limiting in any way. Those of skill in the art will readily recognize a variety of non-critical parameters that could be changed or modified to yield essentially the same or similar results. The examples should in no way be construed as limiting the scope of the present technology, as defined by the appended claims.

### Example 1: Materials and Methods

Mice and diets. All experiments on mice were realized in accordance with an animal license issued by the Institutional Review Board (IACUC 15-04-006) from MSKCC. All mice were maintained under SPF conditions. C57B1/6J male mice were purchased from Jackson laboratories. Rosa26^{LSL-Tomato} (Stock No: 007908), Rosa26^{LSL-}^{YFP} (Stock No:006148), Rosa26^{mTmG} (Stock No: 007576) and Lepr^{-/-}(db/db) (herein referred to as Lepr^{-/-}; Stock No: 000697) mice were purchased from Jackson laboratories and bred in house. Csf1r^{Cre}, Csf1r^{MeriCreMer}, and Csf1r^{fl/fl} mice were obtained from J W. Pollard (The University of Edinburgh), Cx3cr1^{GFP/+} mice were obtained from Drs. Dan Littman (NYU Skirball institute), Flt3^{Cre} mice were obtained from Thomas Boehm (Max Planck institute) and Tnfrsf11a^{Cre} mice were obtained from Y. Kobayashi (Matsumoto Dental University). Ccr2^{-/-} mice were obtained from IF. Charro (UCSF) Csf1r^{-/-} mice were backcrossed to FVB/NJ for more than 10 generations. Mice were fed ad libitum a rodent irradiated diet containing 45% kcal from lipids (Research Diets Inc., reference D12451i) or 10% kcal from lipids (Research Diets Inc., reference D12450hi) for 8 weeks. In mice supplemented with the CSF-1R inhibitor PLX5622 (Plexxikon), the aforementioned diets were impregnated with 1200 mg of inhibitor per kg of food. In the experiment named 45%>10% the mice were fed 45% lipid diet for 8 weeks then 10% lipid diet for 2 weeks and compared to mice fed 10% lipid diet for 10 weeks. Mice were weighed weekly. Tnfrsf11a^{Cre}; Csflr^{f/f} and Csf1r^{Cre}; Csf1r^{f/f} mice are osteopetrotic and as such lack teeth. To prevent confounding systemic effects due to nutritional intake problems, the Tnfrsf11a^{Cre}; Csf1r^{f/f} Csf1r^{Cre}; Csf1r^{f/f} and control littermates were fed a nutritionally fortified water gel. The nutritional gels were replaced every 12 hours.

Parabiosis and Fate Mapping. For parabiosis experiments, 6 weeks old congenic CD45.1 and CD45.2 were surgically joined. Skin was incised from elbow to knee, forelimbs and hind limbs were joined with nylon suture, and then skin incisions were sutured with stainless steel clips. The mice were fed a sulfatrim diet for 2 weeks following the surgery and before starting 10% lipid diet or 45% lipid diet for 8 weeks. Chimerism in Tim4+ and Tim4- macrophage populations, as well as in liver Kupffer cells and blood lymphocytes and monocytes was assessed by flow cytometry.

Fate mapping experiments in Csf1r^{MeriCreMer} mice were performed as described; in briefCsf1r^{MeriCreMer} females were crossed to Rosa26^{LSL-tomato} or Rosa26^{LSL-YFP} males and injected intraperitoneally at 8.5 days post coitum with 75 mg per kg of body weight of 4-hydroxytamoxifen (Sigma) plus 37.5 mg progesterone (Sigma) per kg of body weight to counteract possible abortion induction. Tomato or YFP expression was then assessed in adult progeny by flow cytometry.

Glucose and insulin tolerance tests were performed as described; in brief, for the glucose tolerance test, the mice were fasted for 4h before experiment, but had water ad libitum. The weight and glycaemia were measured, and the mice were injected intraperitoneally with 2 g glucose (Thermo Scientific) per kg of body weight. Following the glucose injection, glycemia was measured every 30 min for 2 hours. For the insulin tolerance test, the mice were fasted for 4h before experiment, but had water ad libitum. The weight and the glycaemia were measured, and the mice were injected with 0.25U insulin (Sigma) per kg of body weight. Following the glucose injection, glycemia was measured every 30 min for 2 hours. To measure the glycaemia, the tail of the animals was pricked with a 27G needle and a drop of blood was placed on the glucometer (ACCU-CHECK AVIVA, Roche).

For tissue collection, mice were sacrificed by overdose of anesthetic, with intraperitoneal injection of ketamine (50 mg/kg), xylazine (10 mg/kg) and acepromazine (1.7 mg/kg).

Once the withdrawal reflexes (paw) were abolished, intracardiac puncture was performed for blood collection, with an EDTA (100 mM, Sigma) coated syringe (26G needle, 1 mL syringe, BD). The heart was then perfused with 10 mL PBS at room temperature. The mice were then sacrificed by cervical dislocation and the tissues collected.

Mice Antibody Treatment. Anti-CSF1R (clone AFS98, BioXcell) was administered intraperitoneally at 50 mg/kg in 100 µL PBS, and anti-PDGF-C (AF1447 R&D system) at 10 µg/mouse in 100 µL PBS. Antibodies were administrated every 3 days from the start of high fat diet. Mice treated with anti-CSF1R antibody also received an injection 3 days prior to high fat diet.

Metabolic analysis of Leptin receptor deficient mice, PLX5622-treated mice, anti-PDGFcc treated mice, and control C57Bl/6J mice. Animals were individually housed in a temperature controlled Promethion Metabolic Screening System (Sable Systems International, NV). Food intake was acquired gravimetrically and ambulatory activity was acquired using a laser matrix. Mice were acclimated to this environment on a 12 hr light/dark cycle for 48 hr before the indicated length of recording (24 hr to 120 hr) period began. Fecal bomb calorimetry was performed on feces collected during the last day of metabolic cage recording period, dehydrated in an oven at 60C for 48 hr, then combusted in technical duplicates with a Parr 6725 Semimicro Calorimeter to determine gross caloric intake.

Infrared imaging of mice. Infrared images were taken on the last day of metabolic analysis using a FLIR T430sc Infrared Camera. These images were into RAW files and then analyzed in AMIDE. A box was drawn over region of interest (ROI); interscapular (70×30), inguinal (35×35) and dorsal. The 10% top warmest pixels were used for interscapular and inguinal quantifications. The 20% top warmest pixels were used for dorsal quantifications. The variance refers to the variance of the voxels in the ROI.

Triglyceride Measurement for Mice. To measure triglycerides, ~ 100 mg of mouse liver was homogenized in 100 µl of PBS + 0.05% Tween 20. Triglyceride was then measured using the free glycerol reagent (Sigma; F6428) as described in the manufacturer’s protocol and normalized to the liver weight.

Cell Suspension Preparation for Flow Cytometry and Flow Cytometry Analysis. For the blood, red blood cells were lysed twice in ACK lysis buffer. Adipose tissue was collected in PBS, incubated for 20 min at 37° C. in collagenase II at 2 mg/mL (Sigma) in PBS supplemented with 0.25% BSA (Thermo Scientific) and 5mM CaCl₂ (Sigma) under agitation, before mechanical disruption with a 10 mL pipette. Cell suspensions were centrifuged for 10 min at 500 g. Liver, brain, and kidney samples were digested for 30 min at 37° C. in PBS containing 1 mg/ml of collagenase D (Roche), 100 U/ml DNaseI (Sigma), 2.4 mg/ml of dispase (Invitrogen) and 3% fetal calf serum (FCS, Invitrogen). Once processed, all samples were resuspended in FACS buffer (PBS, 0.5% BSA and 2 mm EDTA) containing anti-mouse CD16/32 (FcRIII/II, Biolegend Cat#: 101302) and anti-mouse CD16.2 (Biolegend Cat#: 149502) for 10 min and stained with antibody mixes for 30 min on ice. The list of antibodies used can be found in Table 1. After 2 washes with FACS buffer, samples were incubated with 2 µM Hoechst 33342 (Thermo Scientific) just prior to analysis using LSR Fortessa X-20 (BD bioscience) or sorting on an ARIA III (BD bioscience). The number of cells per gram of tissue was determined using a cell counter (GUAVA easyCyte HT).

TABLE 1

Antibodies Used for Whole Mount Imaging

| Antigen / marker | Clone | Fluorochrome | Provider | Dilution |
|---|---|---|---|---|
| Primary Antibodies | | | | |
| F4/80 | BM8 | eF570 or ef450 | eBioscience | 1/200 |
| CD11c | HL3 | eF450 | eBioscience | 1/100 |
| Tim4 | RMT4-54 | AF647 or PE | Biolegend | 1/200 |
| I-A/I-E | M5/114.15.2 | eF450 | BD Pharmingen | 1/100 |
| Lipidtox | - | Equivalent:AF647 | LifeTechnologies | |
| Bodipy | - | Equivilent:AF488 | LifeTechnologies | |
| Perilipin | - | AF488 | R&D System | 1/100 |
| PDGFRα | APA5 | APC | Biolegend | 1/100 |
| GFP | FM264G | AF488 | Biolegend | 1/100 |
| CD31 | MEC13.3 | AF647 | Biolegend | 1/100 |
| PDGFcc | | AF1560 | R&D System | 101 µg/ml |
| Secondary Antibodies | | | | |
| goat anti-rabbit | | AF647 | Invitrogen | 1/200 |
| goat anti-rat | | AF488 | Invitrogen | 1/200 |

Flow cytometry data were analyzed with Flow Jo 9.9. For t-SNE analysis of the adipose tissue stromal vascular fraction, FCS files from eWAT of 10%, 45% and 45%>10% lipid diet fed mice were concatenated. t-SNE algorithm, from Flow Jo 9.9, was used on singlets (determined by FSC-A, SSC-A gate, DAPI- live cells and FSC-W, FSC-A) from the concatenated FCS file created, with 1000 iterations, perplexity at 20 and Theta at 0.5. All channels with antibody staining were considered as well as FSC-A and SSC-A. Expression of the markers expressed by each cluster was performed after the separation of the concatenated samples.

TABLE 2

Antibodies Used for Flow Cytometry

| Antigen / marker | Clone | Fluorochrome | Provider | Dilution |
|---|---|---|---|---|
| CD45 | 30-F11 | APC-eF780 | eBioscience | 1/100 |
| CD45.1 | A20 | eF450 | eBioscience | 1/100 |
| CD45.2 | 104 | APC eF780 | eBioscience | 1/100 |
| CD3 | 1452-C11 | BV711 | BD Pharmingen | 1/200 |
| CD3 | 145-2C11 | APC Cy7 | BD Pharmingen | 1/200 |
| CD19 | 1D3 | BV711 | BD Pharmingen | 1/200 |
| NKp46 | 29A1.4 | BV711 | BD Pharmingen | 1/200 |
| NKp46 | 29A1.4 | AF647 | BD Pharmingen | 1/200 |
| SiglecF | E50-2440 | PE | BD Pharmingen | 1/200 |
| SiglecF | E50-2440 | BV711 | BD Pharmingen | 1/200 |
| Ly6G | 1A8 | PE | BD Pharmingen | 1/200 |
| Ly6G | 1A8 | BV711 | BD Pharmingen | 1/200 |
| Ly6C | HK1.4 | BV421 | Biolegend | 1/200 |
| Ly6C | HK1.4 | FITC | Biolegend | 1/200 |
| F4/80 | BM8 | BV605 | Biolegend | 1/200 |
| F4/80 | BM8 | eF450 | eBioscience | 1/100 |
| F4/80 | BM8 | BV605 | eBioscience | 1/100 |
| CD11b | M1/70 | PE Cy7 | eBioscience | 1/400 |
| Tim4 | RMT4-54 | AF647 | Biolegend | 1/200 |
| Tim4 | RMT4-54 | PE | Biolegend | 1/200 |
| CD206 | Mr5d3 | APC | BD Pharmingen | 1/200 |
| MertK | 2B 10C42 | PE | Biolegend | 1/200 |
| CD64 | X54-5/7.1 | FITC | Biolegend | 1/200 |
| CD11c | HL3 | BV421 | BD Pharmingen | 1/100 |
| CD11c | HL3 | FITC | BD Pharmingen | 1/100 |
| I-A/I-E | M5/114.15.2 | AF700 | Biolegend | 1/200 |
| CD115 | AFS98 | BV605 | Biolegend | 1/200 |
| DAPI | - | - | Invitrogen | 1/10000 |
| Hoechst 33258 | - | - | Invitrogen | 1/10000 |

Preparation of Libraries for RNA Sequencing and Analysis. To optimize cell viability, decrease cell activation and improve library quality, a low input RNA sequencing strategy was adopted. Libraries were prepared on 200 cells, reducing the sorting time, and flavopiridol (Sigma) was added during the sample preparation to inhibit transcription. Blood was processed as described above, with addition, in the antibody mix, of 2 µmol/L flavopiridol. For adipose tissue the enzymatic digestion was performed with 4 mg/mL collagenase II, resuspended in 0.25% BSA and 5 mM CaCl₂ and supplemented with 2 µ mol/L flavopiridol, for 30 min at room temperature under agitation. The rest of the procedure was performed as described above. 200 cells from each sample were directly sorted in a 96 well plate (Biorad) in 4 µL H₂O containing 0.2% Triton X-100 (Sigma) and 0.8 U/mL RNase inhibitor (Clontech). RNA was extracted with RNeasy mini kit (Qiagen), following manufacturer instructions and was quantified with ribogreen quantification (Thermo Scientific). Agilent bioAnalyzer was used for quality control. For each sample, 400pg of were amplified for 14 cycles with SMART-seq V4 (Clonetech) ultra-low input RNA kit for sequencing. Illumina HiSeq libraries were prepared with 10 ng amplified cDNA, using 8 cycles of PCR with Kapa library preparation chemistry kit (Kapa Biosystems). Barcoded samples were run on a HiSeq 2500 1T in a 50bp/50bp paired end run using TrueSeq SBS Kit V3 (Illumina). An average of 33 millions of read was generated per sample, with an average of 57% mRNA bases.

Fastq read files were aligned with Star Version 020201 to mm10 reference genome and quantified with Homer version 4.9, then subsequently analyzed in R with Bioconductor package Limma version 3.28.21 and Voom. For the cell-type analysis between groups differential gene expression was performed with FDR corrected P-value < 0.05 and absolute logFC > 1 of any group and the mean expression. K-means clustering was applied with 20 clusters, and highly correlating clusters with Spearman rank correlation higher than 0.9 were subsequently combined resulting in 8 clusters. Clusters were reordered according to number of genes. Functional enrichment was performed using Metascape. For the effects of food intake, linear model contrasts were set-up for each cell type for pairwise comparisons of the 10% lipid diet, 45% lipid diet, and 45%>10% lipid diet. Explained variance for all expressed genes in the RNA-seq data was calculated using the VariancePartition Bioconductor package.

qRT-PCR on Sorted Cells and Adipose Tissue Samples. For qRT-PCR, 10000 cells were sorted in 300 µL RNA lysis buffer (Macherey-Nagel, Nucleospin TriPrep), after preparation of the samples as described for RNA sequencing. RNA extraction was performed following manufacturer’s instructions (Macherey-Nagel, Nucleospin TriPrep), RNA concentration was measured with nanodrop2000. cDNA preparation was performed with Quantitect Reverse transcription kit (Qiagen) as per manufacturer’s instructions. qRT-PCR were done with 1 ng cDNA.

For qRT-PCR performed on eWAT and iWAT from Ccr2^{-/-} and littermate controls as well as Tnfrsf11a^{Cre}; Csf1r^{fl/fl} and littermates, tissue samples were weighed and snap frozen in liquid nitrogen. RNA extraction was performed on 20 mg of tissue following the same protocol as for the sorted cells and the qRT-PCR was performed on 10 ng cDNA. qRT- PCR are performed on a Quant Studio 6 Flex using TaqMan Fast Advance Mastermix, and TaqMan probes for ActinB (Mm02619580_g1), Gapdh (Mm99999915_g1), Tnf (Mm00443258_m1), Il1b (Mm00434228_m1), Il6 (Mm00446190_m1), leptin (Mm00434759_m1), 1110 (Mm01288386_m1), Adiponectin (Mm00456425_m1), Cxcl12 (Mm00445553_m1), Cxcl13 (Mm04214185_s1), Ctsl (Mm00515597_m1), Igf1 (Mm00439560_m1), Vegfb (Mm00442102_m1), Pdgfc (Mm00480205_m1), Bmp2 (Mm01340178_m1), Sparc (Mm00486332_m1), Sparcl1 (Mm00447784_m1), Timp1 (Mm01341361_m1), Timp2 (Mm00441825_m1).

Whole Mount Imaging and Cytology of Sorted Macrophages. For whole mount immunofluorescence imaging, after anesthesia and blood collection by cardiac puncture, the mice were perfused with 10 mL PBS at room temperature. Approximately 3 mm³ pieces of the tissue were incubated in 4% paraformaldehyde (PFA) (Electron Microscopy) diluted in PBS for 30 min at room temperature with agitation, then rinsed with PBS and stained with directly conjugated antibodies for 30 minutes. Then the samples were rinsed with PBS 3× and mounted on cavity slides (Sigma) with Fluoromount G (eBioscience). The antibodies used are listed in Tables 1 and 2. Approximately 50 µm thick Z-stack and tile scan were acquired with LSM880 Zeiss microscope with 40x/1.3 (oil). Image analysis was performed using Imaris (Bitplane) software.

For perilipin staining, samples were collected in 4% methanol free PFA, fixed for 2 days at 4° C., and embedded in paraffin. 5 µm slides were cut with a Leica RM2265 microtome, dewax with xylene and rehydrated with ethanol bath of decreasing concentrations. Antigen retrieval was performed with pH 6 citrate solution (Cell Signaling). After endogenous peroxidase blocking for 10 min with 3% H₂O₂ solution (Sigma), the samples were blocked with TBS (Sigma) Tween 0.3% (Sigma), BSA 5%, and Normal goat serum 5% (Sigma). Slides were incubated overnight at 4° C. with anti-mouse and human perilipin antibody (1/200, clone D1D8, Cell Signaling). Secondary antibody, goat anti rabbit HRP coupled antibody (1/200, cell signaling) was incubated for 1 hour at room temperature before revelation with peroxidase substrate kit (Vector, SK4805), and counterstaining with hematoxylin (0.25%, Sigma). Slides were finally dehydrated in ethanol bath of increasing concentration and xylene baths and mounted in Entellan (Merck). Paraffin sections were also stained with Hematoxylin and eosin. Images were taken with different objectives (N-Achroplan 2.5x/0.07, N-Achroplan 10x/0.25, N-Achroplan 20x/0.45, N-Achroplan 63x/0.85) of an Axio Lab.A1 Zeiss. Gross morphology of the liver and adipose tissues were taken with a Leica M80.

To quantify the size of the adipocytes, the software image J was used. Regions of interest (ROI) were drawn following the cellular membrane of the adipocytes and the surface of the ROI was determined by the software. 50 adipocytes were measured per field of view of the eWAT. Adipocytes were measured on 3 fields of view, taken with N-Achroplan 10x/0.25 objective. Crown-like structures (CLS) were quantified on the same fields of view. Crown like structures were defined as microscopic foci of dying adipocytes surrounded by macrophages as visualized by H&E staining.

Analysis of macrophages was done on May-Grunwald Giemsa staining of sorted cells from each population. 1000 to 2000 cells for each sample were sorted directly in FBS (Invitrogen), and centrifuged for 10 min at 800 g with low acceleration onto Superfrost slides (Thermo Scientific). After air drying for at least 30 min, the slides were fixed in methanol, air dried for at least 30 min, stained with May-Grunwald (Sigma) solution for 5 to 15 min then Giemsa (Sigma) solution 14% for 15 to 30 min, and rinsed with Sorenson buffer pH 6.8. After air drying, the slides were mounted with Entellan (Merck). Pictures were taken using Axio Lab.A1 Zeiss microscope with N-Achroplan 100x/01.25 objective.

In Vitro Epididymal Adipose Tissue Explants. Epididymal fat pads of 4-day-old Tnfrsf11a^{Cre+}Csf1r^{fl/fl} mice and littermates were dissected and incubated in RPMI with 10% heat de-activated FBS at 37° C. for 14 days. Fresh medium was added to the explants on day 7. At the termination of the experiment, the explants were fixed in 4% PFA for 30 minutes at room temperature with agitation and then rinsed in PBS and permeabilized for 15 minutes in PBS + 0.03% Triton. Subsequently, the explants were blocked with 2% BSA and stained with directly conjugated antibodies. To remove macrophages from wt epididymal fat pads in vitro, 15 µM of the CSF1R inhibitor PLX5622 was added to explants on day 0 and 7 following the addition of fresh medium. For the in vitro rescue experiments, epididymal fat pads of Tnfrsf11a^{Cre}; Csf1r^{fl/fl} mice and littermates as well as PLX5266 treated fat pads were supplemented with 100 ng/ml of recombinant mouse PDGFcc (R&D systems), 10 ng/ml of recombinant mouse VEGFb (R&D systems), or 10 ng/ml of recombinant mouse IGF1 (R&D systems) three times on days 0, 3, and 6.

Drosophila Lines and Crosses. Flies were raised at 25° C. in a 12-h light/12-h dark cycle and maintained on food containing 10% w/v Brewer’s yeast, 8% fructose, 2% polenta and 0.8% Agar. Adult flies in vials were allowed to lay eggs for 2 hours, whereupon the adults were removed, and eggs were allowed to develop until the wandering larval stage. Table 3 shows a list of all of the Drosophila lines used herein.

TABLE 3

Drosophila Lines Used in the Experiments Described Herein

| | |
|---|---|
| Fly stock | Description |
| w¹¹¹⁸; Srp^{Hemo}Gal4 | Plasmatocyte specific line. A kind gift from Norbert Perrimon |
| w¹¹¹⁸;; uas-reaper | A pro-apoptotic gene. BDSC # 50790 |
| w¹¹¹⁸;He-gal4 | BDSC #8699 |
| w¹¹¹⁸;uas-pvf3 | A kind gift from Michael Galko |
| w¹¹¹⁸;uas-dilp5-IR | VDRC # 105004 |
| w¹¹¹⁸;uas-dpp-IR | BDSC #25782 |
| w¹¹¹⁸;uas-pvf1-IR | BDSC #39038 |
| w¹¹¹⁸;uas-pvf3-IR | BDSC #38962 |
| y¹ w¹¹¹⁸ | BDSC #1495 |
| y¹ w¹¹¹⁸;Pvf3^{EY09531} | BDSC #17577 |

Triglyceride measurement, buoyancy assay, fat body cell size, and developmental timing in Drosophila. To measure triglycerides, 10 wandering L3 larvae were homogenized in 100 µl of PBS + 0.05% Tween 20 with a pestle on ice. Triglyceride was then measured using the free glycerol reagent (Sigma; F6428) as described before and normalized to the protein content. In a complementary experiment, the lipid content of the wandering L3 larvae were compared using buoyancy assay. The L3 larvae were placed in a 30% sucrose solution and then diluted slowly to an 8% sucrose solution with PBS. Afterwards, the larvae were left to equilibrate for 30 minutes at which point the position of the larvae in the solution was recorded.

To measure the size of the fat body cells, the larvae fat bodies were dissected out and then fixed in 4% PFA for 30 minutes at room temperature. The fixed tissues were then stained with Bodipy for 30 minutes and washed and imaged on a Zeiss LSM 880 with a 40X objective.

To examine the development of Drosophila larva, 30 eggs were seeded on freshly prepared food and then left to develop to the L3 stage.

qPCR on Sorted Drosophila Hemocytes and Whole Larvae. For qRT-PCR, 10000 hemocytes were sorted into 300 µL of Trizol LS (Life Technologies). RNA extraction was performed following manufacturer’s instructions (Direct-zol RNA microPrep, Zymo Research). RNA concentration was measured with nanodrop2000. cDNA preparation was performed with Quantitect Reverse transcription kit (Qiagen) as per manufacturer’s instructions. qRT-PCR were done with 1 ng cDNA.

For qRT-PCR performed on whole larvae, 5-10 larvae were homogenized in Trizol with a pestle on ice. RNA extraction and cDNA preparation was as described above. The same protocol as for the sorted cells and the qRT-PCR was performed on 10 ng cDNA. qRT-PCR are performed on a Quant Studio 6 Flex using TaqMan Fast Advance Mastermix, and TaqMan probes for gapdh2 (Dm01843776_s1), dilp2 (Dm01822534_g1), dilp4 (Dm01801938_g1), dilp5 (Dm01798339_g1), dilp6 (Dm01829746_g1), pvf1 (Dm01813949_m1), pvf3 (Dm01814376_m1), and dpp (Dm01842959_m1).

Recombinant mouse PDGFcc was purchased from R&D systems (Cat# 1447-PC-025/CF). It is the active/cleaved form of PDGFcc that extends from Va1235-G1y345. The protein has the following sequence (SEQ ID NO: 3):

VVNLNLLKEEVKLYSCTPRNFSVSIREELKRTDTIFWPGCLLVKRCGGNCACCLHNCNECQCVPRKVTKKYHEVLQLRPKTGVKGLHKSLTDVALEHHEECDCVCRGNAGG

Statistical Tests. Data are represented as individual values per mouse, unless otherwise stated. The n value represents biological replicates. Statistical significance was calculated using the software GraphPad Prism using unpaired Student’s t-tests to compare 2 groups or one-way ANOVA, to compare more than 2 groups, as indicated in the figures. For RNA sequencing, R software was used, as described above.

### Example 2: White Adipose Tissue Deficiency in Mice Lacking Csflr

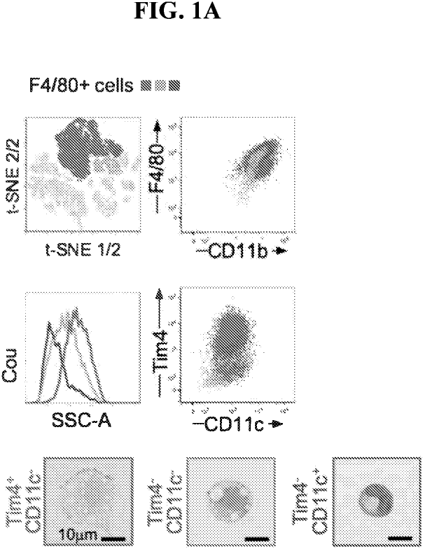

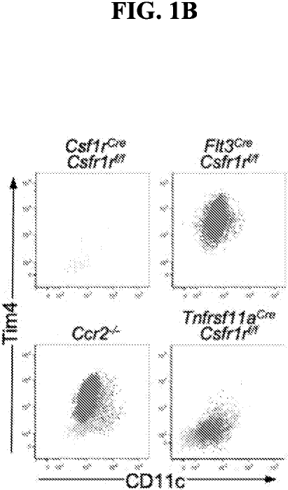

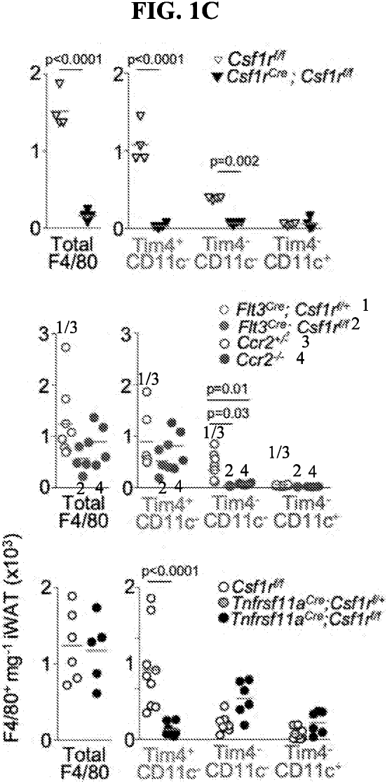

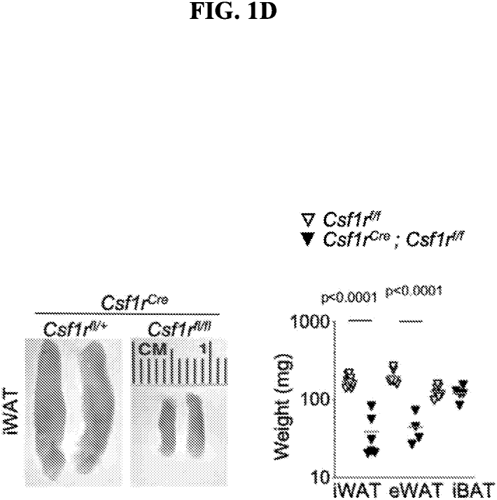

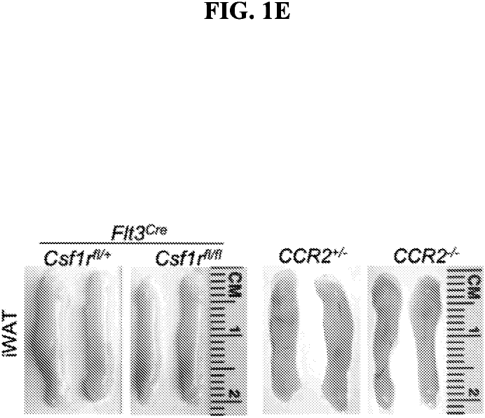

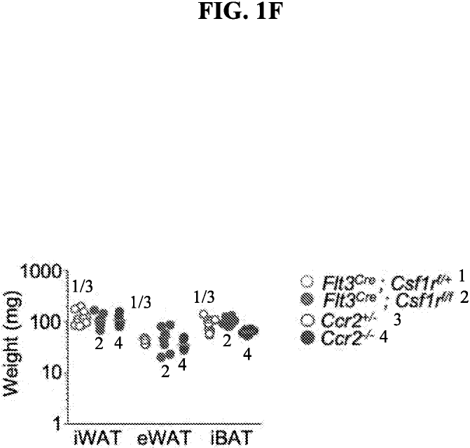

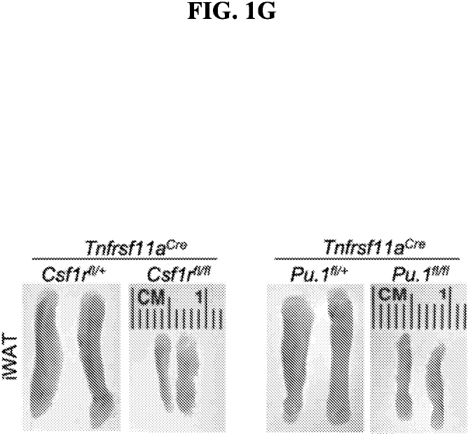

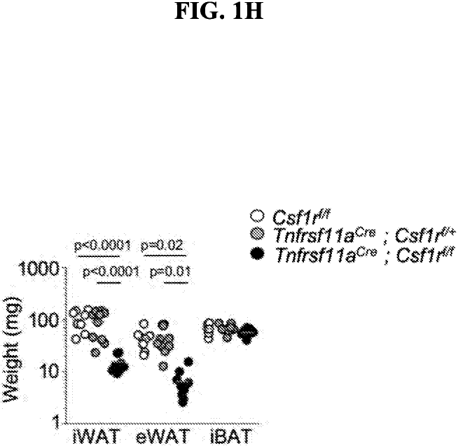

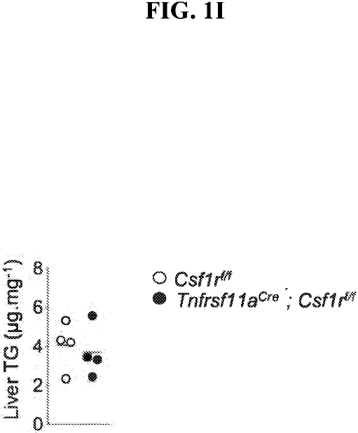

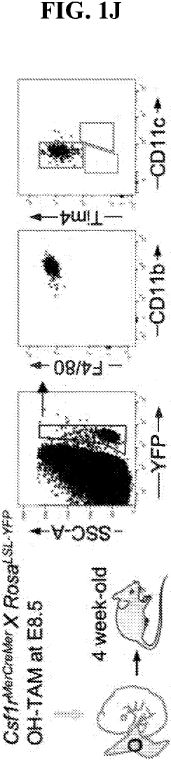

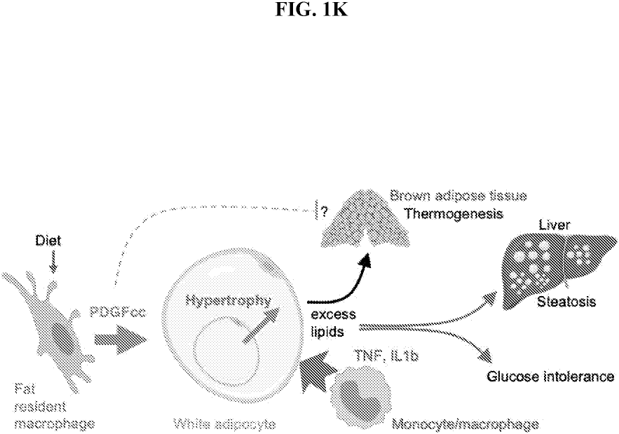

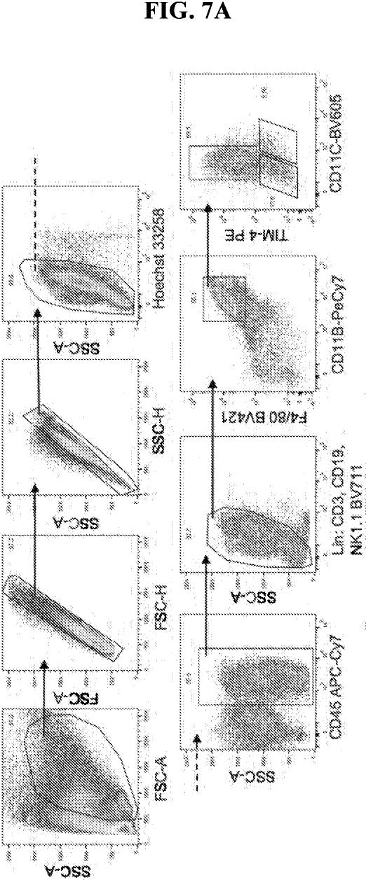

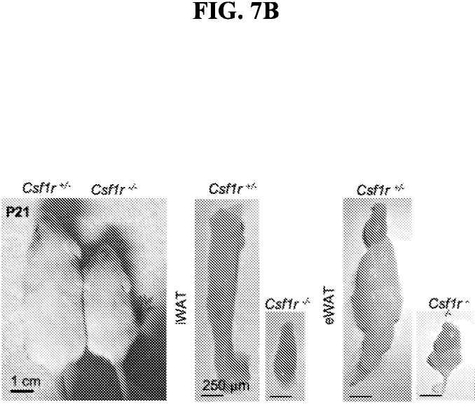

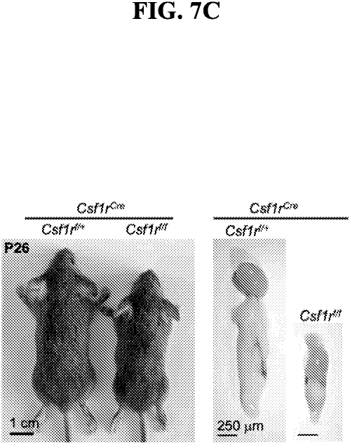

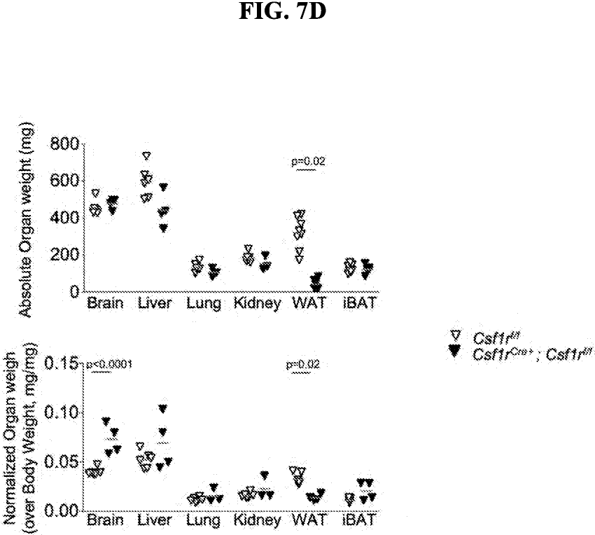

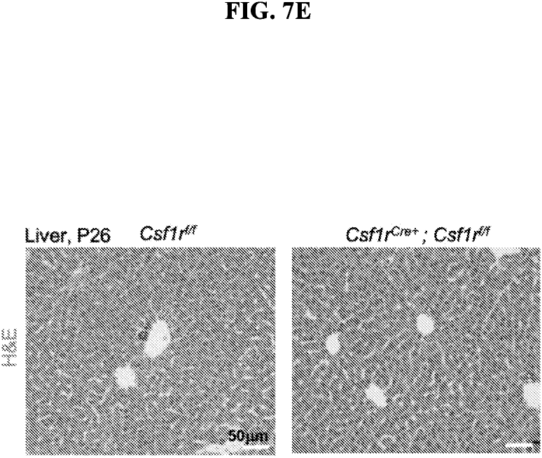

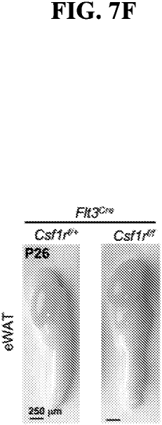

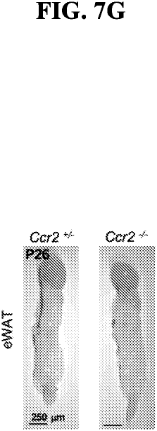

Mice carrying a homozygous deletion of the Csf1 receptor (Csf1r^{-/-} and Csf1r^{Cre}; Csf1r^{f/f}) lack macrophages in most tissues, including the fat-pads (FIGS. 1A-1C, FIG. 7A), and present with skeletal deformities (osteopetrosis) attributed to lack of osteoclasts and abnormal brain development attributed to lack of microglia. It was also observed that both lines of Csf1r-deficient mice presented with a 90% reduction in size and weight of visceral and sub-cutaneous white adipose tissues (WAT) at one month of life (FIG. 1D, FIGS. 7B-7C). Adipose tissue deficiency is also reported in Csf1r deficient rats and macrophage-deficient Trib1 knockout mice. The weight of brain, lung, kidney and interscapular brown adipose tissue were not different between Csf1r-deficient and control littermates (FIG. 7D). The liver was not enlarged and did not show steatosis (FIG. 7E). These results suggest an adipose tissue deficiency in Csf1r-deficient mice.

### Example 3: Tissue Resident Macrophage Deficiency Accounts for Fat Tissue Defect

Csf1r deletion in the Flt3-expressing hematopoietic stem cell lineage, and Ccr2 deficiency, in which myeloid cell egress from the bone marrow and entry into WAT is minimal, both resulted in the loss of small Tim4⁻F4/80⁺ cells in adipose tissue while large Tim4⁺F4/80⁺ cells were still present (FIGS. 1A-1C), and white adipose tissue mass and histology were normal (FIGS. 1E-1F, FIGS. 7F-7G). Therefore, a deficiency in HSC-derived monocyte/macrophage is unlikely to account for the adipose tissue defect in Csf1r deficient mice.

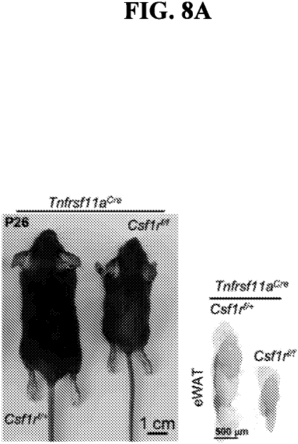

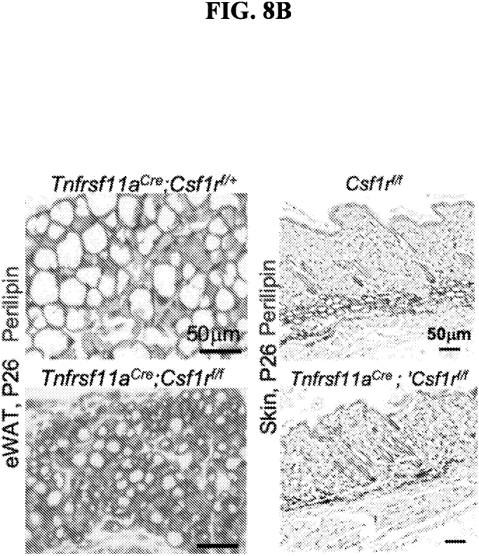

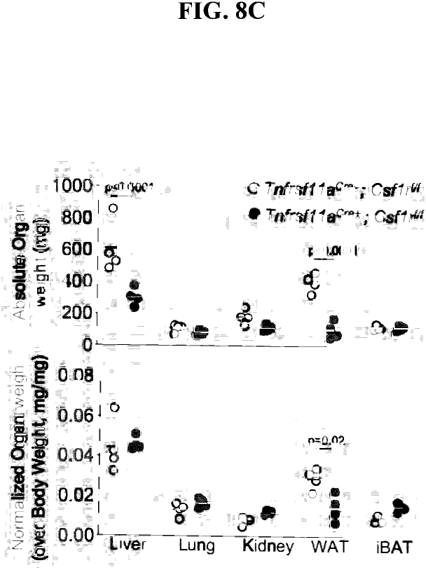

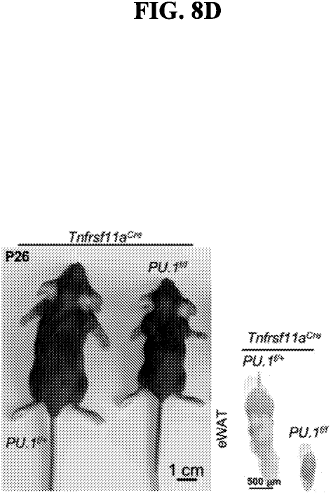

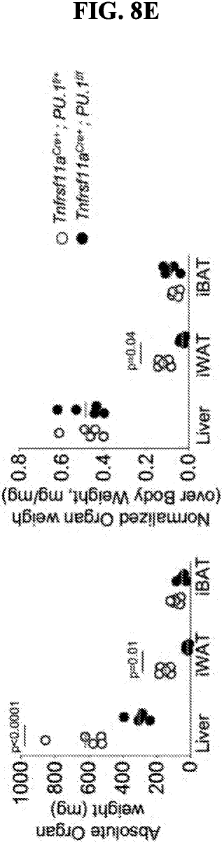

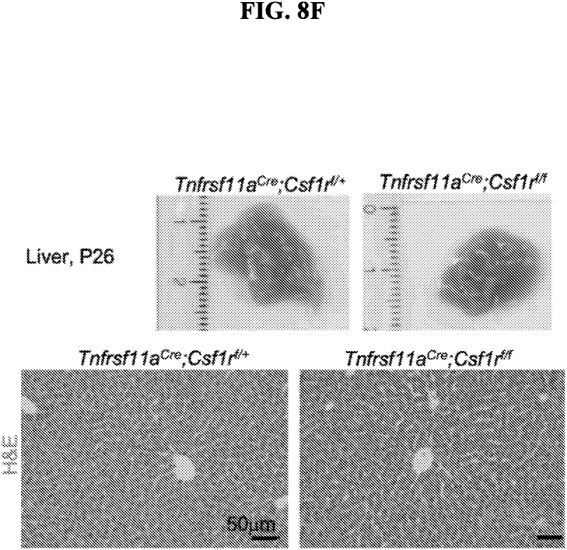

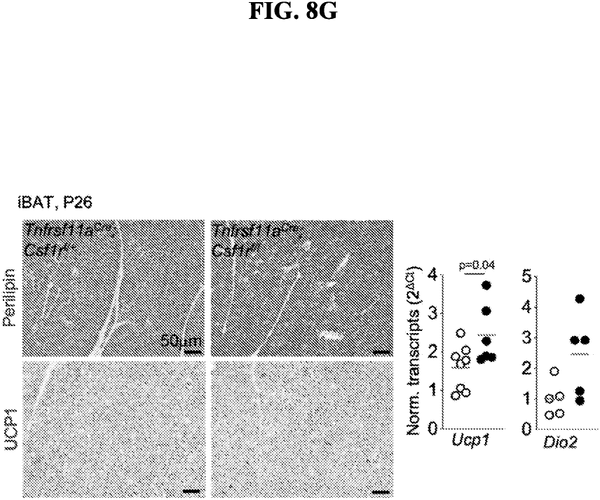

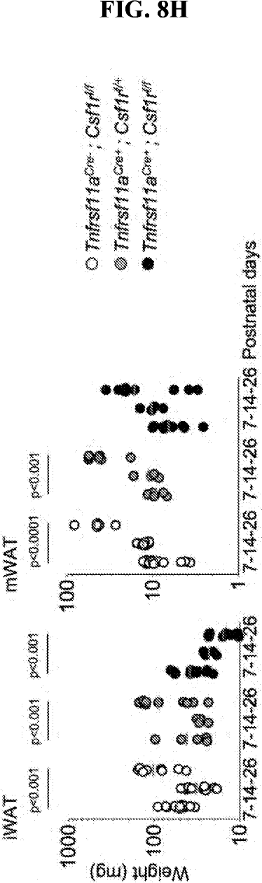

In contrast, Tnfrsf11a^{Cre}; Csf1r^{f/f} mice, as well as Tnfrsf11a^{Cre}; PU.1^{f/f} mice, which both lack embryonic erythro-myeloid progenitors (EMP)-derived resident macrophages, and present with self-resolving osteopetrosis, lacked large Tim4⁺F4/80⁺ cells (FIGS. 1A-1C) and recapitulate the adipose tissue phenotype of Csf1r^{-/-} mice with a selective ~90% decrease in the weight of visceral and sub-cutaneous white adipose tissue in comparison to littermate controls (FIGS. 1G-1H, FIGS. 8A-8E). Liver triacylglycerols were not increased in mutant Tnfrsf11a^{Cre}; Csf1r^{f/f} mice (FIG. 1I). Liver histology was no different from control, and the liver was not enlarged, in fact its absolute weight was slightly reduced (FIGS. 8C-8F). Interscapular brown adipose tissue (iBAT) morphology and weight were similar to control, but Ucpl expression was elevated in mutant mice (FIG. 1H, FIGS. 8C, 8E, 8G). Thus, mice lacking resident macrophages present with reduced fat mass similar to Csf1r-deficient mice without exhibiting any detectable signs of lipodystrophy.

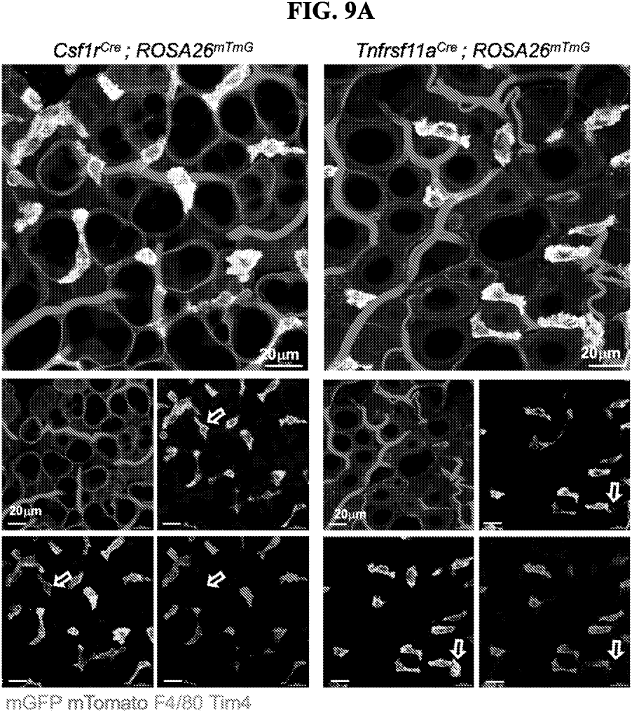

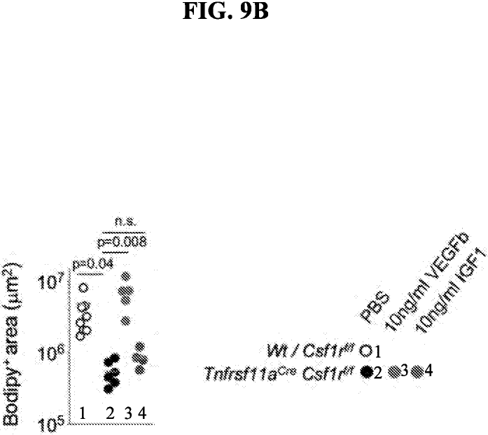

A lineage mapping analysis of E8.5 EMPs, confirmed labeling of large adipose tissue F4/80⁺ Tim4⁺ macrophages, but not of small F4/80⁺ Tim4⁻ cells (FIG. 1J). In situ lineage-tracing analysis of Csf1r- and Tnfrsf11a- expressing cells in Csf1r^{Cre}; Rosa26^{mT/mG} and Tnfrsf11a^{Cre}; Rosa26^{mT/mG} mice showed GFP expression by F4/80⁺ macrophages that surround adipocytes, while all other cells expressed membrane-bound tdTomato, indicating that Cre-recombinase activity only occurs in F4/80⁺ macrophages or their progenitors (FIG. 9A). Additionally, GFP expression was restricted to Tim4+ F4/80⁺ macrophages in Tnfrsf11a^{Cre}; Rosa26^{mT/mG} mice, confirming the specific targeting of Tim4+ resident macrophages in the fat pads of Tnfrsf11a^{Cre}; Csf1r^{f/f} mice (FIG. 9A). These data altogether suggest that the adipose tissue deficiency in Csf1r-deficient mice may be attributable to resident macrophage deficiency, while HSC-derived Ccr2-dependent Tim4⁻ macrophages appear to be neither required nor sufficient for white adipose tissue development in mice.

### Example 4: White Adipocytes From Mice Lacking Resident Macrophages Fail to Store Lipids

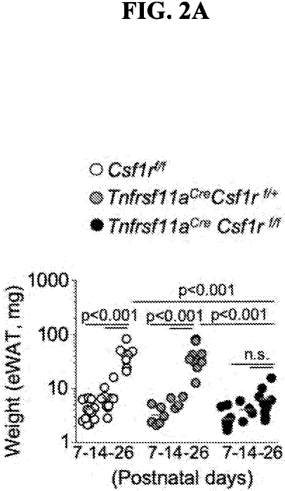

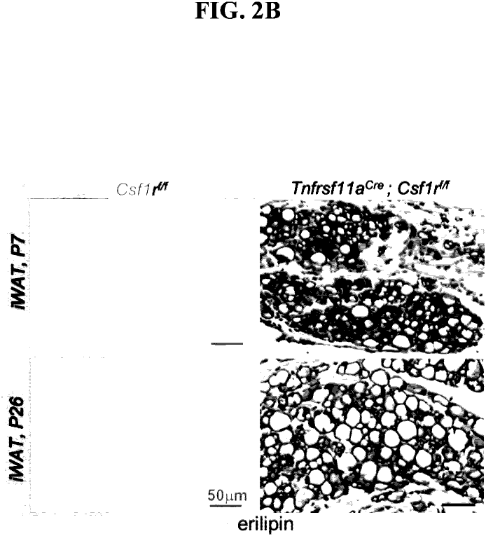

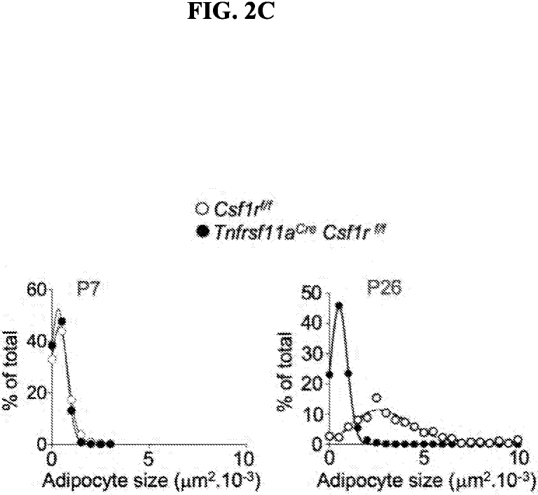

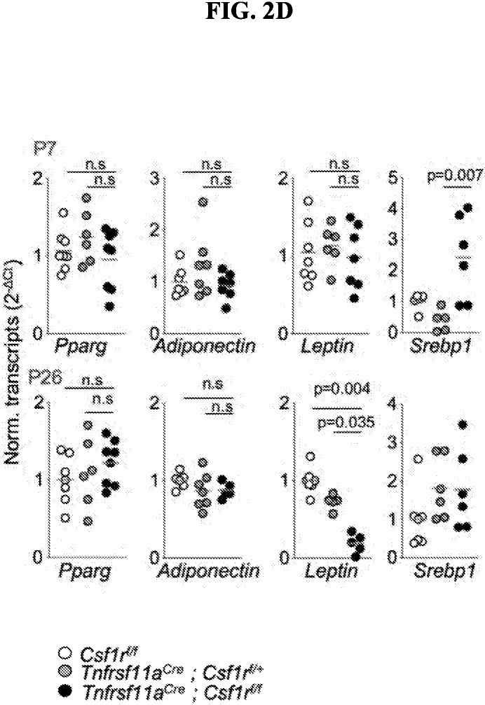

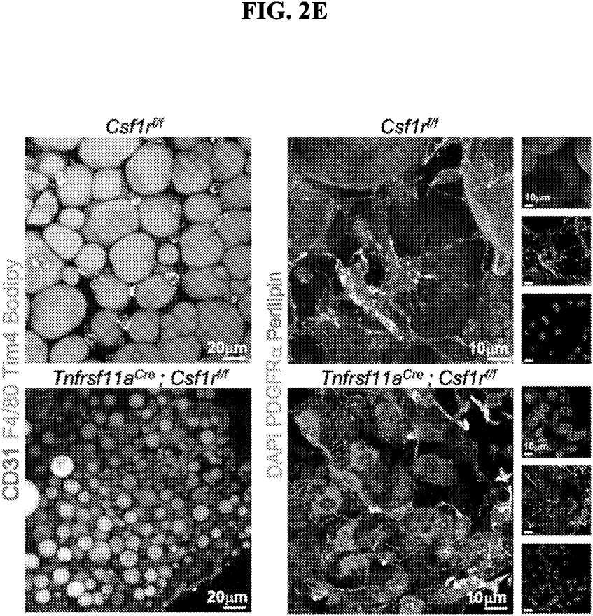

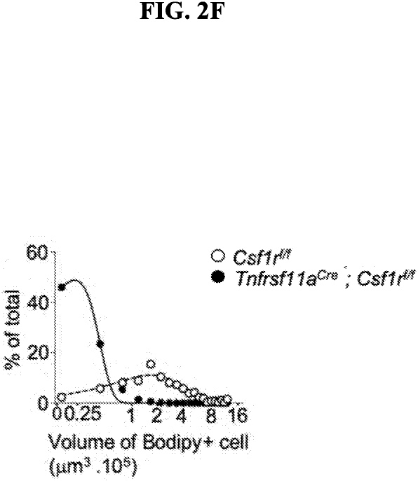

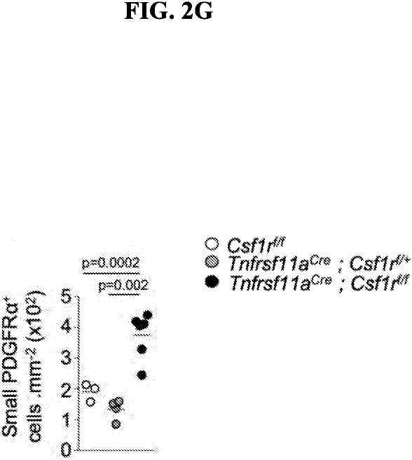

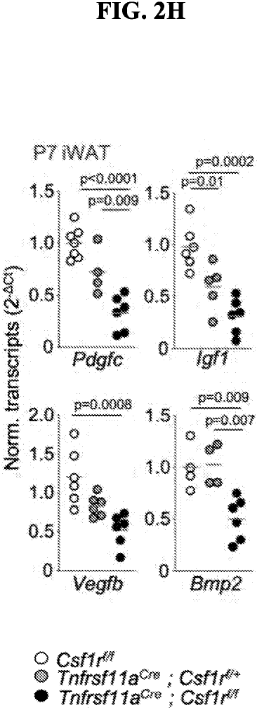

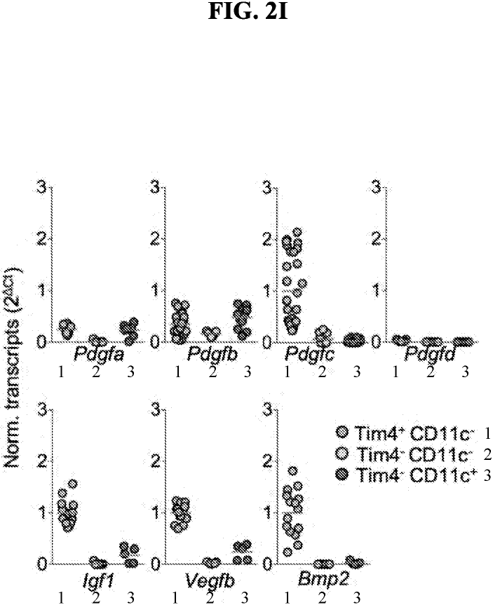

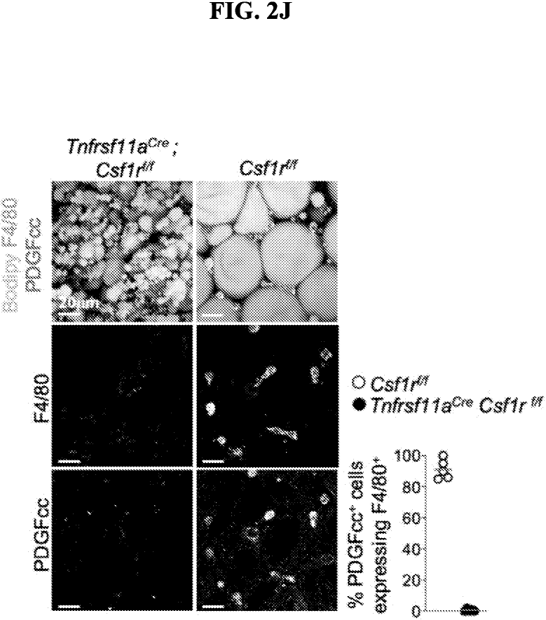

Next, the adipose tissue deficiency in Tnfrsf11a^{Cre}; Csf1r^{f/f} mice was characterized. A time-course analysis indicated that neonatal (P7) fat pads from Tnfrsf11a^{Cre}; Csf1r^{f/f} mutant mice and control littermates had similar weights (FIG. 2A, FIG. 8H) and both contained small perilipin⁺ Bodipy+ cells (FIG. 2B). However, mutant fat pads failed to grow during the first month of life as perilipin⁺ cells remained small in the mutant inguinal, epidydimal, and mesenteric fat pads (FIGS. 2A-2C, FIG. 8H). A qPCR analysis indicated that adipocyte genes Pparg, Adiponectin, Leptin, and Srebpl were expressed at P7 and at 26 in mutant fat-pads, albeit Leptin expression was decreased at P26 consistent with decreased lipid mass (FIG. 2D). Whole mount confocal analysis confirmed that bodipy⁺ perilipin⁺ adipocytes were present in one month old Tnfrsf11a^{Cre};Csf1r^{f/f} fat pads, with ~10-fold reduction in bodipy⁺ lipid content (FIGS. 2E-2F), which may account for the 90% decrease in the fat pads weight (FIG. 1H). Whole mount confocal analysis also confirmed that Tim4⁺ macrophages were absent from fat pads from Tnfrsf11a^{Cre};Csf1r^{f/f} mice in comparison to control littermates (FIG. 2E), but CD31⁺ capillaries as well as PDGFRα⁺ stromal cells were present (FIGS. 2E-2G). Therefore the 90% reduction in adipose tissue observed in Tnfrsf11a^{Cre};Csf1r^{f/f} mice may be accounted for by the specific lack of resident macrophages and decreased storage of bodipy⁺ lipids in perilipin⁺ adipocytes, although these experiments do not eliminate the possible contribution of reduced proliferation/differentiation at the level of pre-adipocytes or perilipin-expressing cells.

### Example 5: PDGFcc Deficiency in Fat Pads From Mice Lacking Resident Macrophages

A lipid storage defect can reflect decreased nutrient intake or increased expenditure, as well as changes in growth factors that directly or indirectly control adipocyte lipid storage. A qPCR analysis of inguinal fat pads indicated that expression of Pdgfc and Igf1 were strongly reduced in fat pads from mutant mice as compared with control littermates (FIG. 2H). Expression of Vegfb and Bmp2 was also reduced but to a lesser extent (FIG. 2H). A qPCR analysis of FACS-sorted adipose tissue macrophages indicated that wild-type Tim4⁺ macrophages expressed the pro-adipogenic growth factors Pdgfc, Vegfb, Bmp2 and Igf1 (FIG. 2I). Immunostaining with PDGFcc antibodies demonstrated that ~90% of PDGFcc expressing cells were macrophages in wt iWAT, and that PDGFcc expression is not detectable in mutant fat pads that lacked Tim4⁺ macrophages (FIG. 2J), indicating that macrophages represent the main local source of PDGFcc in white adipose tissue. Altogether, the above data suggest that reduced white adipose tissue in Tnfrsf11a^{Cre}; Csf1r^{f/f} mice results from decreased lipid storage in adipocytes, associated with the lack of Tim4⁺ resident macrophages and the adipogenic growth factors they normally produce. A possible role of PDGFcc is interesting as PDGF receptors alpha and beta have pleiotropic roles, including control of adipocyte differentiation and size. However, the interpretation of these data remains complicated by the pleotropic phenotype of the macrophage-less mutants mice.

### Example 6: Macrophages Control Lipid Storage in Drosophila Fat Body via PDGF Signaling

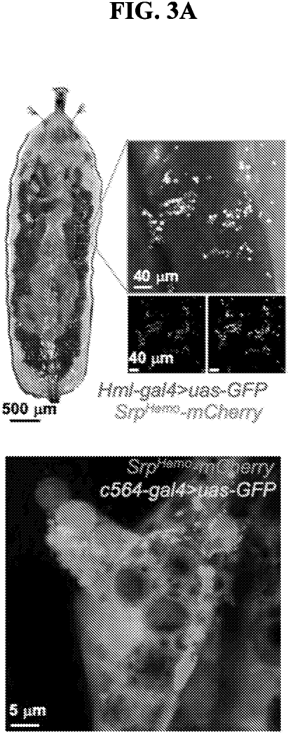

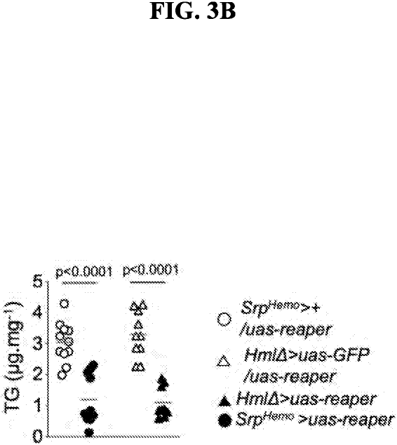

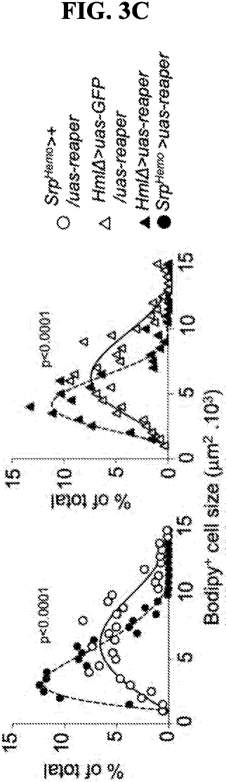

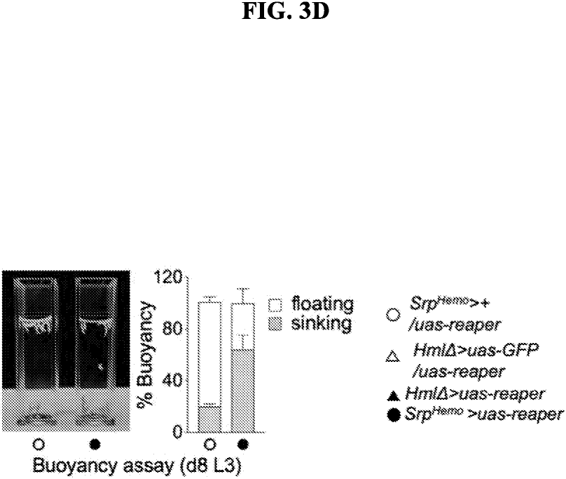

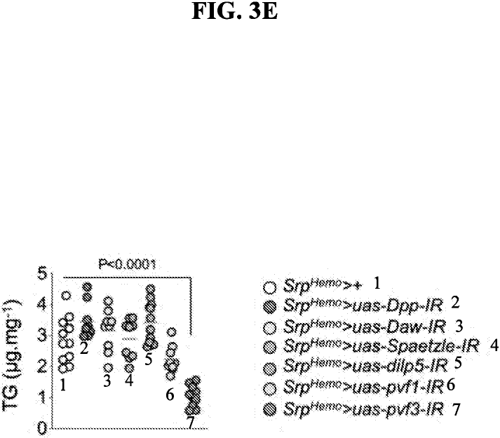

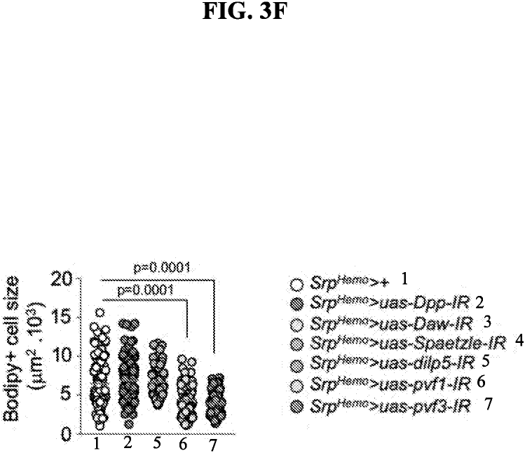

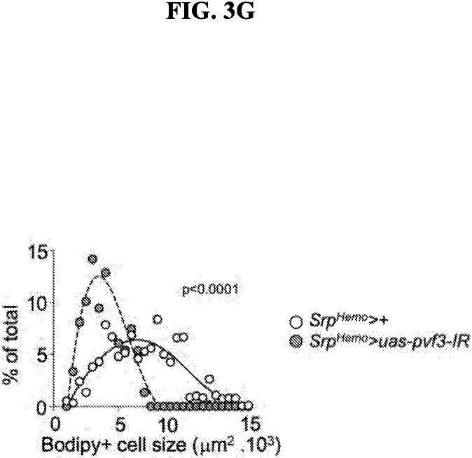

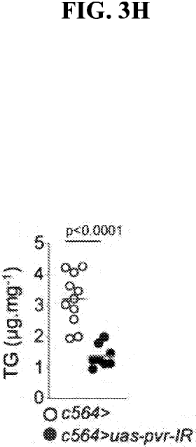

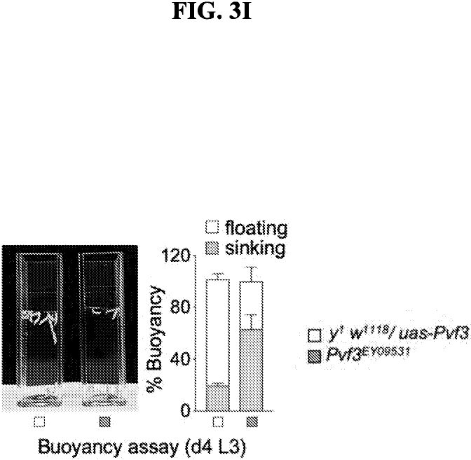

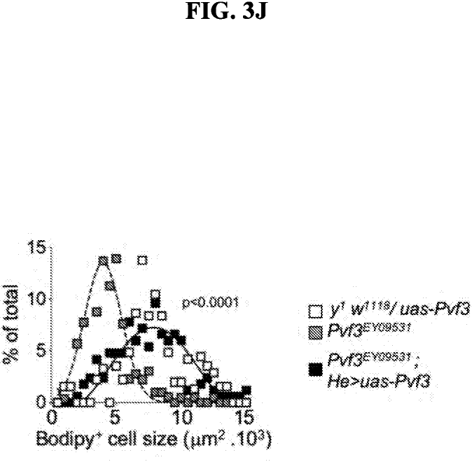

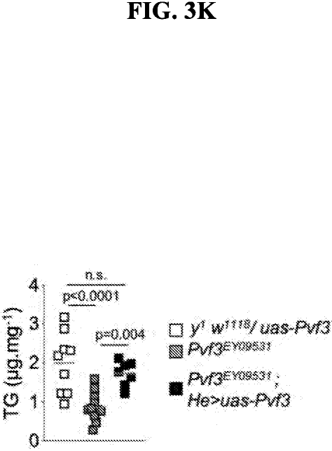

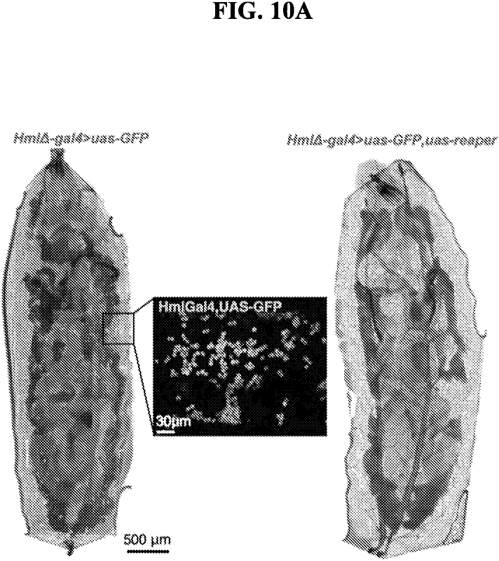

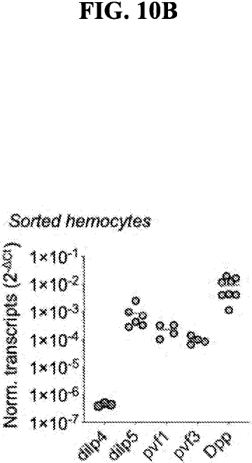

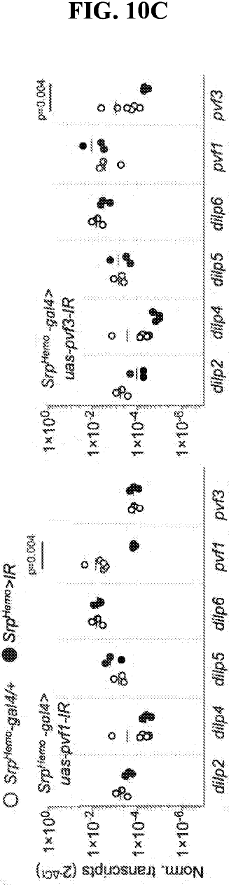

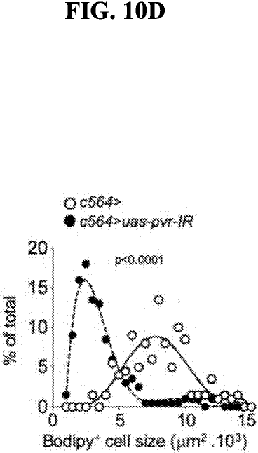

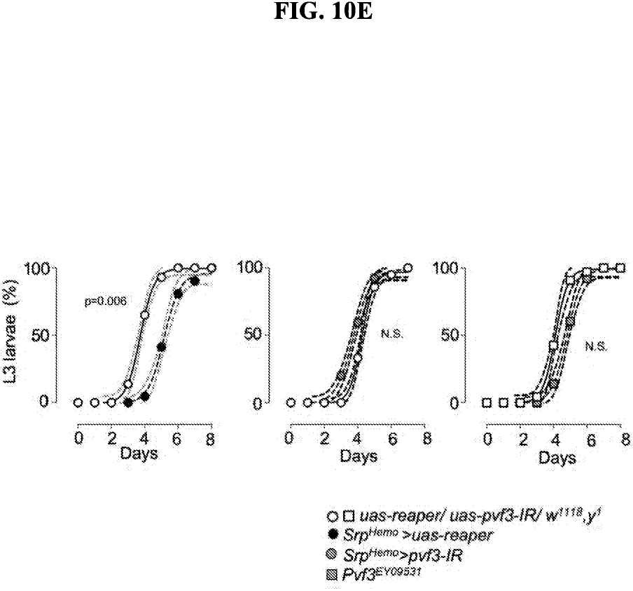

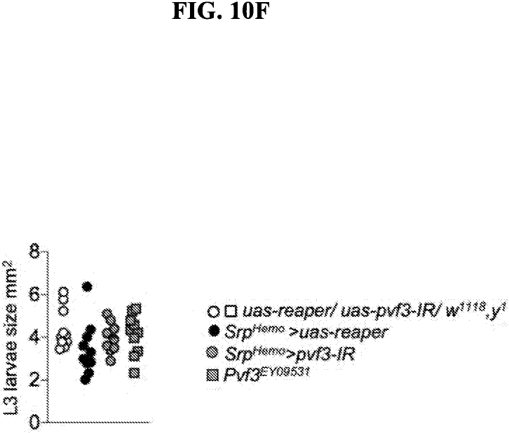

To directly test the roles of macrophage-derived growth factors in lipid storage, Drosophila was used as a genetically tractable metazoan model. Professional fat storing cells, macrophages, and the PDGF/VEGF family of growth factors are all conserved across the animal kingdom, including in Drosophila. Genetic labeling of Drosophila macrophages, hemocytes, with 2 independent reporters Hml-gal4>uas-GFP and Srp^{Hemo}-mCherry confirmed a close association between hemocytes and the dedicated fat storing tissue (fat body), labelled with c564-gal4>uas-GFP⁺ in L3 larvae (FIG. 3A). Hemocyte-deficient Drosophila were generated by inducing apoptosis in hemocytes using Srp^{Hemo}-gal4>uas-reaper or Hml-gal4 >uas-reaper lines. (FIGS. 3B-3C, FIG. 10A). In both models, a ~60% reduction in triglyceride content and fat body cell size was observed as compared to control larvae (FIGS. 3B-3C). This decrease was associated with reduced buoyancy in hemocyte-deficient Srp^{Hemo}-gal4>uas-reaper wandering L3 larvae (FIG. 3D). Next, whether growth factors produced by Drosophila hemocytes (FIG. 10B) are important for storage of triglycerides by fat body cells was investigated. Hemocyte-specific RNAi of Bmp (Bmp, Daw), Igf (dilp5), and Il1 (Spätzle) orthologs resulted in L3 larvae with normal triglyceride content and fat body cell size (FIGS. 3E-3F). In contrast, hemocyte-specific RNAi of Pvf3 (Srp^{Hemo}-gal4>uas-pvf3-IR) a PDGF/VEGF family ortholog resulted in larvae with a ~60% decrease in triglyceride content and fat body cell size (FIGS. 3E-3G, FIG. 10C). Larvae from hemocyte-specific Pvf1 RNAi presented with a milder phenotype: smaller fat body cells but no significant reduction in triglycerides (FIGS. 3E-3F, FIG. 10C). PDGF/VEGF family orthologs Pvf3 and Pvf1 share the receptor Pvr, expressed by fat body cells. As expected, RNAi of Pvr in the fat body, c564-gal4>uas-pvr-IR, yielded L3 larvae with reduced triglyceride content and smaller fat body cells similar to hemocyte-deficient and Pvf3 RNAi larvae (FIG. 3H, FIG. 10D).

To control for possible off-target effects of the RNAi constructs, Pvf3 mutant flies (Pvf3^{EY09531}) were analyzed. L3 Pvf3^{EY09531} larvae had reduced buoyancy and a ~70% reduction in triglyceride content and fat body cell size (FIGS. 3I-3K). Of note, hemocyte-deficient L3 larvae presented with a ~1 day developmental delay, but Pvf3^{EY09531} and Srp^{Hemo}-gal4>uas-pvf3-IRlarvae develop in time and with normal size (FIGS. 10E-10F).

Finally, it was verified that genetic rescue of Pvf3 expression in Pvf3^{EY09531} larvae also restored triglyceride content and fat body cell size (Pvf3^{EY09531}; He-gal4 ⁸²>uas-pvf3) (FIG. 3K). These data strongly suggest that Drosophila hemocytes control triglyceride storage in fat body cells via the production of PDGF/VEGF-family ortholog Pvf3 by hemocytes acting directly on Pvr expressing fat body cells, and suggest an evolutionarily conserved role for growth factor production by macrophages in control of lipid storage by fat storing cells.

### Example 7: PDGFcc Locally Produced by Resident Macrophages is Required and Sufficient for Lipid Storage by Adipocytes in the Mouse Fat-Pad

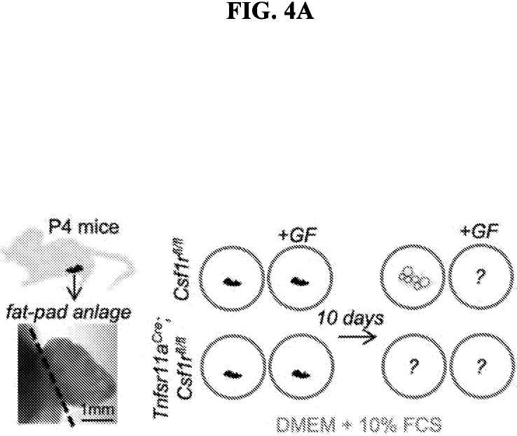

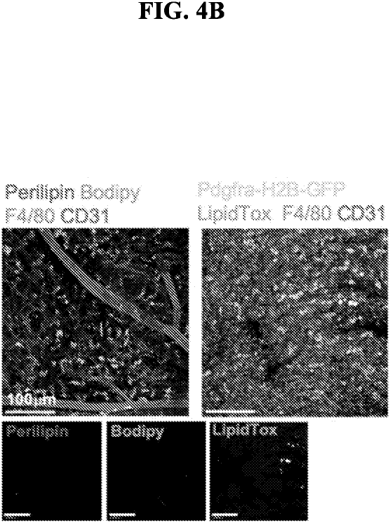

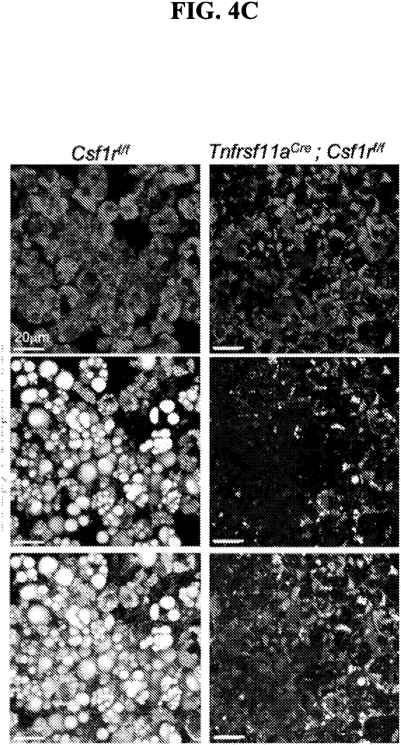

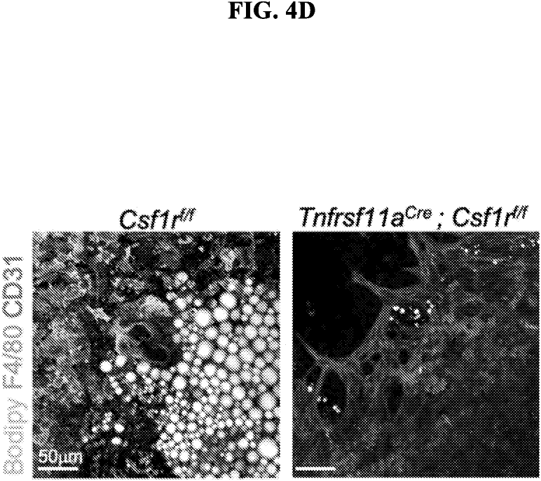

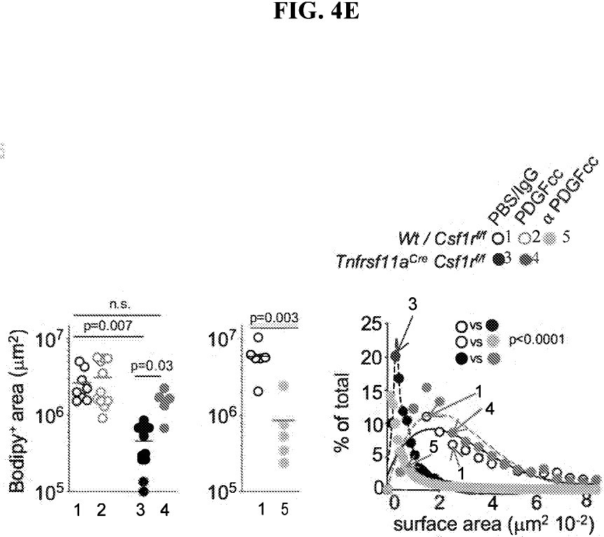

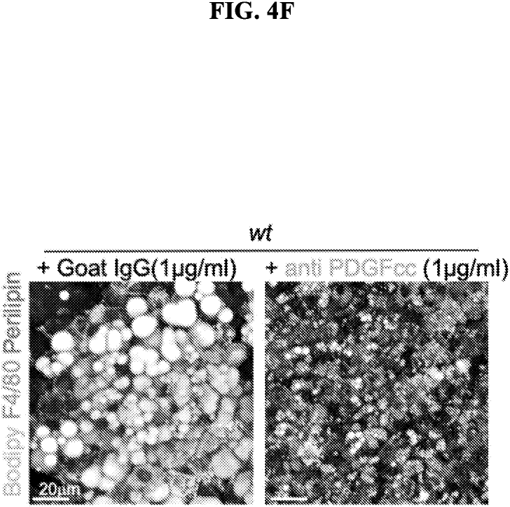

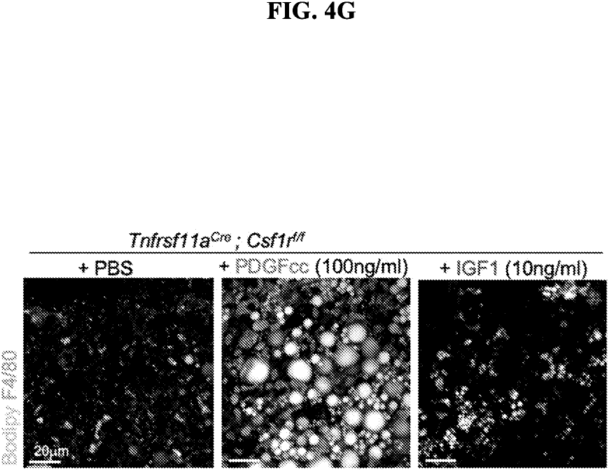

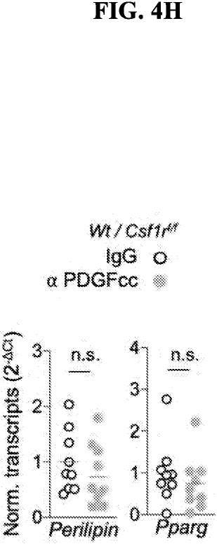

To address the critical question of whether the role of macrophages and PDGF-family growth factors on lipid storage can be observed in murine fat-pads, independently of the systemic and pleotropic effects of macrophage depletion, loss-of-function and rescue experiments were performed in murine isolated fat-pad explants (FIG. 4A). Wild type murine neonatal (P4) epidydimal fat pad anlagen contained PDGFRα⁺ cells, capillaries and macrophages, but lacked perilipin⁺, bodipy⁺ or LipidTox⁺ cells (FIG. 4B). As previously shown, perilipin⁺ bodipy⁺ adipocytes develop ex vivo in 10 days from wild-type explant cultures with conventional non-adipogenic medium (FIGS. 4C-4E). Perilipin⁺ cells also developed in fat pads explants from Tnfrsf11a^{Cre}; Csf1r^{f/f} mice, but they contained little to no lipids as evidenced by scant bodipy staining as compared to fat-pads from control littermates (FIGS. 4C-4E). Treatment of wild-type fat pads with anti-PDGFcc neutralizing antibodies (AF1447) phenocopy Tnfrsf11a^{Cre}; Csf1r^{f/f} fat-pad explants, with the development of small perilipin⁺ bodipy^{low/neg} cells (FIGS. 4E-4F). Symmetrically, treatment of Tnfrsf11a^{Cre}; Csf1r^{f/f}fat pads with recombinant PDGFcc -but not IGF1- rescues bodipy staining to control levels (FIGS. 4E-4G, FIG. 9B). To investigate whether PDGFcc modulates adipocyte differentiation or metabolism in fat pad explants, expression of Perilipin, and Pparg in wild type fat pads treated with goat IgG or anti-PDGFcc neutralizing antibodies was compared. Expression of Perilipin and Pparg were unchanged in fat-pads treated with anti-PDGFcc (FIG. 4H)).. These data indicate that, in an ex-vivo system, macrophages and PDGFcc are required to support lipid storage by adipocytes in a white adipose tissue autonomous manner. PDGFcc supplementation was also sufficient to rescue lipid storage by adipocytes in the absence of macrophages. Of note, it was observed that in vitro treatment with VEGFb can also rescue lipid storage (FIG. 9B), suggesting that several macrophage growth factors may act directly and/or indirectly, e.g., via endothelial cells, to support lipid storage. Accordingly, these results demonstrate that PDGFcc antagonists are useful in methods for treating or preventing lipid accumulation in adipose tissue in a subject in need thereof. In addition, these results also demonstrate that PDGFcc or PDGFcc agonists are useful in methods for treating lipodystrophy in a subject in need thereof.

### Example 8: PDGFcc Blockade Prevents Adipocyte Hypertrophy and Promotes Thermogenesis in Adult Mice Fed a Lipid-Rich Diet

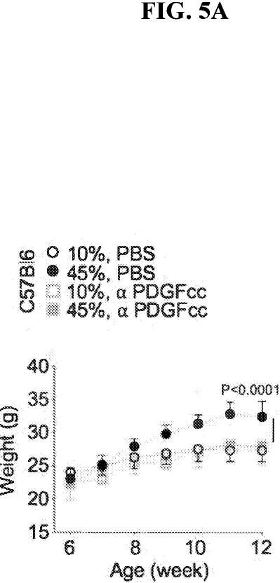

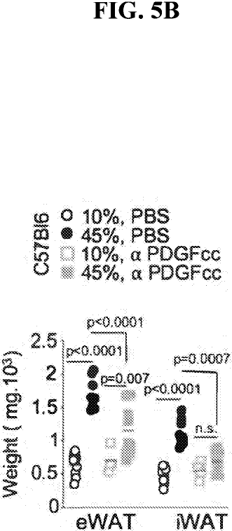

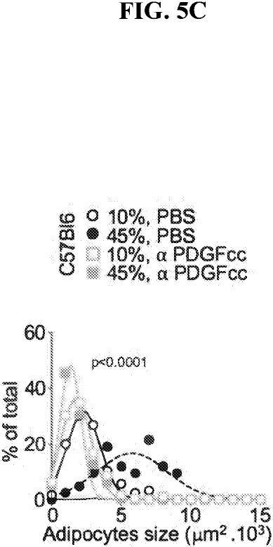

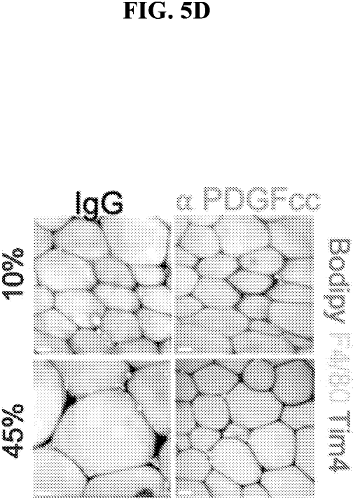

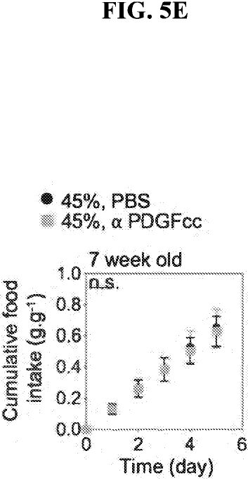

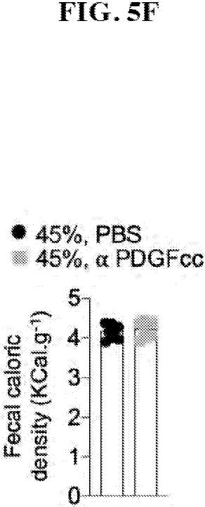

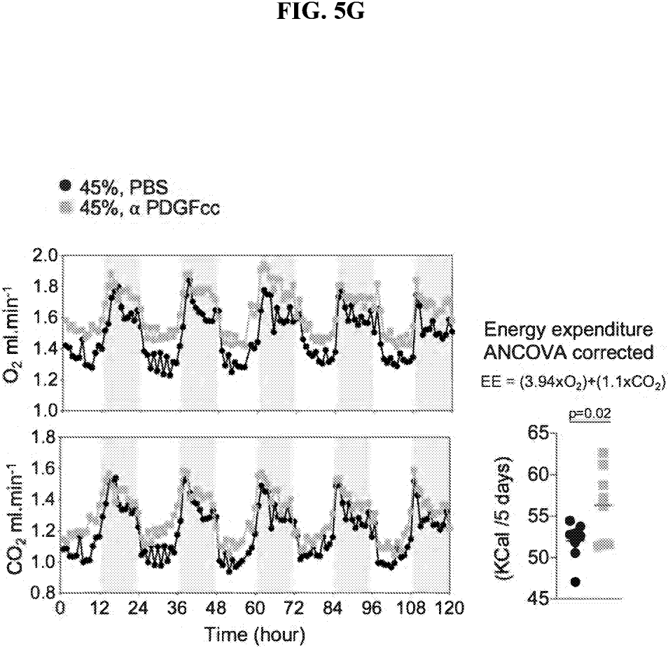

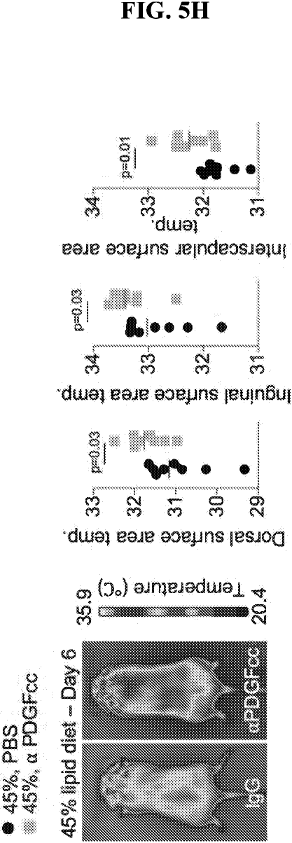

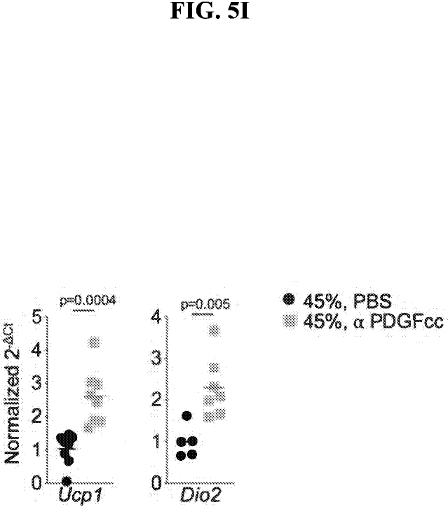

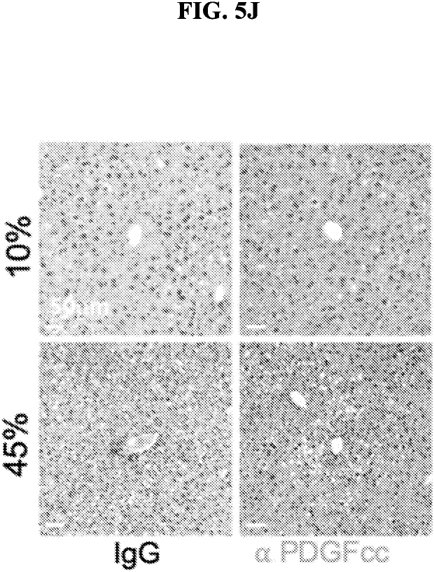

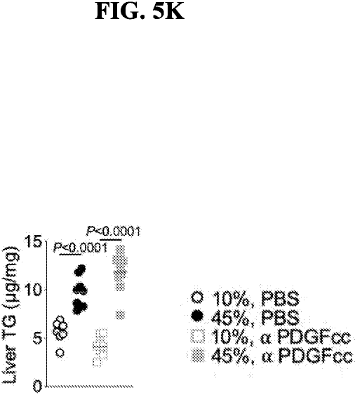

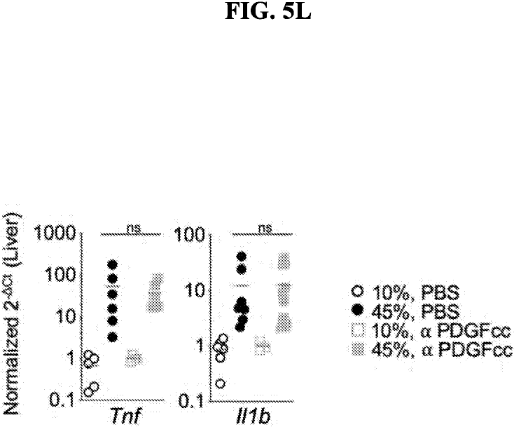

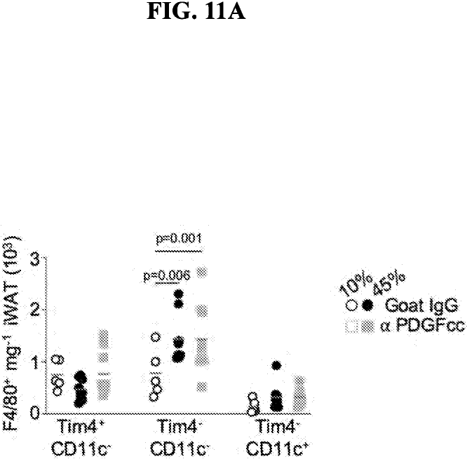

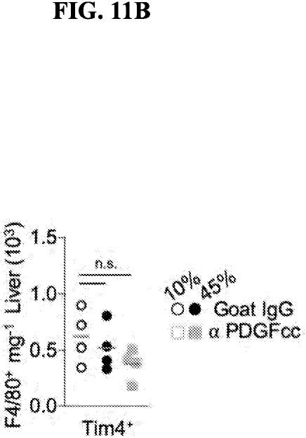

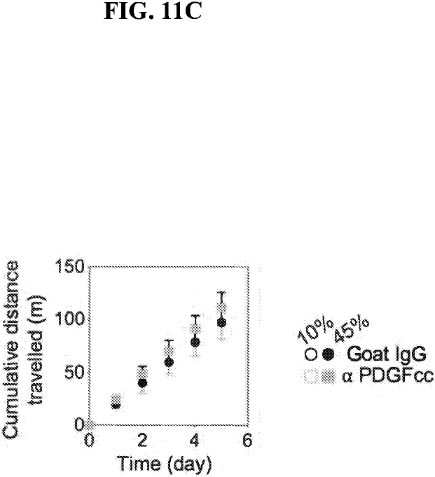

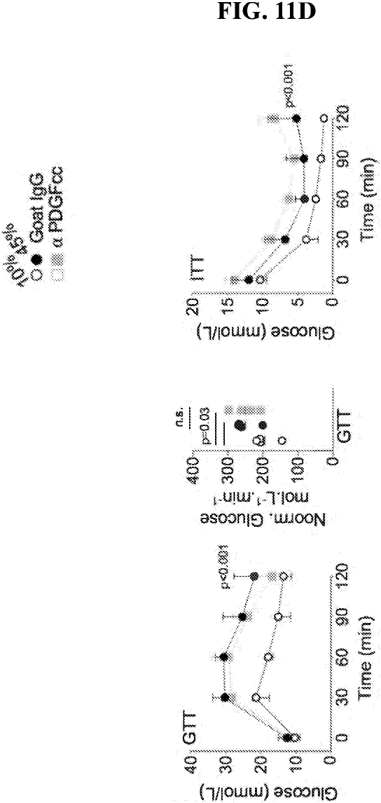

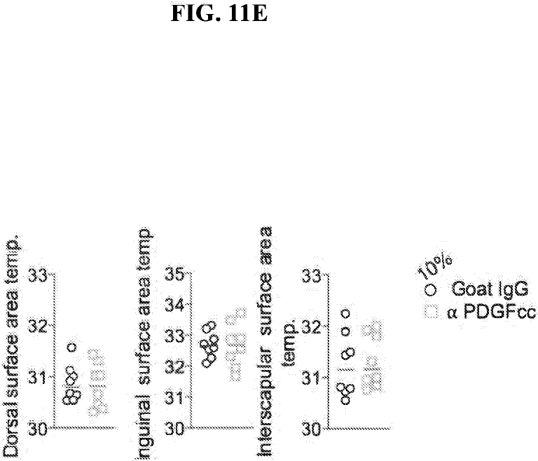

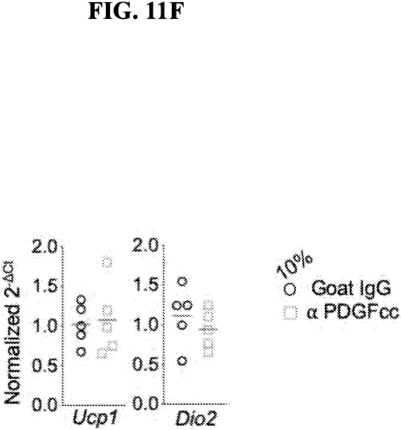

The above data suggests that PDGFcc is important for lipid storage in murine adipocytes. To investigate this hypothesis in vivo, mice were treated with anti-PDGFcc neutralizing antibodies (AF1447, 10 µg/mouse, ip, thrice a week). Anti-PDGFcc antibodies do not deplete macrophages (FIGS. 11A-11B). However, mice placed on a high-fat diet (45% kCal from fat, 4.73 kCal/g) failed to gain weight (FIG. 5A) and remained lean as white adipose tissue mass (FIG. 5B) and adipocyte size (FIGS. 5C-5D) were reduced almost to control levels. Food intake, fecal caloric density, and daily activity were similar in treated and untreated mice (FIGS. 5E-5F, FIG. 11C), but energy expenditure was increased in the treated group (FIG. 5G). Increased metabolic activity of white and brown adipose tissues in anti-PDGFcc treated mice on a high-fat diet was also evidenced by increased surface body temperature (FIG. 5H), as well as elevated Ucp1 and Dio2 transcripts in the the iBAT (FIG. 51). Importantly, anti-PDGFcc did not prevent the metabolic complications associated with lipid-rich diet including hepatic steatosis (FIGS. 5J-5K), liver inflammation (FIG. 5L) and insulin resistance (FIG. 11D), which are mediated by Ccr2-dependent inflammatory monocyte/macrophages. Of note, in mice fed a control diet (10% kCal from fat, 3.85 kCal/g), anti-PDGFcc antibodies did not detectably decrease mouse weight (FIG. 5A), fat tissue mass (FIG. 5B), or adipocyte size (FIGS. 5C-5D) and do not detectably increase thermogenesis or Ucp1 and Dio2 transcripts in the iBAT (FIGS. 11E-11F). These data are consistent with results from the ex vivo experiments above, suggesting that PDGFcc controls a balance between lipid storage and thermogenesis in the adipose tissue during development and in conditions of “stress,” i.e., increased food intake, without affecting systemic inflammation and ectopic deposition of lipids mediated by HSC-derived monocytes/macrophages. Accordingly, these results demonstrate that PDGFcc antagonists are useful in methods for treating or preventing lipid accumulation in adipose tissue in a subject in need thereof. In addition, these results also demonstrate that PDGFcc or PDGFcc agonists are useful in methods for treating or preventing diet-induced lipid accumulation in adipose tissue and obesity in a subject in need thereof.

### Example 9: Lipid-rich Diet Up-Regulates Pdgfcc Expression by Resident Macrophages in Adult Adipose Tissue

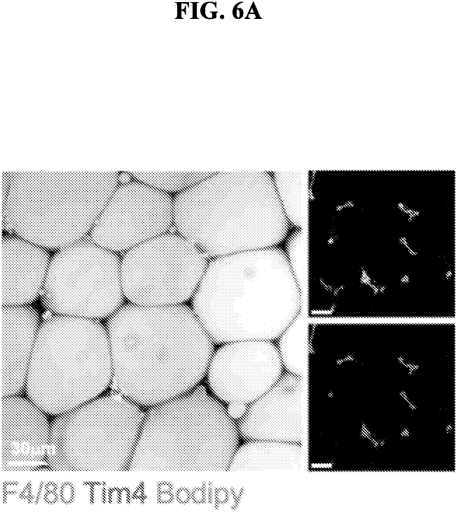

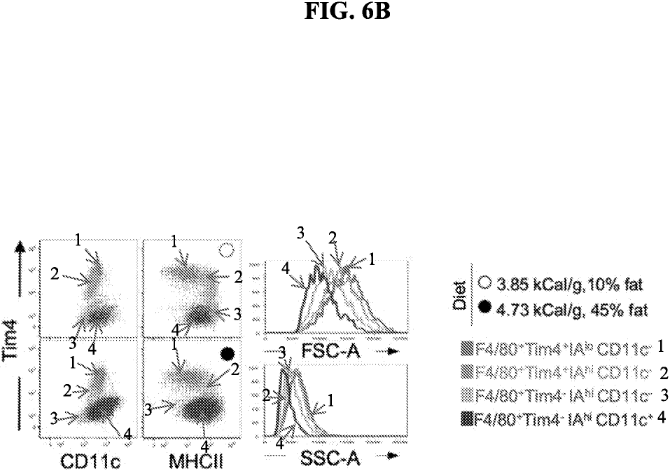

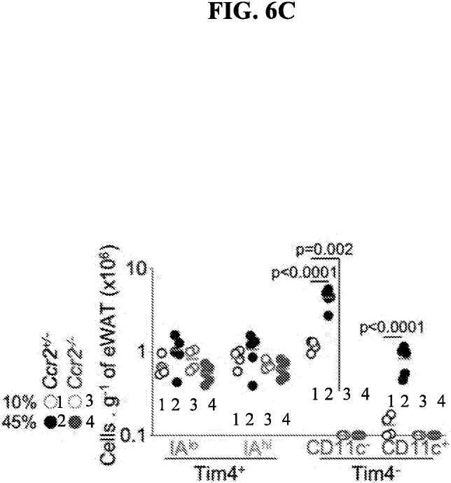

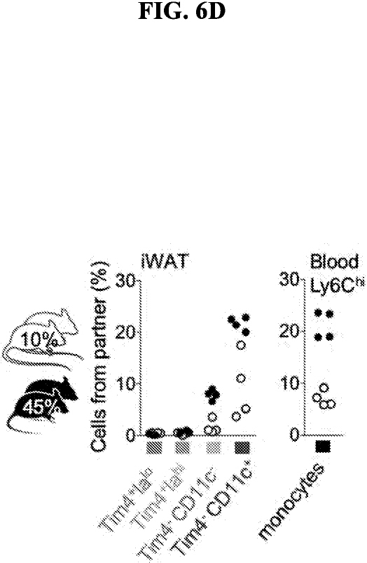

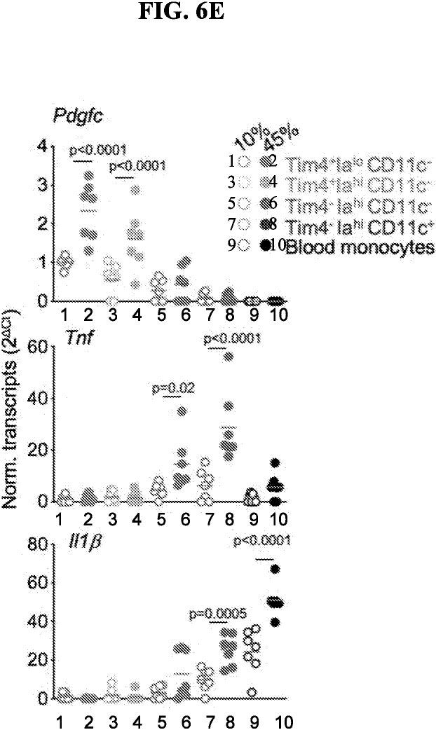

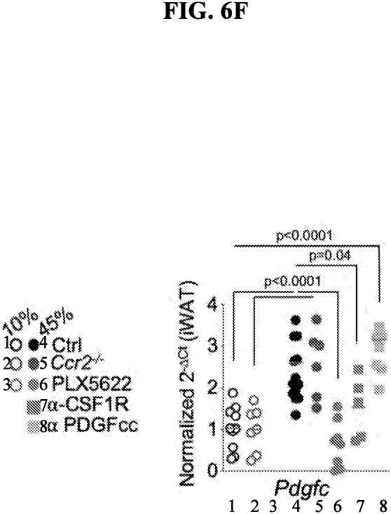

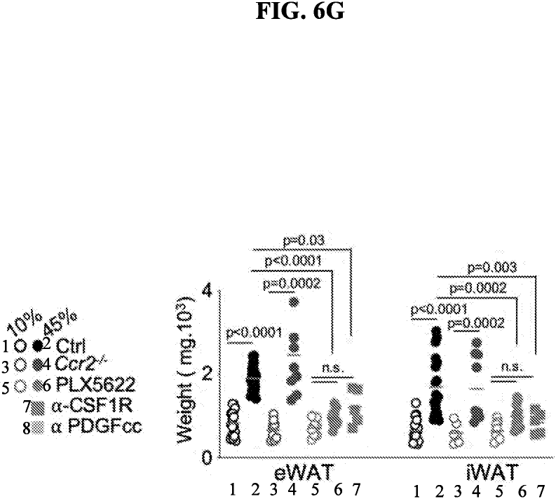

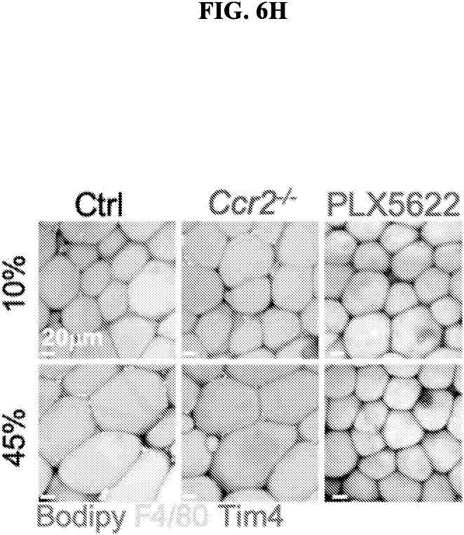

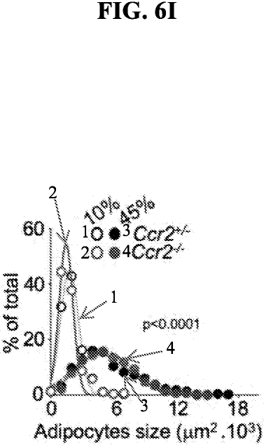

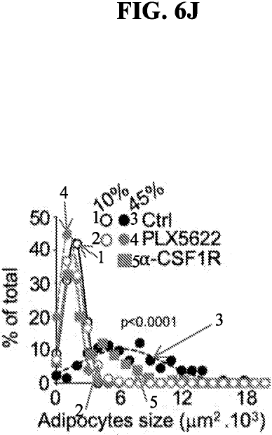

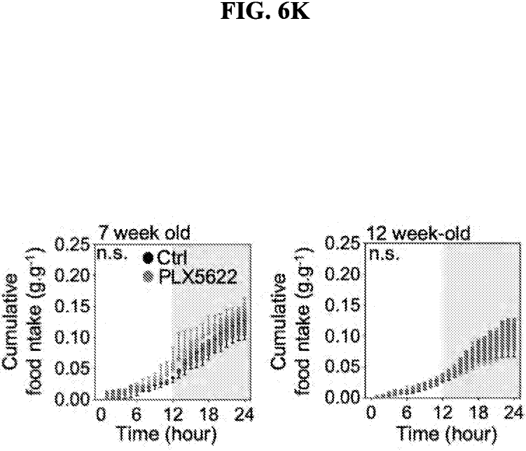

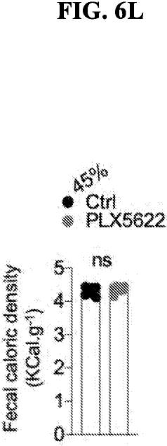

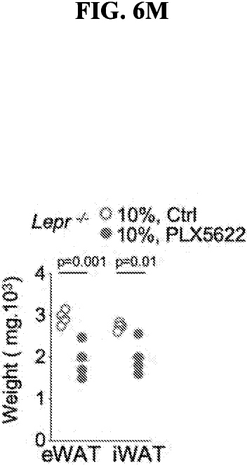

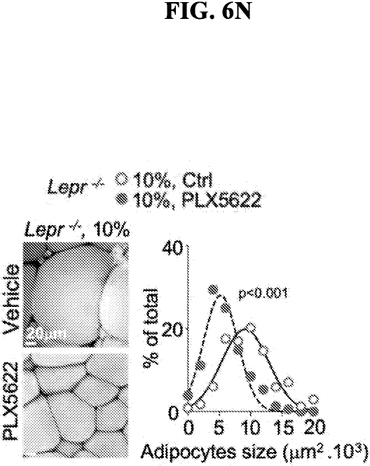

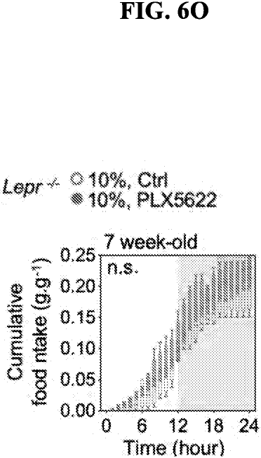

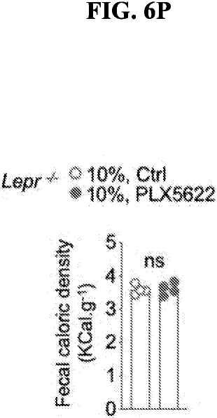

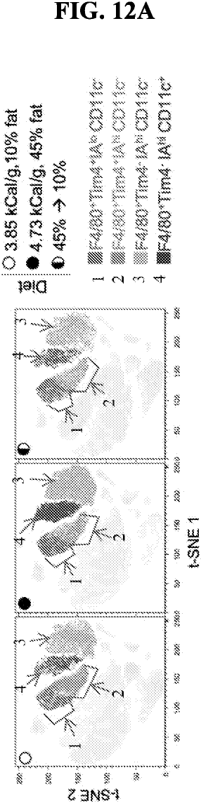

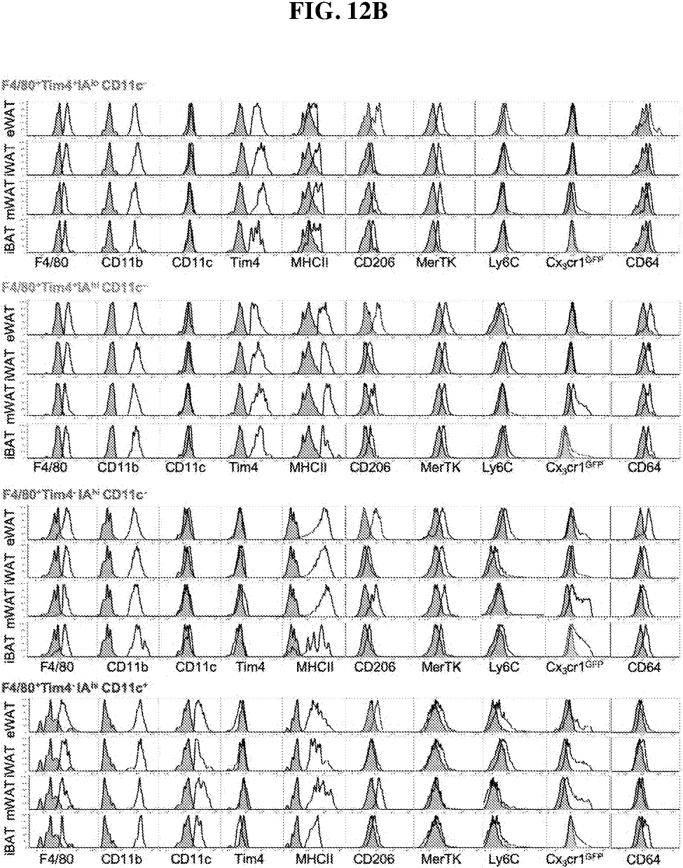

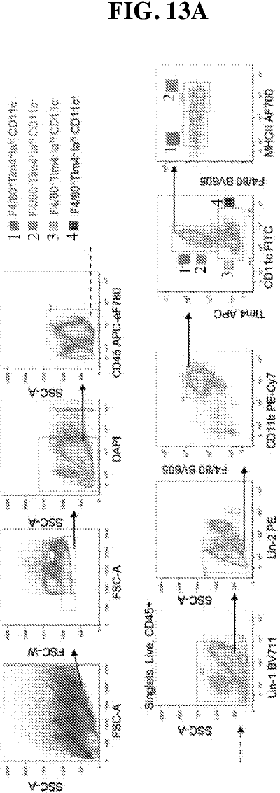

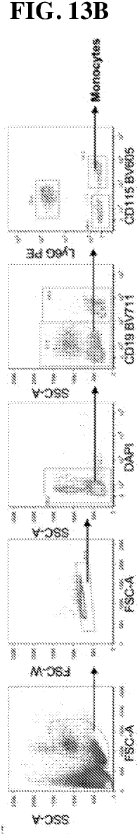

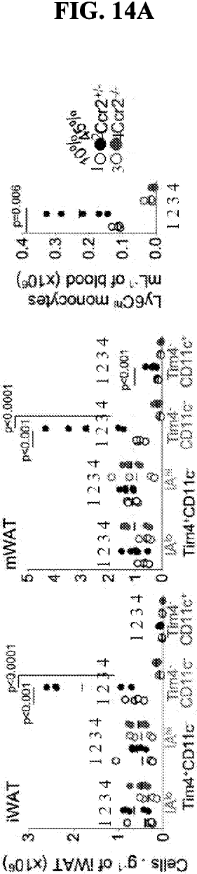

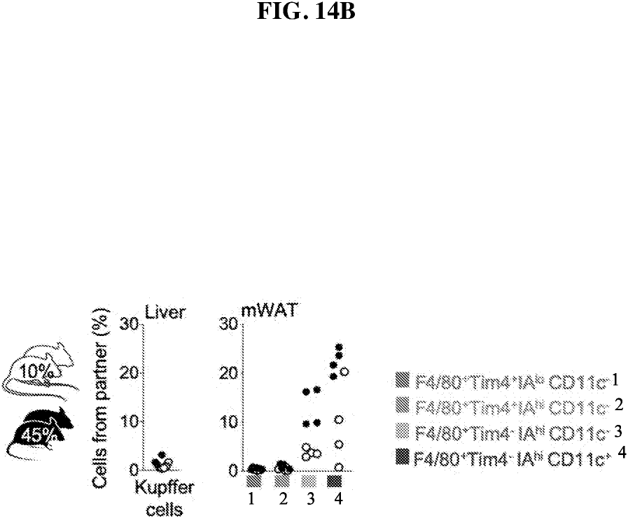

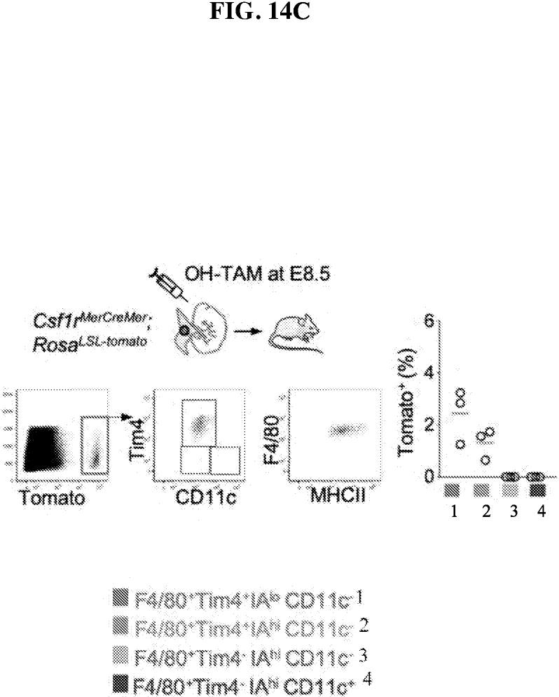

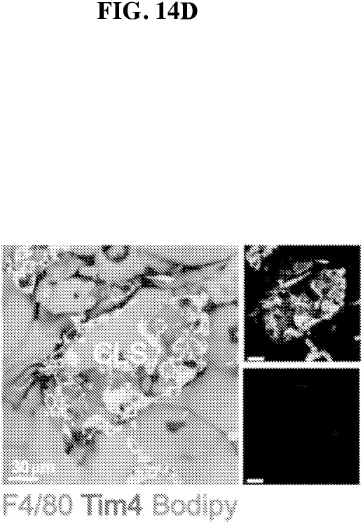

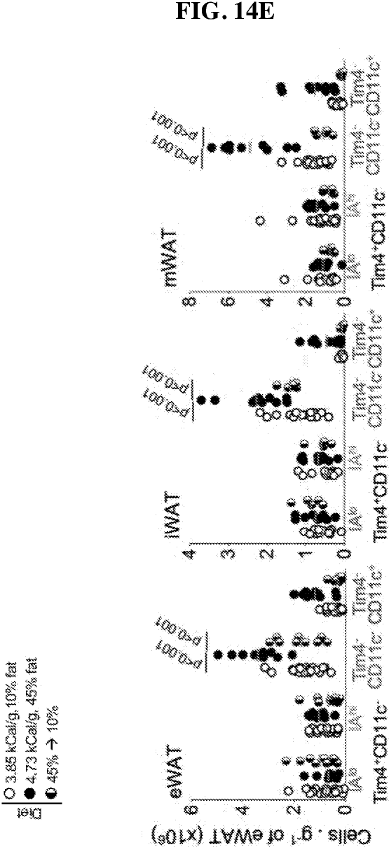

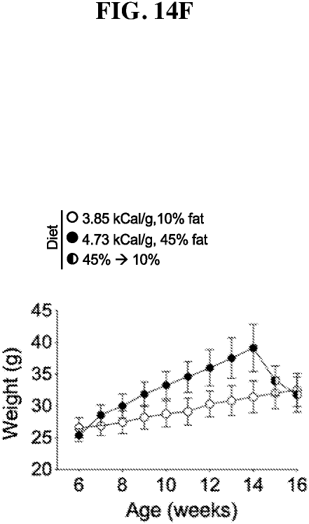

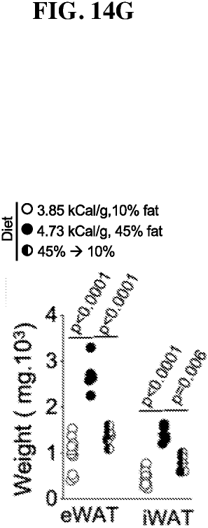

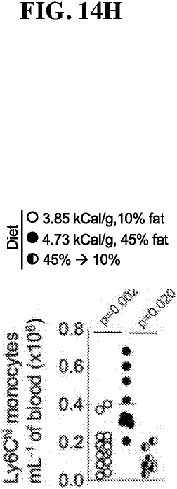

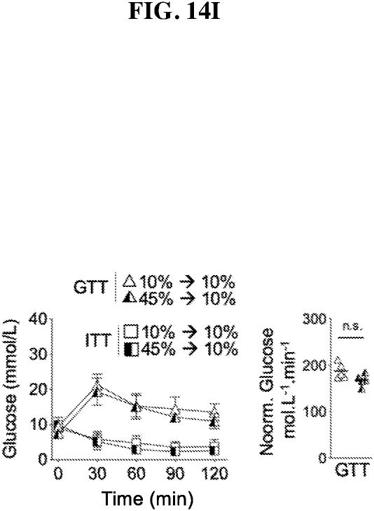

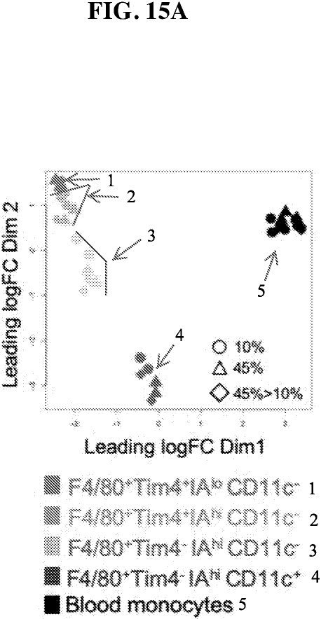

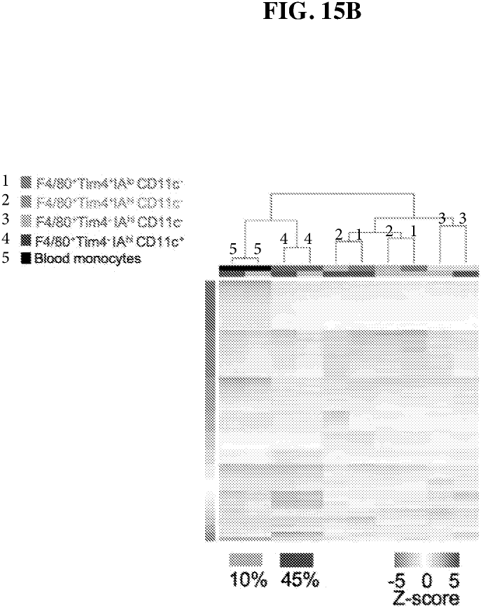

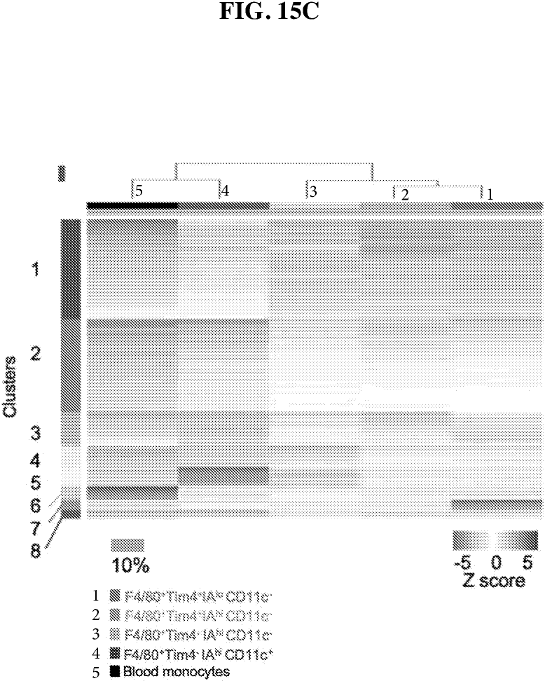

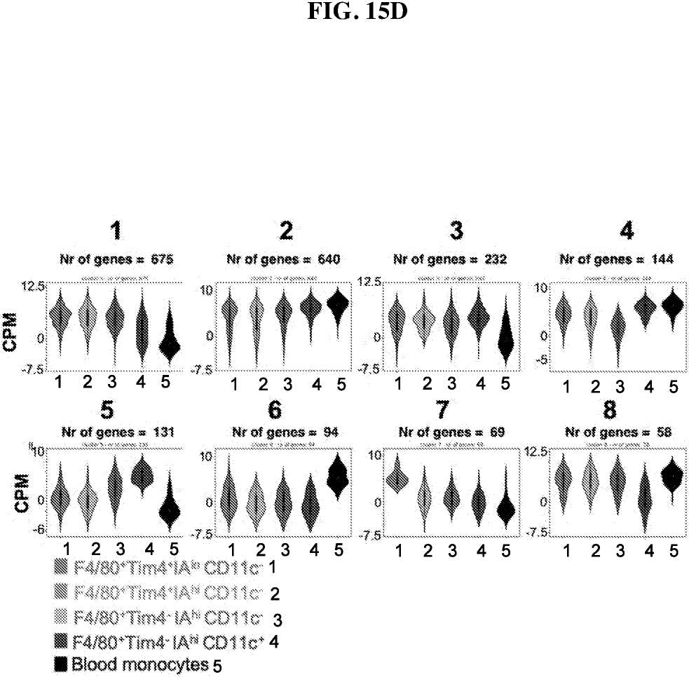

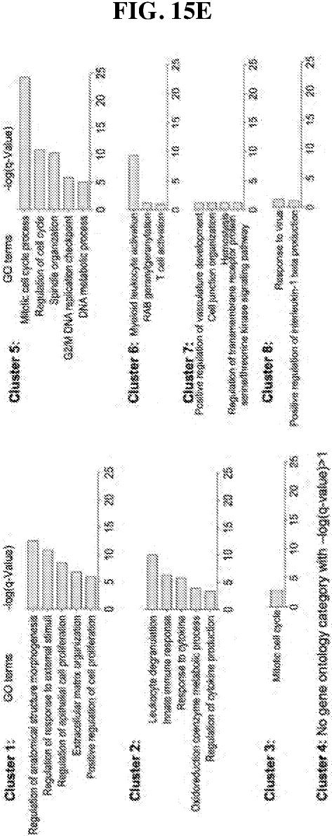

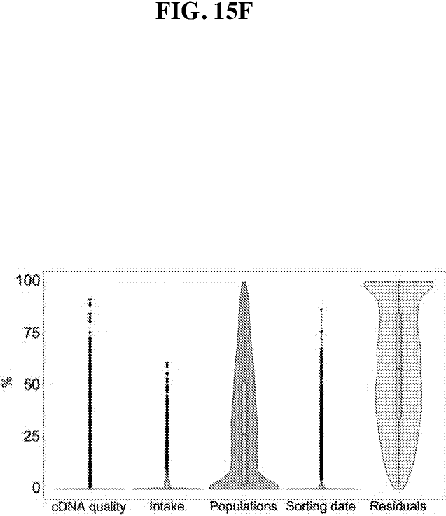

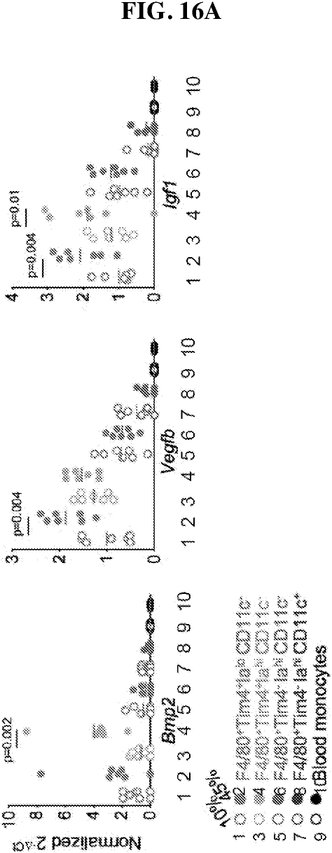

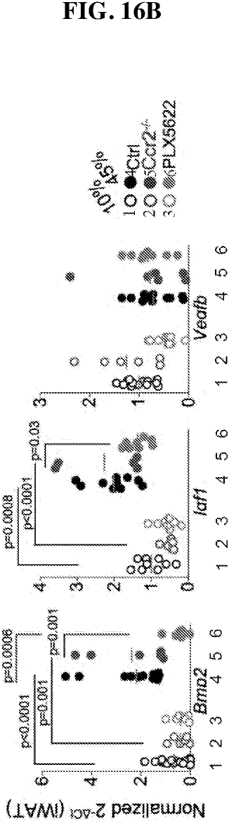

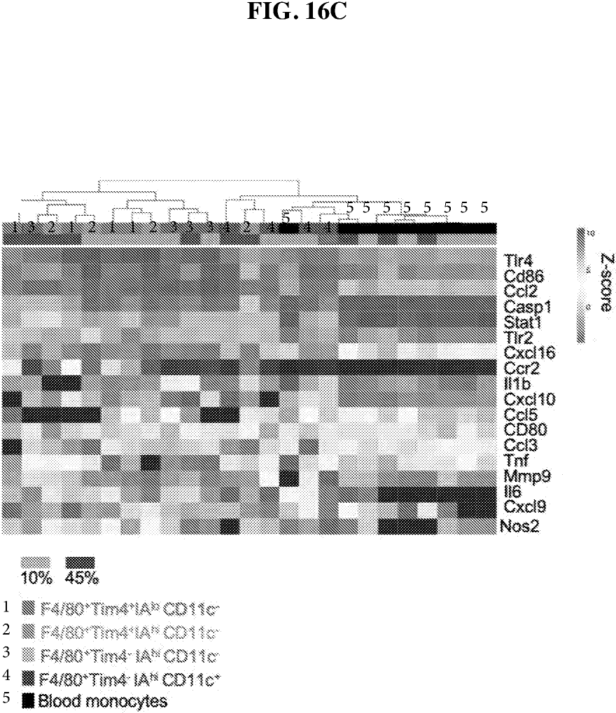

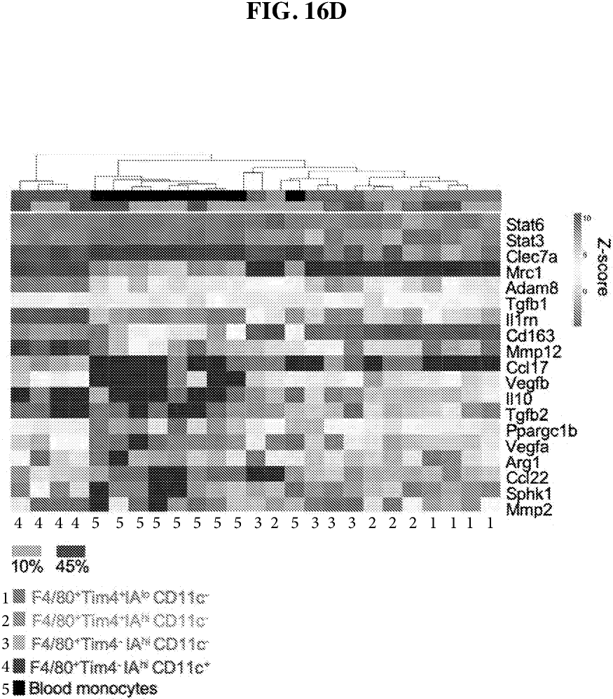

Fluorescence microscopy and flow cytometry analyses of F4/80⁺ cells in visceral and subcutaneous white adipose tissue from adult mice (8 to 14 weeks) indicated that large resident F4/80⁺ Tim4⁺ macrophages persist in the adipose tissue of lean and obese adult mice (FIGS. 6A-6B, FIGS. 12-14). These F4/80⁺ Tim4⁺ macrophages were present in similar numbers (~2×10⁶.g⁻¹ of tissue) in the adipose tissue of lean and obese animals, from either Ccr2-deficient mice or littermate controls (FIG. 6C, FIG. 14A). They did not exchange between mice in the course of 8-week long parabiosis (FIG. 6D, FIG. 14B), and they were labeled by Cre-mediated inducible lineage tracing of yolk sac EMPs (FIG. 14B) indicating that resident macrophages persist in adult adipose tissue. Transcriptional analysis indicated that these resident F4/80⁺ Tim4⁺ macrophages express genes associated with homeostatic processes (Cluster 1, 7 FIG. 15). A qPCR analysis of FACS-sorted cells confirmed that F4/80⁺ Tim4⁺ macrophages were the main source of adipogenic growth factors, including Pdgfc, the expression of which was further upregulated on high fat diet (FIG. 6E, FIG. 16). In contrast, small F4/80⁺ Tim4⁻ monocyte/macrophages accumulated in fat pads of obese wt mice, forming crown-like structures (FIGS. 14A-14B) but were missing from white adipose tissue of Ccr2-deficient mice (FIG. 6C). They exchanged between parabiotic mice (FIG. 6D, FIG. 14B), and were not labeled by Cre-mediated inducible lineage tracing of yolk sac EMPs (FIG. 14C), suggesting they correspond to HSC-derived monocyte/macrophages or monocyte-derived DCs. Transcriptional analysis indicated that these Tim4⁻ monocyte/macrophages highly express genes associated with innate immune response and cell proliferation (Clusters 2 and 5 FIG. 15) and qPCR analysis confirmed that they, and blood monocytes, are a source of TNF and IL-1b in mice under high fat diet (FIG. 6E, FIG. 16). Upon return to a standard diet (10%), small F4/80⁺ Tim4⁻ monocyte/macrophages numbers returned to baseline within 2 weeks (FIG. 14E), as the mice returned to a lean state (FIGS. 14F-14I).

These data indicate that resident F4/80⁺ Tim4⁺ and HSC-derived F4/80⁺ Tim4⁻ cells share the same tissue in adult adipose tissue, but are nonetheless characterized by distinct phenotypes and cellular dynamics. Gene expression was consistent with an inflammatory role for Tim4⁻ monocyte/macrophages, while F4/80⁺ Tim4⁺ resident macrophage up-regulated the expression of adipogenic growth factors such as Pdgfc when mice were fed a lipid-rich diet. It is of note that these cells cluster by population rather than diet in multidimensional scaling and hierarchical clustering of differentially expressed genes (FIG. 15). Additionally, the analysis of expression of genes associated with M1 or M2 polarization of macrophages did not indicate a clear macrophage polarization in response to dietary changes (FIGS. 16C-16D).

### Example 10: Deletion of Macrophages in Adult Wt and Db/db Mice Abolishes Pdgfc Up-Regulation and Adipocytes Hypertrophy

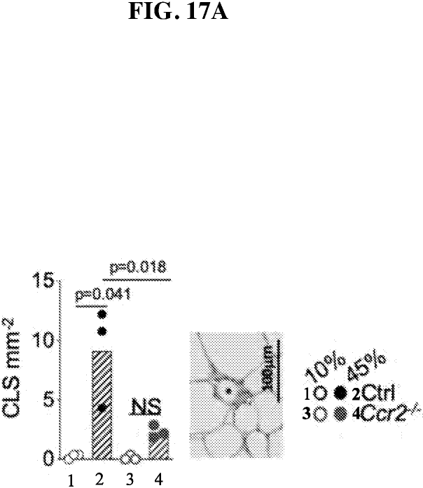

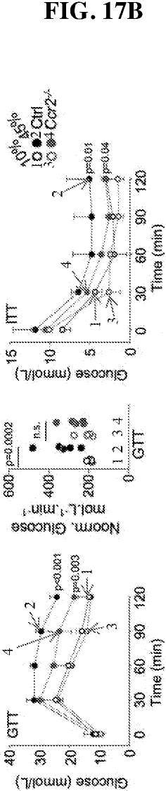

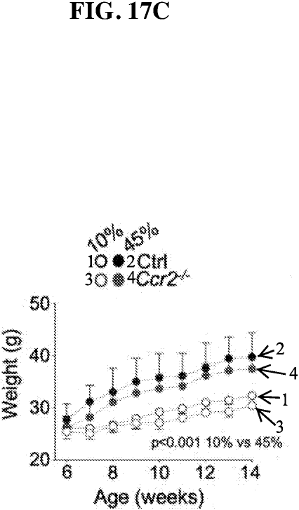

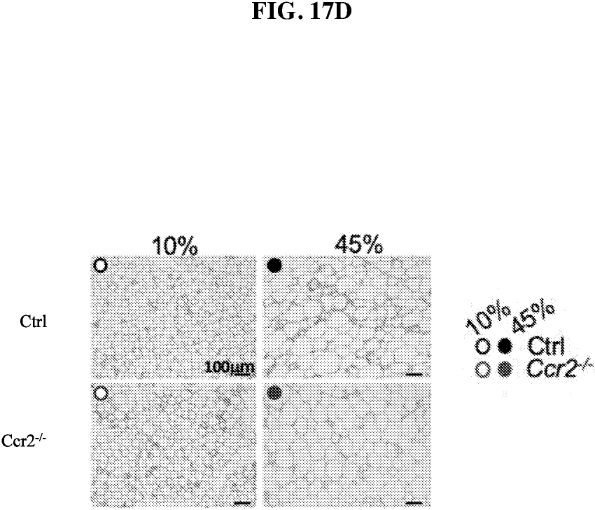

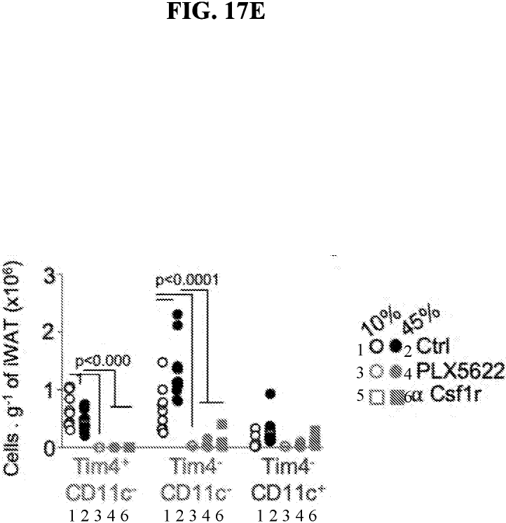

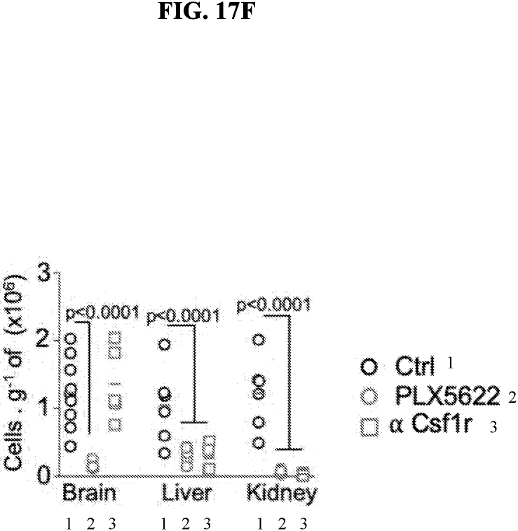

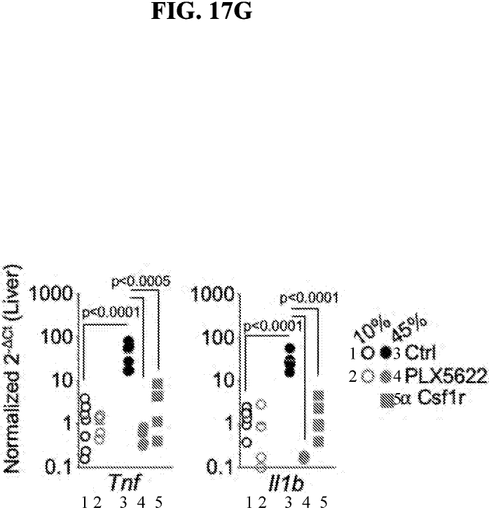

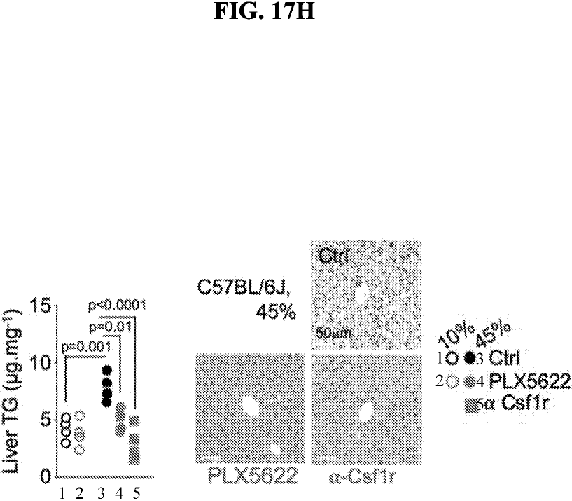

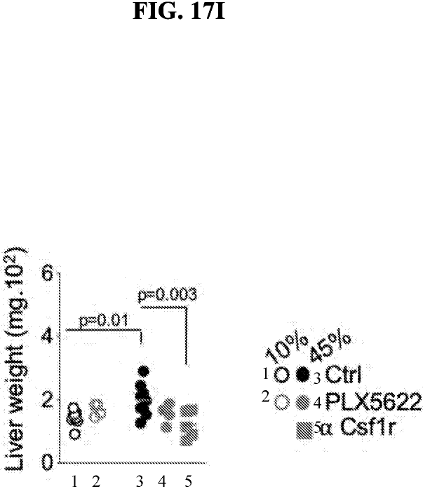

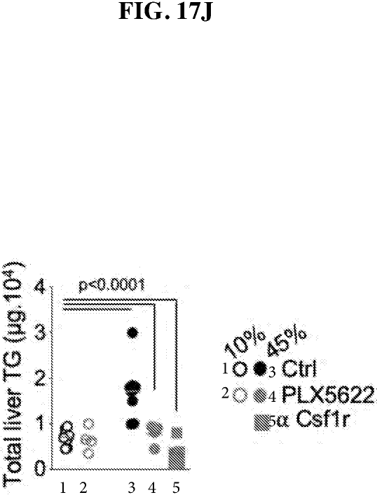

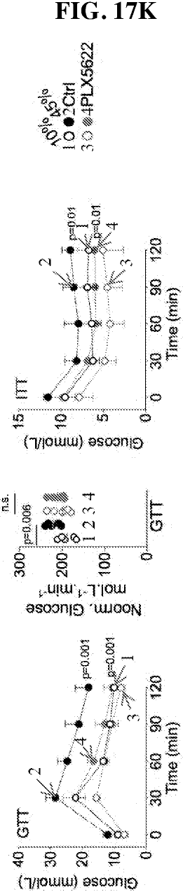

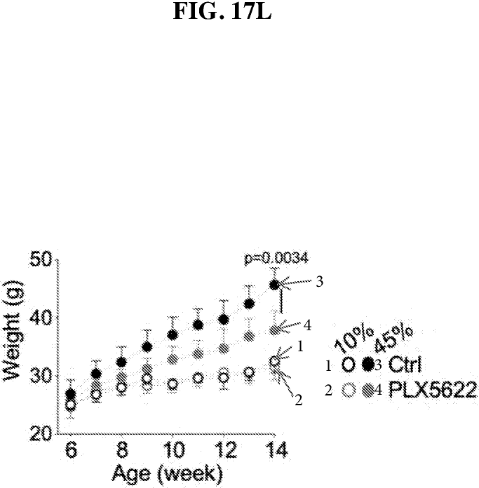

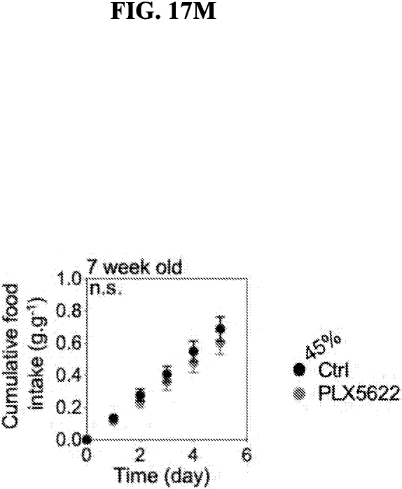

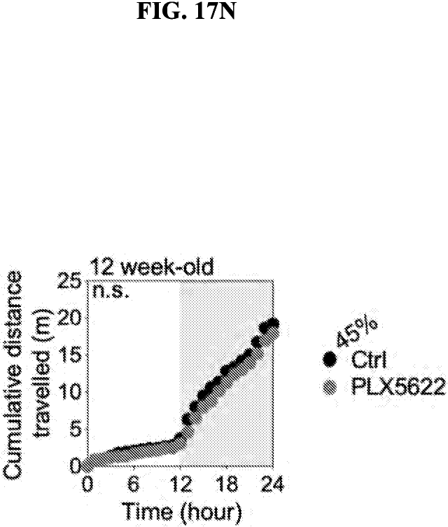

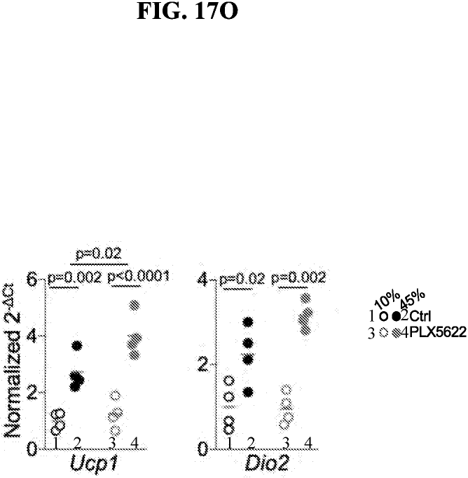

To confirm that resident Tim4+ macrophages are a source of PDGFcc required for adipose tissue expansion in obese mice, the effect of Ccr2-deficiency and CSF1R blockade was compared. Ccr2-deficient mice fed a lipid rich diet are protected against hepatic steatosis, monocyte recruitment, inflammation, and the metabolic syndrome. In accordance with these previous observations, Ccr2-deficient mice fed a lipid rich diet were protected against accumulation of small F4/80⁺ Tim4⁻ monocyte/macrophages in adipose tissue (FIG. 6C), CLS formation, and the metabolic syndrome (FIGS. 17A-17B). However, Ccr2-deficient mice were obese to the same extent as control mice (FIG. 17C). Tim4+ adipose tissue macrophages (FIG. 6C), Pdgfc expression in fat tissue (FIG. 6F), fat mass (FIG. 6G), and adipocyte hypertrophy (FIGS. 6H-6L, FIG. 17D) were no different in Ccr2-deficient mice and control littermates.

Mice treated with the CSF1R inhibitor, PLX5622 (1,200 mg PLX5622/kg of food), lacked both F4/80⁺ Tim4⁻ and Tim4⁺ macrophages (FIGS. 17E-17F). They were also protected against TNF/IL1 production, hepatic steatosis, and the metabolic syndrome (FIGS. 17G-17K). PLX5622 treated mice also gained less weight than controls when fed a high fat diet (FIG. 17L), they did not increase Pdgfcc expression in their fat pads (FIG. 6F), their visceral and subcutaneous white adipose tissue mass did not increase (FIG. 6G), and they did not develop adipocyte hypertrophy (FIGS. 6H-6J). Despite these phenotypes, food intake, fecal caloric density, or ambulatory activity were no different in PLX5622-treated and control mice (FIGS. 6K-6L, FIGS. 17M-17N). Because macrophage depletion protects mice against inflammation and the metabolic syndrome, comparison of energy expenditure between treated and control mice will not inform on the effect of PDGFcc or resident macrophages alone, nevertheless an increased Ucpl and Dio2 expression in the iBAT of PLX5622-treated mice was observed, compatible with increased catabolic activity (FIG. 170). CSF1R blockade thus appears to recapitulate both the effects of Ccr2 deficiency and of PDGFcc blockade. Importantly, these data confirm that resident macrophages are a major source of Pdgfc in adult adipose tissue, and are required for increased Pdgfc and storage of excess lipid in adipocytes during lipid-rich diet feeding.

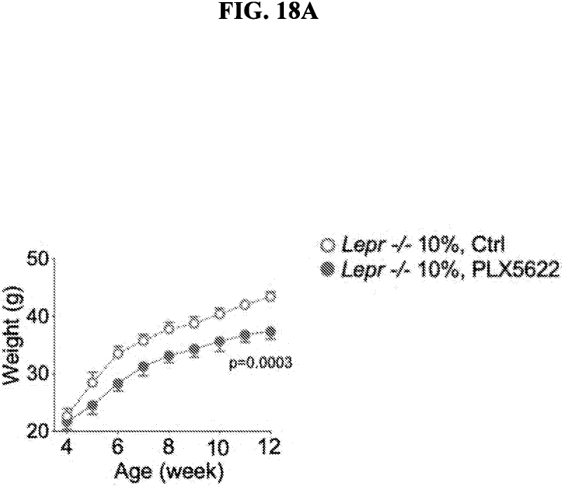

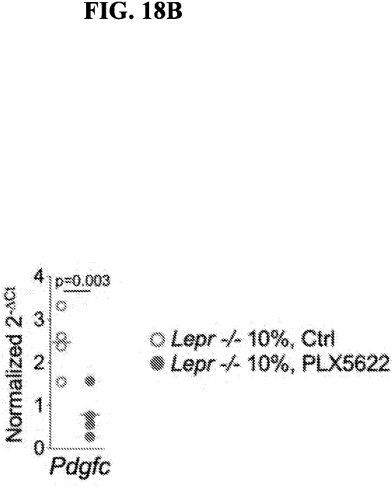

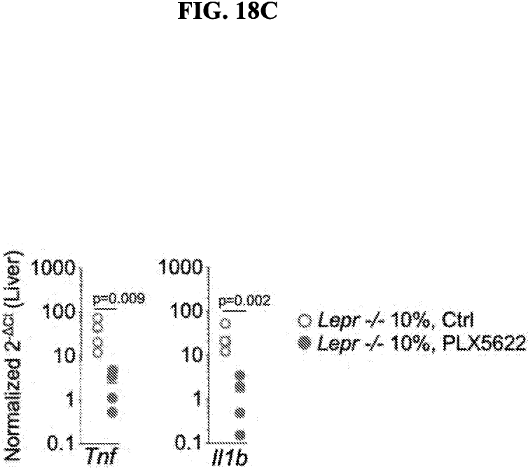

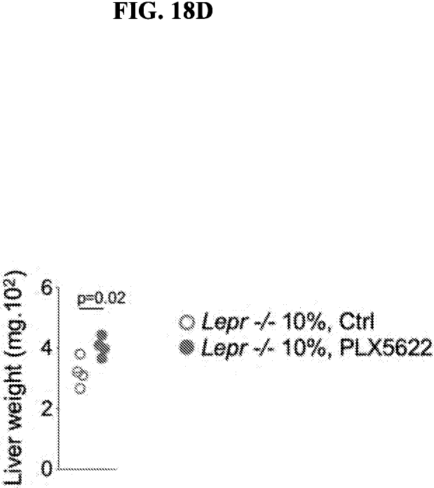

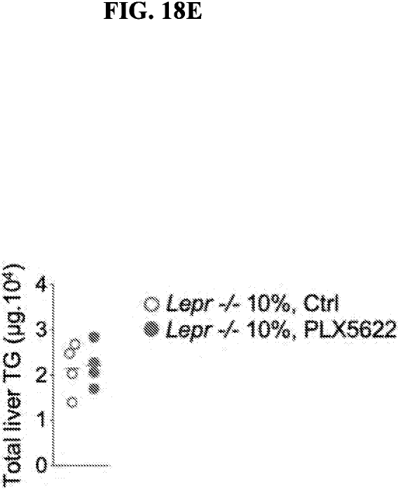

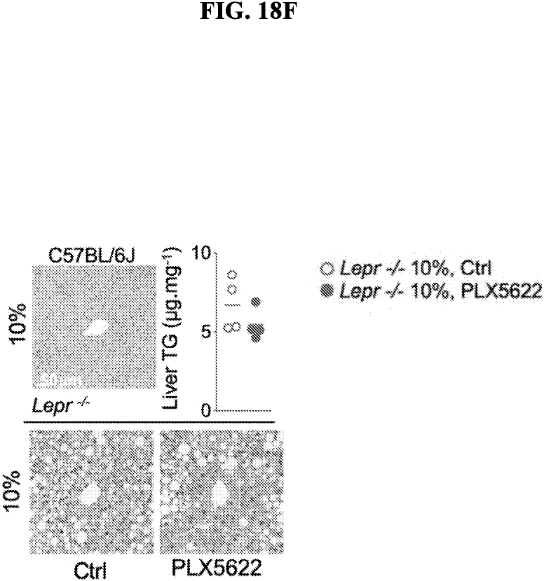

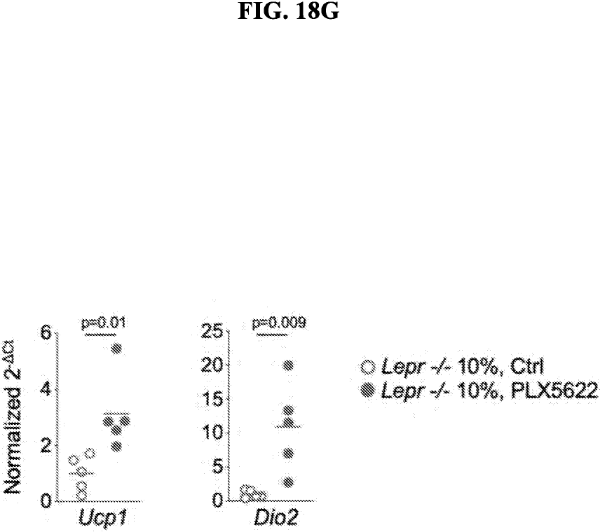

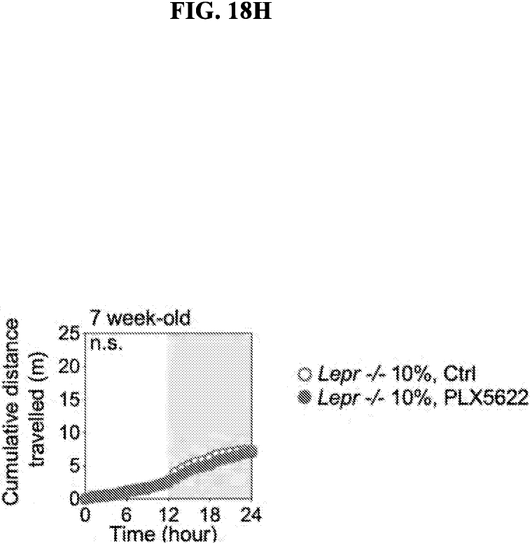

To control for off-target effects of the tyrosine kinase inhibitor, PLX5622, mice were also treated with a blocking anti-CSF1R antibody (AFS98, 50 mg/kg, ip, three times a week). AFS98 treatment depleted adipose tissue macrophage -but not microglia-(FIGS. 17E-17F), and reduced Pdgfc expression (FIG. 6F), adipose tissue mass (FIG. 6G), and adipocyte size in comparison to controls (FIG. 6J). To further control for possible effects of systemic macrophage depletion on leptin signaling, leptin-receptor deficient mice (Lepr^{-/-}, db/db) that spontaneously develop obesity were analyzed. Again, PLX5622 treatment decreased weight gain (FIG. 18A), Pdgfc expression in adipose tissue (FIG. 18B), adipose tissue mass (FIG. 6M), adipocyte size (FIG. 6N), as well as liver inflammation (FIGS. 18C-18F). PLX5622 treatment in Lepr^{-/-} mice also increased Ucpl and Dio2 expression (FIG. 18G), but did not detectably modify food intake (FIG. 6O), fecal caloric density (FIG. 6P), or ambulatory activity (FIG. 18H) in comparison to untreated controls.

### Discussion

As described herein, PDGF/VEGF family growth factors produced by resident macrophages associated with adipose tissue are required for efficient lipid storage in fat cells from Drosophila larva, early post-natal development in mice, and in conditions of excess intake in adult mice. Specifically, the data presented herein support the hypothesis that large Tim4⁺ yolk-sac derived adipose tissue resident macrophages control fat storage in white adipose tissue in development and obesity in a paracrine manner, via diet-regulated production of PDGFcc.

Macrophage developmental and phenotypic heterogeneity in mice has long been suspected to underlie specialized functions attributable to different macrophage cell types. In contrast to resident macrophages, HSC-derived Tim4⁻ monocyte/macrophages are neither required, nor sufficient, for fat storage in white adipose tissue, while they are required in obese animals to mediate TNF and IL-1 production, hepatic steatosis, and metabolic syndrome. Accordingly we find that resident macrophages are not sufficient to mediate systemic inflammation, ectopic deposition of lipids, and metabolic syndrome. Therefore, our data do not support a model where inflammation and insulin resistance in mice would be controlled by the reprogramming of resident macrophages from a homeostatic into a pro-inflammatory role in the setting of metabolic stress. Instead, distinct developmental subsets, i.e., resident macrophages and recruited HSC derived monocyte/macrophages perform distinct functions in the adipose tissue ‘niche’.

The function of resident macrophages, i.e., promoting the expansion of energy stores in specialized fat cells, appears to be evolutionarily conserved in mice and Drosophila. It is proposed that macrophages and specialized fat-storing cells may constitute a functional unit in metazoans, where macrophages sense the organism’s nutritional state and signal increased lipid intake to adipocytes through regulated paracrine production of PDGF/VEGF family growth factors. The results presented herein suggest that PDGFcc achieves this effect by limiting catabolic activity of adipocytes at the tissue and organismal level. Drosophila do not have fibroblasts or endothelial cells in the fat body, and Pvr silencing in fat cells phenocopies Pvf3 silencing in hemocytes, suggesting that the interaction between macrophages and fat cells may be a direct one in flies.

Further mechanistic studies in mice directed at investigating the roles of macrophage-derived PDGFcc and other growth factors on endothelium and stromal cells for the purpose of fat storage are warranted since for example a role of endothelial cells cannot be eliminated in mice. More importantly, PDGF-receptor signaling plays complex and at time opposing roles in lipid storage, and the downstream molecular mechanisms involved in the control of lipid storage and energy expenditure by PDGF signaling in fat cells are not characterized.

The present disclosure provides a cellular “dissection” of macrophage functions in lipid metabolism and identifies a mechanism that controls the expansion of fat stores in metazoans. These data point towards the feasibility of pharmacological approaches that would aim at preventing pathological loss of fat stores i.e., cachexia, or gain of lipid stores as is the case in morbid obesity.

### Example 11: Methods for Debulking a Liposarcoma Tumor to Facilitate Surgical Removal of the Liposarcoma Tumor

Effects of a PDGFcc antagonist, an anti-PDGFcc antibody, or an active fragment or a homolog thereof, on a liposarcoma tumor will be tested with xenograft animal models of liposarcoma. Suitable xenograft animal models of liposarcoma include, but are not limited to, those described by Codenotti S., et al., Onco Targets Ther 12: 5257-5268 (2019), which is incorporated by reference herein in its entirety. Xenograft animal models of liposarcoma will be treated with injection of PBS control or a therapeutically effective amount of PDGFcc antagonist (or an anti-PDGFcc antibody, or an active fragment or a homolog thereof) e.g., i.p., i.v., i.m, or intratumoral; e.g., once, once daily for 1 week or more, or any other treatment regimen suitable for achieving the therapeutic effect of reducing the liposarcoma tumor volume. Tumor volumes will be determined throughout the study by, e.g., using a digital caliper and the formula: tumor volume = ½ (length x width²) where the greatest longitudinal diameter is the length of the tumor and the greatest transverse diameter is the width. At study termination, the animal will be euthanized, e.g., by CO₂.

It is expected that subjects treated with compositions of the present technology comprising an effective amount of a PDGFcc antagonist, an anti-PDGFcc antibody, or an active fragment or a homolog thereof, will exhibit a decreased liposarcoma tumor volume as compared to untreated controls. Accordingly, these results will demonstrate that compositions of the present technology comprising an effective amount of a PDGFcc antagonist, an anti-PDGFcc antibody, or an active fragment or a homolog thereof, are effective in methods for debulking a liposarcoma tumor.

### Example 12: Methods for Treating or Preventing Cachexia

Effects of a PDGFcc agonist (e.g., PDGFcc or PDGFcc peptide), or an active fragment or a homolog thereof, or VEGFb, or VEGFb peptide, or an active fragment or a homolog thereof, on cachexia will be tested with a cachexia mouse model. The cachectic mice models will be generated using KPC tumor cells, which are derived from a primary culture of pancreatic tumor cells of the genetically engineered mouse model of PDAC (K-rasLSL^{G12D/+}; p53^{R172H/+}; Pdx-Cre (KPC)). Briefly, 0.7 × 10⁶ KPC tumor cells in 200 µl PBS will be injected subcutaneously into the flank of C57bl/6J mice. The generated cachexia mice models as well as the controls will be treated with injection via a suitable route of administration (e.g., i.p., i.v., i.m) of PBS control, or therapeutically effective amounts of a PDGFcc agonist, PDGFcc, PDGFcc peptide, VEGFb, or VEGFb peptide. Mice are then sacrificed 5 weeks after the injection of KPC tumor cells, when tumor volume is approaching 5 mm of radius. Cachexia and fat tissue loss as compared to control mice will be evaluated by (i) magnetic resonance images to determine body composition, (ii) by acquiring fat tissue weight, and (iii) by whole-mount imaging to evaluate adipocyte size.

It is expected that cachectic subjects treated with a therapeutically effective amount of a PDGFcc agonist, PDGFcc, PDGFcc peptide, VEGFb, or VEGFb peptide will exhibit an observable and/or measurable increase in fat body mass and lipid storage by adipocytes as compared to untreated controls. Accordingly, these results will demonstrate that compositions of the present technology comprising an effective amount of a PDGFcc antagonist, an anti-PDGFcc antibody, a VEGFb, or a VEGFb peptide, or an active fragment or a homolog thereof, are effective in methods for treating or preventing cachexia in a subject in need thereof.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

What is claimed is:

1. A method for treating or preventing lipid accumulation in adipose tissue in a subject in need thereof, comprising administering to the subject an effective amount of a PDGFcc antagonist, an active fragment, or a homolog thereof.

2. The method of claim 1, wherein the PDGFcc antagonist is an anti-PDGFcc antibody, or a fragment thereof.

3. The method of claim 1 or claim 2, comprising selecting for treatment a subject with at least one disease or condition selected from the group consisting of obesity, metabolic syndrome, and hyperlipidemia.

4. A method for treating or preventing obesity in a subject in need thereof, comprising administering to the subject an effective amount of a PDGFcc antagonist, an active fragment, or a homolog thereof.

5. The method of claim 4, wherein the PDGFcc antagonist is an anti-PDGFcc antibody, or a fragment thereof.

6. The method of claim 4, comprising administering to the subject an effective amount of a combination therapy of: i) a PDGFcc antagonist, an active fragment, or a homolog thereof; and ii) at least one of a CCR2 antagonist or an anti-CCR2 antibody.

7. The method of claim 5, comprising administering to the subject an effective amount of a combination therapy of: i) an anti-PDGFcc antibody, an active fragment, or a homolog thereof; and ii) at least one of a CCR2 antagonist or an anti-CCR2 antibody.

8. The method of any one of claims 4-7, wherein the obesity is diet-induced or genetic.

9. The method of any one of claims 4-7, wherein the obesity is characterized by adipocyte hypertrophy.

10. A method for treating or preventing lipodystrophy in a subject in need thereof, comprising administering to the subject an effective amount of PDGFcc or a PDGFcc agonist, an active fragment, or a homolog thereof.

11. The method of claim 10, comprising administering to the subject an effective amount of a combination therapy of: i) PDGFcc or a PDGFcc agonist, an active fragment, or a homolog thereof; and ii) at least one of a CCR2 antagonist.

12. The method of claim 10, comprising administering to the subject an effective of amount of: i) PDGFcc or PDGFcc agonist, an active fragment, or a homolog thereof; and ii) an anti-CCR2 antibody.

13. The method of claim any one of claims 10-12, further comprising administering an effective amount of VEGFb, an active fragment, or a homolog thereof.

14. The method of any one of claims 10-13, wherein the lipodystrophy is hereditary.

15. The method of any one of claims 10-13, wherein the lipodystrophy is associated with HIV infection.

16. The method of any one of claims 10-13, wherein the lipodystrophy is associated with an HIV medication.

17. The method of claim 16, wherein the HIV medication is thymidine analogue nucleoside reverse transcriptase inhibitor.

18. The method of claim 16, wherein the HIV medication is zidovudine (AZT) or stavudine (d4T).

19. The method of claim 16, wherein the HIV medication is an HIV-1 protease inhibitor or nucleoside reverse transcriptase inhibitors (NRTI).

20. A method for treating or preventing cachexia in a subject in need thereof, comprising administering to the subject an effective amount of PDGFcc or a PDGFcc agonist, an active fragment, or a homolog thereof.

21. The method of claim 20, comprising administering an effective amount of a combination therapy of: i) PDGFcc or PDGFcc agonist, an active fragment, or a homolog thereof; and ii) at least one of a CCR2 antagonist.

22. The method of claim 20, comprising administering an effective of amount of: i) PDGFcc or PDGFcc agonist, an active fragment, or a homolog thereof; and ii) an anti-CCR2 antibody.

23. The method of any one of claims 20-22, further comprising administering an effective amount of VEGFb, an active fragment, or a homolog thereof.

24. The method of any one of claims 20-23, further comprising separately, sequentially, or simultaneously administering an additional treatment to the subject.

25. The method of claim 24, wherein the additional treatment comprises administration of a therapeutic agent.

26. The method of claim 25, wherein the therapeutic agent is selected from the group consisting of: prednisolone, methylprednisolone, dexamethasone, megestrol acetate, medroxyprogesterone, dronabinol, cyproheptadine, metoclopramide, cisapride, nandrolone decanoate, fluoxymesterone, testosterone, oxandrolone, enobosarm, ghrelin (anamorelin), hydrazine sulfate, pentoxifylline, lisofylline, thalidomide, anti-IL-6 antibody, IL-12, branched-chain amino acids, eicosapentaenoic acid, indomethacin, ibuprofen, celecoxib, mirtazapine, olanzapine, melatonin, and clenbuterol.

27. The method of claim 26, wherein the combination of PDGFcc or a PDGFcc agonist and an additional therapeutic agent has a synergistic effect in the treatment or prevention of cachexia.

28. The method of any one of claims 20-27, wherein the cachexia is associated with cancer.

29. The method of claim 28, wherein the cancer is selected from cancers of the pancreas, oesophagus, stomach, lung, liver, or bowel.

30. A method to debulk a liposarcoma tumor and facilitate surgical removal of the liposarcoma tumor comprising administering an effective amount of a PDGFcc antagonist, an anti-PDGFcc antibody, or a fragment thereof, to the liposarcoma tumor prior to surgical removal.

31. The method of claim 30, comprising administering to the subject an effective amount of a combination therapy of: i) a PDGFcc antagonist, an anti-PDGFcc antibody, or an active fragment or a homolog thereof, and ii) at least one of a CCR2 antagonist or an anti-CCR2 antibody.


SEQUENCE LISTING (3 sequences)

SEQ ID NO: 1 - protein, 345 aa, Homo sapiens
MSLFGLLLLTSALAGQRQGTQAESNLSSKFQFSSNKEQNGVQDPQHERIITVSTNGSIHSPRFPHTYPRNTVLVWRLVAVEENVWIQLTFDERFGLEDPEDDICKYDFVEVEEPSDGTILGRWCGSGTVPGKQISKGNQIRIRFVSDEYFPSEPGFCIHYNIVMPQFTEAVSPSVLPPSALPLDLLNNAITAFSTLEDLIRYLEPERWQLDLEDLYRPTWQLLGKAFVFGRKSRVVDLNLLTEEVRLYSCTPRNFSVSIREELKRTDTIFWPGCLLVKRCGGNCACCLHNCNECQCVPSKVTKKYHEVLQLRPKTGVRGLHKSLTDVALEHHEECDCVCRGSTGG

SEQ ID NO: 2 - protein, 111 aa, Homo sapiens
VVDLNLLTEEVRLYSCTPRNFSVSIREELKRTDTIFWPGCLLVKRCGGNCACCLHNCNECQCVPSKVTKKYHEVLQLRPKTGVRGLHKSLTDVALEHHEECDCVCRGSTGG

SEQ ID NO: 3 - protein, 111 aa, Mus sp.
VVNLNLLKEEVKLYSCTPRNFSVSIREELKRTDTIFWPGCLLVKRCGGNCACCLHNCNECQCVPRKVTKKYHEVLQLRPKTGVKGLHKSLTDVALEHHEECDCVCRGNAGG